SEQUENCE LISTING (40 sequences)

SEQ ID NO: 1 - DNA, 33 bp, Artificial Sequence
  Synthetic oligonucleotide
tcgtcggcagcgtcagatgtgtataagagacag

SEQ ID NO: 2 - DNA, 34 bp, Artificial Sequence
  Synthetic oligonucleotide
gtctcgtgggctcggagatgtgtataagagacag

SEQ ID NO: 3 - DNA, 14 bp, Artificial Sequence
  Synthetic oligonucleotide
gacgctgccgacga

SEQ ID NO: 4 - DNA, 14 bp, Artificial Sequence
  Synthetic oligonucleotide
cgagcccacgagac

SEQ ID NO: 5 - DNA, 83 bp, Artificial Sequence
  Synthetic oligonucleotide
cgagcccacgagacccttggcacccgagaattccaagtatgctccttccgtccaaaaaaaaaaaaaaaaaaaaaaaaaaaaaa

SEQ ID NO: 6 - DNA, 83 bp, Artificial Sequence
  Synthetic oligonucleotide
cgagcccacgagacccttggcacccgagaattccagcgacgcagataaaccctaaaaaaaaaaaaaaaaaaaaaaaaaaaaaa

SEQ ID NO: 7 - DNA, 83 bp, Artificial Sequence
  Synthetic oligonucleotide
cgagcccacgagacccttggcacccgagaattccaccatctgaggtgtcagctaaaaaaaaaaaaaaaaaaaaaaaaaaaaaa

SEQ ID NO: 8 - DNA, 83 bp, Artificial Sequence
  Synthetic oligonucleotide
cgagcccacgagacccttggcacccgagaattccacgttgtactcagatctgtaaaaaaaaaaaaaaaaaaaaaaaaaaaaaa

SEQ ID NO: 9 - DNA, 83 bp, Artificial Sequence
  Synthetic oligonucleotide
cgagcccacgagacccttggcacccgagaattccagaccttgcgttattaactaaaaaaaaaaaaaaaaaaaaaaaaaaaaaa

SEQ ID NO: 10 - DNA, 82 bp, Artificial Sequence
  Synthetic oligonucleotide
cgagcccacgagacccttggcacccgagaattccaagttgtgtaagcggcccaaaaaaaaaaaaaaaaaaaaaaaaaaaaaa

SEQ ID NO: 11 - DNA, 83 bp, Artificial Sequence
  Synthetic oligonucleotide
cgagcccacgagacccttggcacccgagaattccaacttgcgtccctgcgagtaaaaaaaaaaaaaaaaaaaaaaaaaaaaaa

SEQ ID NO: 12 - DNA, 83 bp, Artificial Sequence
  Synthetic oligonucleotide
cgagcccacgagacccttggcacccgagaattccaacggagagtactaaatctaaaaaaaaaaaaaaaaaaaaaaaaaaaaaa

SEQ ID NO: 13 - DNA, 83 bp, Artificial Sequence
  Synthetic oligonucleotide
cgagcccacgagacccttggcacccgagaattccaacattgcgaccctttatcaaaaaaaaaaaaaaaaaaaaaaaaaaaaaa

SEQ ID NO: 14 - DNA, 83 bp, Artificial Sequence
  Synthetic oligonucleotide
cgagcccacgagacccttggcacccgagaattccagccttcgatgtacgatttaaaaaaaaaaaaaaaaaaaaaaaaaaaaaa

SEQ ID NO: 15 - DNA, 83 bp, Artificial Sequence
  Synthetic oligonucleotide
cgagcccacgagacccttggcacccgagaattccagctcgaggcaacgtaccgaaaaaaaaaaaaaaaaaaaaaaaaaaaaaa

SEQ ID NO: 16 - DNA, 83 bp, Artificial Sequence
  Synthetic oligonucleotide
cgagcccacgagacccttggcacccgagaattccaccgtcattgtgttaggacaaaaaaaaaaaaaaaaaaaaaaaaaaaaaa

SEQ ID NO: 17 - DNA, 83 bp, Artificial Sequence
  Synthetic oligonucleotide
cgagcccacgagacccttggcacccgagaattccacgtttcgggctcgaatctaaaaaaaaaaaaaaaaaaaaaaaaaaaaaa

SEQ ID NO: 18 - DNA, 83 bp, Artificial Sequence
  Synthetic oligonucleotide
cgagcccacgagacccttggcacccgagaattccacactagaataacggccgcaaaaaaaaaaaaaaaaaaaaaaaaaaaaaa

SEQ ID NO: 19 - DNA, 83 bp, Artificial Sequence
  Synthetic oligonucleotide
cgagcccacgagacccttggcacccgagaattccactatgctcacgttacgccaaaaaaaaaaaaaaaaaaaaaaaaaaaaaa

SEQ ID NO: 20 - DNA, 83 bp, Artificial Sequence
  Synthetic oligonucleotide
cgagcccacgagacccttggcacccgagaattccatacatctcccggtgcctgaaaaaaaaaaaaaaaaaaaaaaaaaaaaaa

SEQ ID NO: 21 - DNA, 83 bp, Artificial Sequence
  Synthetic oligonucleotide
cgagcccacgagacccttggcacccgagaattccagatgaacctagtcccgtgaaaaaaaaaaaaaaaaaaaaaaaaaaaaaa

SEQ ID NO: 22 - DNA, 83 bp, Artificial Sequence
  Synthetic oligonucleotide
cgagcccacgagacccttggcacccgagaattccatataaggtggtccgccagaaaaaaaaaaaaaaaaaaaaaaaaaaaaaa

SEQ ID NO: 23 - DNA, 83 bp, Artificial Sequence
  Synthetic oligonucleotide
cgagcccacgagacccttggcacccgagaattccacagctgagcttattatagaaaaaaaaaaaaaaaaaaaaaaaaaaaaaa

SEQ ID NO: 24 - DNA, 82 bp, Artificial Sequence
  Synthetic oligonucleotide
cgagcccacgagacccttggcacccgagaattccataacaatcctcttaagtaaaaaaaaaaaaaaaaaaaaaaaaaaaaaa

SEQ ID NO: 25 - DNA, 83 bp, Artificial Sequence
  Synthetic oligonucleotide
cgagcccacgagacccttggcacccgagaattccagaccgaggcgacgccaataaaaaaaaaaaaaaaaaaaaaaaaaaaaaa

SEQ ID NO: 26 - DNA, 83 bp, Artificial Sequence
  Synthetic oligonucleotide
cgagcccacgagacccttggcacccgagaattccactaggacgtcaagatgacaaaaaaaaaaaaaaaaaaaaaaaaaaaaaa

SEQ ID NO: 27 - DNA, 83 bp, Artificial Sequence
  Synthetic oligonucleotide
cgagcccacgagacccttggcacccgagaattccatgttccgatgggagaagcaaaaaaaaaaaaaaaaaaaaaaaaaaaaaa

SEQ ID NO: 28 - DNA, 83 bp, Artificial Sequence
  Synthetic oligonucleotide
cgagcccacgagacccttggcacccgagaattccaggttagccaaggagtattaaaaaaaaaaaaaaaaaaaaaaaaaaaaaa

SEQ ID NO: 29 - DNA, 83 bp, Artificial Sequence
  Synthetic oligonucleotide
cgagcccacgagacccttggcacccgagaattccattgccgatgcgcgtaaccaaaaaaaaaaaaaaaaaaaaaaaaaaaaaa

SEQ ID NO: 30 - DNA, 83 bp, Artificial Sequence
  Synthetic oligonucleotide
cgagcccacgagacccttggcacccgagaattccactcacgcctagaccactgaaaaaaaaaaaaaaaaaaaaaaaaaaaaaa

SEQ ID NO: 31 - DNA, 83 bp, Artificial Sequence
  Synthetic oligonucleotide
cgagcccacgagacccttggcacccgagaattccatttagctccgctcaacgcaaaaaaaaaaaaaaaaaaaaaaaaaaaaaa

SEQ ID NO: 32 - DNA, 83 bp, Artificial Sequence
  Synthetic oligonucleotide
cgagcccacgagacccttggcacccgagaattccactacatgtcgacgttggtaaaaaaaaaaaaaaaaaaaaaaaaaaaaaa

SEQ ID NO: 33 - DNA, 83 bp, Artificial Sequence
  Synthetic oligonucleotide
cgagcccacgagacccttggcacccgagaattccacggaggtatgctatatttaaaaaaaaaaaaaaaaaaaaaaaaaaaaaa

SEQ ID NO: 34 - DNA, 83 bp, Artificial Sequence
  Synthetic oligonucleotide
cgagcccacgagacccttggcacccgagaattccacgttctataaccactcgtaaaaaaaaaaaaaaaaaaaaaaaaaaaaaa

SEQ ID NO: 35 - DNA, 83 bp, Artificial Sequence
  Synthetic oligonucleotide
cgagcccacgagacccttggcacccgagaattccacacgattagggttcgtcgaaaaaaaaaaaaaaaaaaaaaaaaaaaaaa

SEQ ID NO: 36 - DNA, 83 bp, Artificial Sequence
  Synthetic oligonucleotide
cgagcccacgagacccttggcacccgagaattccatggagaactctcggtagcaaaaaaaaaaaaaaaaaaaaaaaaaaaaaa

SEQ ID NO: 37 - DNA, 83 bp, Artificial Sequence
  Synthetic oligonucleotide
cgagcccacgagacccttggcacccgagaattccattcaaccactgtgacagtaaaaaaaaaaaaaaaaaaaaaaaaaaaaaa

SEQ ID NO: 38 - DNA, 82 bp, Artificial Sequence
  Synthetic oligonucleotide
cgagcccacgagacccttggcacccgagaattccataccagttctagatgttaaaaaaaaaaaaaaaaaaaaaaaaaaaaaa

SEQ ID NO: 39 - DNA, 83 bp, Artificial Sequence
  Synthetic oligonucleotide
cgagcccacgagacccttggcacccgagaattccatcatacgggcgtaatgccaaaaaaaaaaaaaaaaaaaaaaaaaaaaaa

SEQ ID NO: 40 - DNA, 83 bp, Artificial Sequence
  Synthetic oligonucleotide
cgagcccacgagacccttggcacccgagaattccacttccaactctcgcatagaaaaaaaaaaaaaaaaaaaaaaaaaaaaaa

(19) United States
(12) Patent Application Publication
(10) Pub. No.: US 2022/0205035 A1
(43) Pub. Date: Jun. 30, 2022

(54) METHODS AND APPLICATIONS FOR CELL BARCODING

(71) Applicant: Board of Regents, The University of Texas System, Austin, TX (US)

(72) Inventors: Nicholas E. NAVIN, Houston, TX (US); Kaile WANG, Houston, TX (US)

(73) Assignee: Board of Regents, The University of Texas System, Austin, TX (US)

(21) Appl. No.: 17/601,405

(22) PCT Filed: Apr. 3, 2020
(86) PCT No.: PCT/US20/26619

§ 371 Date: Oct. 4, 2021

Related U.S. Application Data
(60) Provisional application No. 62/829,773, filed on Apr. 5, 2019.

(51) Int. Cl. C12Q 1/6869; G16B 30/00

(52) U.S. Cl. CPC C12Q 1/6869; G16B 30/00

(57) ABSTRACT

The current methods and compositions of the disclosure provide a platform for detecting the transcriptomic, genomic, or proteomic profile in relation to particular characteristics of a single cell, such as the location of a cell within a tissus. Accordingly, aspects of the disclosure relate to a method for barcoding eukaryotic cell nuclei comprising: transferring oligonucleotides into the nuclei of cells and performing single-cell analysis to identify the sequence of the barcode; wherein the oligonucleotides comprise a barcode region and a target region.

This application claims the benefit of U.S. Provisional Patent Application No. 62/829,773, filed Apr. 5, 2019, which is expressly incorporated by reference herein in its entirety.

### BACKGROUND

### 1. Field of the Invention

The invention relates to molecular biology techniques useful for diagnostics, research, and cellular assays.

### 2. Background

All living organisms are composed of individual cells that are spatially organized into tissues to form organ structures and perform biological functions. To understand how tissues work and are deregulated in diseases such as cancer, it is important to study their cell types composition and the spatial organization in tissues. Rapid progress in single cell genomics, transcriptomics, and epigenomics allow researchers to discover rare cell types, reconstruct cell lineages and study tumor microenvironment and tumor evolution. However, high-throughput single cell sequencing methods require the generation of cellular suspensions and thereby inherently lose all spatial information on the position of that cell in the original tissue section, which is critical for understanding tissue function and changes that occur during disease progression. Therefore, there is a need in the art for methods for spatially detection genomic, transcriptomic, or epigenomic information from cells.

### SUMMARY OF THE DISCLOSURE

The current methods and compositions of the disclosure provide a platform for detecting the transcriptomic, genomic, or proteomic profile in relation to particular characteristics of a single cell, such as the location of a cell within a tissus. Accordingly, aspects of the disclosure relate to a method for barcoding eukaryotic cell nuclei comprising: transferring a plurality of oligonucleotides into the nuclei of a plurality of cells and performing single-cell analysis to identify the sequence of the barcode; wherein each oligonucleotide comprises a barcode region and a target region.

Further aspects relate to a method for barcoding eukaryotic cell nuclei comprising: i) transferring oligonucleotides into the nuclei of cells; wherein the oligonucleotides comprise a barcode region and a target region; ii) combining the barcoded nuclei in a suspension and wherein the nuclear envelope of the barcoded nuclei is intact in the suspension; and iii) performing single-cell analysis of the suspension to identify the sequence of the barcode and the transcriptomic, proteomic, and/or genomic profile of the cell; wherein the barcode sequence is non-contiguous with endogenous DNA or RNA sequences and wherein the barcode corresponds to the endogenous location of a cell within a tissue section.

In some embodiments, the oligonucleotide is transferred into the nuclei of cells in a transposome complex. In some embodiments, the transposome complex facilitates the transfer of the oligonucleotide into the cell. In some embodiments, the oligonucleotide further comprises a transposome adaptor region that can be used to operatively link the oligonucleotide to a transposome complex. In some embodiments, the barcode corresponds to a cellular characteristic. In some embodiments, the characteristic comprises a location of the cell in a tissue, a cell type, a clonal population of cells, a patient sample, or a treatment condition. In specific embodiments, the cellular characteristic comprises the endogenous location of a cell within a tissue section. The barcode does not refer to a single known sequence put into one or more cells. The term “barcode” refers to a known sequence that identifies a unique cellular characteristic of the cell or a group of cells. Accordingly, the methods of the disclosure are useful for determining the unique cellular profile of at least or at most 2, 10, 25, 50, 100, 150, 200, 250, 300, 350, 400, 450, 500, 550, 600, 650, 700, 750, 800, 850, 900, 950, 1000, 1500, 2000, 2500, 3000, 3500, 4000, 4500, 5000, 5500, 6000, 6500, 7000, 7500, 8000, 8500, 9000, 9500, 10000, 15000, 20000, 25000, 30000, 35000, 40000, 45000, 50000, 75000, 100000, 125000, 150000, 175000, 200000, 300000, 400000, 500000, 600000, 700000, 800000, 1000000, 10⁷, 10⁸, 10⁹, 10¹⁰, 10¹¹, 10¹², 10¹³ or 10¹⁴ (or any derivable range therein) individual cells or group of cells that harbor the unique barcode marking the cell or group of cells to a unique cellular characteristic. The cellular profile may include a transcriptomic, genomic, or proteomic cellular profile. In some embodiments, the cellular profile includes specific protein analysis or interactions using assays described herein. In some embodiments, the cellular profile comprises expression of one or more RNAs, such as mRNA, miRNA, circRNA, etc., presence of one or more genomic sequences, such as disease-related genomic sequences, SNPs, variants, mutations, deletions, insertions, presence or absence of protein-protein interaction, and/or presence or absence of protein-nucleic interactions. Assays and methods described herein may be used to identify a cellular profile.

In some embodiments, the clonal population of cells comprises a clonal population of cancerous cells. The term “clonal population” refers to a population of cells derived from a single cell.

In some embodiments, the cells oligonucleotides are added to a suspension of cells to barcode many cells at the same time. In some embodiments, the oligonucleotides transferred to the cells have the same barcode. Thus, all the cells in the suspension are barcoded with the same barcode. In some embodiments, a second suspensions of cells is barcoded with a second barcode by adding oligonucleotides, all with the same second barcode. In some embodiments, one or more nth suspensions of cells are barcoded with an nth barcode, wherein n is 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, or 1000 (or any derivable range therein). In some embodiments, the barcoded suspensions of cells are mixed together prior to single cell analysis.

In some embodiments, the cells are within a tissue, and the cellular characteristic comprises the location of the cell within a tissue. In some embodiments, at least two cells at different locations in a tissue are each barcoded with a different barcode corresponding to the respective tissue locations of each of the cells. In some embodiments, at least 3, 4, 5, 6, 7, 8, 9, 10, 11, 12, 13, 14, 15, 16, 17, 18, 19, 20, 25, 30, 35, 40, 45, 50, 60, 70, 80, 90, 100, 125, 150, 175, 200, 225, 250, 275, 300, 325, 350, 375, 400, 425, 450, 475, 500, 525, 550, 575, 600, 625, 650, 675, 700, 725, 750, 775, 800, 825, 850, 875, 900, 950, 1000, 1100, 1200, 1300, 1400, 1500, 1600, 1700, 1800, 1900, 2000, 2200, 2400, 2600, 2800, 3000, 3200, 3400, 3600, 3800, 4000, 4200, 4400, 4600, 4800, 5000, 5500, 6000, 6500, 7000, 7500, 8000, 8500, 9000, 9500, 10000, 11000, 12000, 13000, 14000, 15000, 16000, 17000, 18000, 19000, 20000, 21000, 22000, 23000, 24000, 25000, 26000, 27000, 28000, 29000, 30000, 35000, 40000, 50000, 75000, 100000, 200000, 300000, 400000, 500000, 600000, 700000, 800000, 900000, or 1000000 (or any derivable range therein) cells at different locations in a tissue are each barcoded with a different barcode corresponding to the respective tissue locations of each of the cells.

In some embodiments, the cellular characteristic is a cell type, and wherein a first barcode corresponds to cells from a first cell type and a second barcode corresponds to cells from a second cell type. Embodiments of the disclosure relate to a first barcode corresponding to a first cellular characteristic, a second barcode corresponding to a second cellular characteristic, and an nth barcode corresponding to a nth cellular characteristic, wherein n is 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, or 1000 (or any derivable range therein). In some embodiments, multiple barcodes are provided to the cell and may correspond to multiple cellular characteristics. In some embodiments, the oligonucleotide comprises at least 2, 3, 4, 5, 6, 7, or 8 (or any derivable range therein) barcodes that each represent a different cellular characteristic for the particular cell.

In some embodiments, the cellular characteristic is a patient sample, and wherein a first barcode corresponds to cells from a first patient sample and a second barcode corresponds to cells from a second patient sample. In some embodiments, the cellular characteristic is a patient sample, and wherein a first barcode corresponds to cells from a first patient sample, a second barcode corresponds to cells from a second patient sample, and one or more nth barcodes corresponds to cells from one or more nth patient samples wherein n is 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, or 1000 (or any derivable range therein).

In some embodiments, the cellular characteristic is the location of the cell within a tissue, and wherein a first barcode corresponds to a first location and a second barcode corresponds to a second location. In some embodiments, the cellular characteristic is the location of the cell within a tissue, and wherein a first barcode corresponds to a first location, a second barcode corresponds to a second location, and one or more nth barcodes corresponds to one or more nth cellular locations wherein n is 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 200, 300, 400, 500, 600, 700, 800, 900, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900, 5000, 5100, 5200, 5300, 5400, 5500, 5600, 5700, 5800, 5900, 6000, 6100, 6200, 6300, 6400, 6500, 6600, 6700, 6800, 6900, 7000, 7100, 7200, 7300, 7400, 7500, 7600, 7700, 7800, 7900, 8000, 8100, 8200, 8300, 8400, 8500, 8600, 8700, 8800, 8900, 9000, 9100, 9200, 9300, 9400, 9500, 9600, 9700, 9800, 9900, 10000, 11000, 12000, 13000, 14000, 15000, 16000, 17000, 18000, 19000, 20000, 21000, 22000, 23000, 24000, 25000, 26000, 27000, 28000, 29000, 30000, 31000, 32000, 33000, 34000, 35000, 36000, 37000, 38000, 39000, 40000, 41000, 42000, 43000, 44000, 45000, 46000, 47000, 48000, 49000, 50000, 51000, 52000, 53000, 54000, 55000, 56000, 57000, 58000, 59000, 60000, 61000, 62000, 63000, 64000, 65000, 66000, 67000, 68000, 69000, 70000, 71000, 72000, 73000, 74000, 75000, 76000, 77000, 78000, 79000, 80000, 81000, 82000, 83000, 84000, 85000, 86000, 87000, 88000, 89000, 90000, 91000, 92000, 93000, 94000, 95000, 96000, 97000, 98000, 99000, 100000, 150000, 200000, 250000, 300000, 350000, 400000, 450000, 500000, 550000, 600000, 650000, 700000, 750000, 800000, 850000, 900000, 950000, 1000000, 1050000, or 1100000 (or any derivable range therein).

In some embodiments, the total area of barcoded cells within the tissue is greater than 1 mm². In some embodiments, the total area of barcoded cells within the tissue is greater than 1.5 mm². In some embodiments, the total area of barcoded cells within the tissue is greater than or at least 0.5, 0.6, 0.7, 0.8, 0.9, 1, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, or 3 mm² or any range derivable therein.

In some embodiments, the cellular characteristic is a treatment condition, and

wherein a first barcode corresponds to a first treatment condition and a second barcode corresponds to a second treatment condition. In some embodiments, the cellular characteristic is a treatment condition, and wherein a first barcode corresponds to a first treatment condition, a second barcode corresponds to a second treatment condition, and one or more nth barcodes corresponds to one or more nth treatment conditions wherein n is 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, or 1000 (or any derivable range therein).

In some embodiments, the method further comprises combining the barcoded nuclei in a suspension and wherein the nuclear envelope of the barcoded nuclei is intact in the suspension. In some embodiments, the method further comprises performing single-cell analysis of nucleic acids from the cellular nuclei. In some embodiments, the single-cell analysis comprises sequencing nucleic acids to determine the sequence of the barcode. In some embodiments, the single-cell analysis comprises sequencing cellular nucleic acids to determine the transcription or genomic profile of the single cell. In some embodiments, the single-cell analysis comprises determining the proteomic profile of the single cell. In some embodiments, the single-cell analysis comprises sequencing the nucleic acids. In some embodiments, the nucleic acids comprise RNA. In some embodiments, the single-analysis involves single-cell RNA sequencing to determine, quantitate, or identify one or more of RNA splicing, RNA-protein interaction, RNA modification, RNA structure or lincRNA, microRNA, mRNA, tRNA and circRNA analysis. In some embodiments, the analysis comprises one or more of drop-seq, InDrop, seq-well, fluidigm, BD biosciences, illumina bio-rad microdroplets, sci-seq microwell-seq, nanogrid-seq, 10× genomics RNA sequencing platform, SMART-seq, SMART-seq2, CEL-seq, CEL-seq2. In some embodiments, the nucleic acids comprise DNA. In some embodiments, the single-cell analysis comprises one or more of single cell DNA copy number profiling, single cell mutation detection, single cell structural variant detection, detection of DNA and protein interactions, DNA chromatin profiling, detection of DNA-DNA interactions, and detection of DNA epigenetic modifications. In some embodiments, the single cell analysis comprises one or more of single cell ChIP-seq, single cell 3C, single cell Hi-C, scDNase-seq, and scDanmID. In some embodiments, the single cell analysis comprises one or more of single cell Ribo-seq, single cell RIP-seq, and single cell CLIP-seq. In some embodiments, the single-cell analysis comprises one or more of 10× genomics CNV sequencing platform, mission bio, fluidigm, sci-seq, direct-tagmentation, sciATAC-seq, nano-well scATAC-seq, MDA, DOP-PCR, MALBAC, and LIANTI. In some embodiments, doublets are removed from single cell analysis.

In some embodiments, the single cell analysis includes an analysis that provides DNA and RNA sequence information from the same cell or epigenetics and RNA sequence information from the same cell. Examples of such methods include single cell DR-seq, G&T-seq, scMT-seq, scM&T-seq, scTrio-seq, scCOOL-seq, scNMT-seq, and SIDR-seq.

In some embodiments, the transcription or genomic profile comprises the profile of at least 1000 genes of the single cell. In some embodiments, the transcription or genomic profile comprises the profile of at least 500, 600, 700, 800, 900, 1000, 1100, 1200, 1300, 1400, 1500, 1600, 1700, 1800, 1900, 2000, 2100, 2200, 2300, 2400, 2500, 2600, 2700, 2800, 2900, 3000, 3250, 3500, 3750, 4000, 4250, 4500, 4750, 5000, 5500, 6000, 6500, 7000, 7500, 8000, 8500, 9000, 9500, 10000, 11000, 12000, 13000, 14000, 15000, 16000, 17000, 18000, 19000, 20000, 21000, 22000, 23000, 24000, 25000, 26000, 27000, 28000, 29000, 30000, 35000, 40000 or 50000 genes of the single cell (or any range derivable therein). In some embodiments, at least 2000 different barcodes are sequenced. In some embodiments, at least 300, 400, 500, 600, 700, 800, 900, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900, 5000, 5100, 5200, 5300, 5400, 5500, 5600, 5700, 5800, 5900, 6000, 6200, 6400, 6600, 6800, 7000, 7200, 7400, 7600, 7800, 8000, 8200, 8400, 8600, 8800, 9000, 9200, 9400, 9600, 9800, or 10000 (or any derivable range therein) different or total barcodes are sequenced.

In some embodiments, each cell contains, on average, one or two exogenously added barcodes. In some embodiments, the average number of barcodes per cell is one. In some embodiments, the average number of types of barcodes of the same sequence per cell is 1-2. In some embodiments, the average number of barcodes of the same sequence per cell is less than 2. In some embodiments, the average number of barcodes, such as barcodes of the same sequence, per cell is 0.8, 1, 1.2, 1.4, 1.6, 1.8, 2, 2.2, 2.4, 2.6, 2.8, 3, 3.5, or 4 (or any range derivable therein. Accordingly, the cell may contain multiple copies of the same barcode or of different barcode. In some embodiments, the cell comprises multiple copies of the same barcode. In some embodiments, each cell contains two distinct exogenously added barcodes (and/or multiple copies of each of the two distinct barcodes) and wherein the combination of the sequence of the two barcodes correspond to a cellular characteristic of each cell. In some embodiments, each cell comprises n distinct barcodes and wherein the combination of the sequences of the n barcodes corresponds to a cell characteristic of each cell and wherein n is an integer such as n=1, 2, 3, 4, 5, 6, 7, 8, 9, or 10. In some embodiments, the number of barcodes in a cell is the average number of barcodes in a cell that is in a population of cells. In some embodiments, the term barcode refers specifically to the barcode corresponding to a cellular characteristic. In some embodiments, each transposome complex comprises one or two oligonucleotides. In some embodiments, each transposome complex comprises at least, at most, or exactly 1, 2, 3, 4, 5, 6, 7, 8, 9, 10 or n oligonucleotides (or any derivable range therein), wherein n is an integer equal, at least, or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 (or any range derivable therein). In some embodiments, the transposome complex comprises at least two oligonucleotides. In some embodiments, the transposome complex comprises at least a first oligonucleotide comprising a first barcode and a second oligonucleotide comprising a second barcode and wherein the first and second barcode are different. In some embodiments, each transposome complex comprises at least, at most, or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 (or any range derivable therein) different oligonucleotides. In some embodiments, the number of oligonucleotides in a transposase complex is an average from a population of complexes.

In some embodiments, the nuclei is derived from or within a eukaryotic cell that is greater than 50 microns. In some embodiments, he nuclei is derived from or within a eukaryotic cell that is greater than 40, 45, 50, 55, 60, 65, 70, 75, 80, 85, 90, 95, 100, 125, 150, 175, or 200 microns (or any derivable range therein). In some embodiments, the nuclei is derived from or within a eukaryotic cell that comprises an irregular morphology. Irregular morphology may refer to a change in morphology of the cell due to oncogenic transformation or due to a disease state. In some embodiments, the nuclei is derived from or within a eukaryotic cell that has been previously frozen.

In some embodiments, the barcode sequence is non-contiguous with endogenous DNA or RNA sequences. The term non-contiguous, when referring to two nucleic acids means that the nucleic acids are not in the same nucleic acid molecule and are not covalently linked.

In some embodiments, the sequence comprising the barcode does not comprise endogenous nucleic acid sequences. In some embodiments, the method comprises sequencing of a barcode that is not integrated into cellular nucleic acids, such as genomic DNA or RNA that is endogenous to the cell. In some embodiments, the method excludes sequencing of a barcode that is integrated into genomic DNA or into endogenous RNA. In some embodiments, the sequence comprising the barcode does not comprise sequences from the cellular nucleic acids.

In some embodiments, the method excludes tagmentation of genomic nucleic acids by incorporation of the oligonucleotide of the transposome into genomic nucleic acids. In some embodiments, the barcode is not integrated into the genomic DNA or integrated into endogenous RNA. The term integrated implies that the barcode nucleic acids are in a covalent bond with the genomic DNA, such as with chromosomal DNA.

In some embodiments, the method further comprises isolating nucleic acids from the cells. In some embodiments, less than 1 ng nucleic acids is isolated from each cell. In some embodiments, less than 1000, 900, 800, 700, 600, 500, 400, 300, 200, 100, 75, 50, 25, 20, 15, 10, 5, 4, 3, 2, 1, 0.9, 0.8, 0.7, 0.6, 0.5, 0.4, 0.3, 0.2, 0.1, 0.08, 0.06, 0.04, 0.02, or 0.01 ng (or any derivable range therein is isolated from each cell.

In some embodiments, the transposome adaptor region comprises a transposase recognition sequence. In some embodiments, the transposome adaptor region comprises a complementary sequence capable of base-pairing with a transposome nucleic acid component. In some embodiments, the plurality of oligonucleotides comprises at least one oligonucleotide comprising a transposase recognition sequence and at least one oligonucleotide comprising a complementary sequence capable of base-pairing with a transposome nucleic acid component. In some embodiments, the method further comprises fragmentation of nucleic acids endogenous to the cell. In some embodiments, an adaptor region with one or more primer binding sites and/or barcodes is fused to one or both ends of the fragmented nucleic acids. In some embodiments, the fragmentation is performed prior to transferring the plurality of oligonucleotides into the plurality of cells. In some embodiments, the fragmentation is performed after transferring the plurality of oligonucleotides into the plurality of cells. In some embodiments, the fragmentation comprises tagmentation.

In some embodiments, the target region comprises one or more primer binding sites. In some embodiments, the target region comprises at least 1, 2, 3, or 4 primer binding sites. In some embodiments, the target region comprises a poly adenine region comprising at least 4 consecutive adenine nucleic acids. In some embodiments, the target region comprises a poly adenine region comprising at least 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 consecutive adenine nucleic acids (or any derivable range therein). In some embodiments, the target region comprises a universal primer binding region and a random primer binding region. In some embodiments, the target region and/or transposome adaptor region is unchanged relative to the cellular characteristic, but the barcode region is unique relative to the cellular characteristic.

In some embodiments, transferring the oligonucleotides into the cell comprises micropipetting oligonucleotides into or on top of each nucleus; printing oligonucleotides into or on top of each nucleus; releasing oligonucleotides from a substrate with cells deposited on top of the oligonucleotides and substrate; and acoustic liquid transfer of oligonucleotides to each nucleus.

In some embodiments, the oligonucleotide further comprises a cleavage site. In some embodiments, releasing oligonucleotides comprises restriction enzyme cleavage, nickase cleavage, UV photocleavage, or chemical cleavage of the oligonucleotide. In some embodiments, the substrate comprises a microarray. In some embodiments, the substrate comprises a bead, a polymer, or a microscope slide.

In some embodiments, the oligonucleotides are transferred to cell nuclei, and wherein the cells are in an endogenous location within a tissue section. In some embodiments, the cells are formalin fixed tissues. In some embodiments, the cells comprise paraffin embedded tissues. In some embodiments, the cells comprise frozen tissues. In some embodiments, the cells comprise tissues isolated from a mammal. In some embodiments, the cells comprise mammalian cells. In some embodiments, the cells comprise human, rat, mouse, cat, dog, horse, rabbit, pig, or goat cells.

In some embodiments, the transposome comprises Tn5, Sleeping Beauty, PiggyBac, Tn7 or MuA.

In some embodiments, the method comprises barcoding at least 100 cells, each with a different barcode corresponding to a different cell characteristic. In some embodiments, the method comprises barcoding at least 50, 100, 150, 200, 250, 300, 350, 400, 450, 500, 550, 600, 650, 700, 750, 800, 850, 900, 950, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4500, 5000, 5500, 6000, 6500, 7000, 7500, 8000, 8500, 9000, 9500, or 10000 cells (or any derivable range therein), each with a different barcode corresponding to a different cell characteristic or at least 80, 81, 82, 83, 84, 85, 86, 87, 88, 89, 90, 91, 92, 93, 94, 95, 96, 97, 98, or 99% (or any derivable range therein) of cells comprise a unique barcode.

In some embodiments, the transposome complexes are in a solution prior to transferring to the cellular nuclei; and wherein the solution comprises less than 0.05 μM oligonucleotide concentration. In some embodiments, the solution comprises 0.05-0.5 μM oligonucleotide. Such concentrations may be referred to as final concentrations in that they are the concentration of the oligo when it is in contact with the cell and/or cell nuclei. In some embodiments, the solution comprises 0.02-0.2 μM oligonucleotide. In some embodiments, the solution comprises 0.06-0.5 μM oligonucleotide. In some embodiments, the solution comprises less than, or comprises more than, or comprises about 0.005, 0.006, 0.007, 0.008, 0.009, 0.01, 0.015, 0.02, 0.025, 0.03, 0.035, 0.04, 0.045, 0.05, 0.055, 0.06, 0.065, 0.07, 0.075, 0.08, 0.085, 0.09, 0.1, 0.12, 0.14, 0.16, 0.18, 0.2, 0.22, 0.24, 0.26, 0.28, 0.3, 0.32, 0.34, 0.36, 0.38, 0.4, 0.42, 0.44, 0.46, 0.48, 0.5, 0.52, 0.54, 0.56, 0.58, 0.6, 0.62, 0.64, 0.66, 0.68, 0.7, 0.72, 0.74, 0.76, 0.78, 0.8, 0.85, 0.9, 0.95, or 1 (or any range derivable therein)μM oligonucleotide.

The terms “protein”, “polypeptide” and “peptide” are used interchangeably herein when referring to a gene product or functional protein.

The terms “contacted” and “exposed,” when applied to a cell, are used herein to describe the process by which an agent is delivered to a target cell or are placed in direct juxtaposition with the target cell or target molecule.

It is contemplated that the methods and compositions include exclusion of any of the embodiments described herein.

As used herein, the terms “or” and “and/or” are utilized to describe multiple components in combination or exclusive of one another. For example, “x, y, and/or z” can refer to “x” alone, “y” alone, “z” alone, “x, y, and z,” “(x and y) or z,” “x or (y and z),” or “x or y or z.” It is specifically contemplated that x, y, or z may be specifically excluded from an embodiment.

Throughout this application, the term “about” is used according to its plain and ordinary meaning in the area of cell biology to indicate that a value includes the standard deviation of error for the device or method being employed to determine the value.

The term “comprising,” which is synonymous with “including,” “containing,” or “characterized by,” is inclusive or open-ended and does not exclude additional, unrecited elements or method steps. The phrase “consisting of” excludes any element, step, or ingredient not specified. The phrase “consisting essentially of” limits the scope of described subject matter to the specified materials or steps and those that do not materially affect its basic and novel characteristics. It is contemplated that embodiments described in the context of the term “comprising” may also be implemented in to context of the term “consisting of” or “consisting essentially of.”

It is specifically contemplated that any limitation discussed with respect to one embodiment of the invention may apply to any other embodiment of the invention. Furthermore, any composition of the invention may be used in any method of the invention, and any method of the invention may be used to produce or to utilize any composition of the invention. Aspects of an embodiment set forth in the Examples are also embodiments that may be implemented in the context of embodiments discussed elsewhere in a different Example or elsewhere in the application, such as in the Summary of Invention, Detailed Description of the Embodiments, Claims, and description of Figure Legends.

Other objects, features and advantages of the present invention will become apparent from the following detailed description. It should be understood, however, that the detailed description and the specific examples, while indicating preferred embodiments, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1A-B. Overview of SNUBAR method with two different approaches for spatial barcoding of nuclei. Spatial barcoding of single nuclei by (A) microfluidic/micropipette depositing of spatial barcodes into tissue sections, or (B) using a custom microarray with spatial barcode oligonucleotide features pre-printed on the array that are delivered into the tissue sections.

FIG. 2A-B. Molecular Structure of spatial barcode oligonucleotide adaptors. (A) Spatial barcodes for single cell RNA sequencing, that contains a transposome binding sequence, spatial barcode sequence, and two platform-specific sequences (PCR handle, polyA tail). (B) Spatial barcodes for single cell DNA sequencing using direct tagmentation based chemistry, which contains transposome binding sequence and spatial barcode, and also a library-specific sequence for priming during PCR amplification.

FIG. 3A-B. Assembly of the Spatial Barcoded Transposome. (A) Hybridization of spatial barcode adapters to Transposome complex with universal adapters, showing an example application for single cell RNA-seq which includes a polyA priming tail. (B) Incorporation of spatial barcode adapters into the naked transposase to generate the spatial barcoded transposome.

FIG. 4A-D. Delivery System of the Spatial Transposome to Nuclei in Tissues. Several different approaches can be used to deliver the spatial barcode transposome or transposase to nuclei in the tissue sections as shown in this figure. (A) Sample barcoding of cells in suspension by adding the spatial transposome to different tubes. (B) Tissue barcoding by micropipetting spatial transposome complexes to different regions in tissue sections by hand, or using gaskets to concentrate the areas. (C) High-throughput automated micro-dispensing of transposome complexes to different spatial regions using acoustic liquid transfer systems, micromanipulators or microarray printers. (D) Using pre-printed custom microarrays with transposomes loaded to place tissue on the array and lyse the tissue to barcode different regions. Inset panel shows an example of using the pre-printed microarray transposome to deliver barcoded microarray probes into single cells/nuclei in more detail, in which each microarray feature, which contains universal sequence that complement to the sequence tail of transposome's adaptor, spatial barcodes, polyA (eg. for single cell RNA-seq) and linker sequence. The transposome with a universal adaptor assembles with the adapter features to form a barcoded transposome, then the barcoded transposome is released with the spatial barcode adapter, and enters the nuclei in tissue for barcoding.

FIG. 5—Library preparation and single cell transcriptomic profiling using spatial barcodes on the Drop-Seq platform. After the spatial transposome has delivered the spatial barcodes into the nucleus, the nuclei are used for Drop-seq WTA, in which the drop-seq beads hybridize to both the mRNA in the cell after lysis, as well as free spatial barcode adapters with platform-specific polyA adapters and PCR sequences. The droplets are subsequently released and the beads are used for reverse transcription and PCR amplification, after which libraries are generated for next-generation sequencing.

FIG. 6A-B—DNA size traces of the spatial barcode oligonucleotides and final cDNA libraries. This figure shows experimental data and quality control of the spatial barcode library size distributions (A) and the final cDNA sequencing library size traces from a pool of cancer cell lines pooled together (B) that were run on the tapestation (Agilent) system.

FIG. 7—Evaluation of the Efficiency of Spatial Barcode Delivery into Single Nuclei in Different Cell Lines. The number of spatial barcode counts identified in single cells from three cell lines after demultiplexing and analysis of the sequencing data.

FIG. 8—Spatial/sample Barcode Indexing and single cell RNA sequencing of 4 cell lines. High-dimensional analysis of single cell RNA and spatial barcodes for four cell lines that were pooled together for single cell RNA sequencing analysis.

FIG. 9—Percentage of different spatial barcodes for single cell RNA sequencing in four cell lines. Percentage of spatial barcodes delivered into single cells after 3′ high throughput single cell RNA sequencing of 4 different cell lines (SKN2, SK-BR-3, MDA-MB-231, MDA-MB-436).

FIG. 10—Spatial/sample Barcoding of 4 Cell Lines for Single Cell DNA Sequencing. Clustered heatmap of single cell copy number profiles from 4 different cell lines (SKN2, SK-BR-3, MDA-MB-231, MDA-MB-436) with spatial/sample barcoding after sequencing using Direct Tagmentation copy number profiling.

FIG. 11—Single nuclei Barcode counts of Four Cell Lines Using Single Cell DNA Sequencing. This figure shows that spatial/sample barcode percentages of four cell lines that were barcoded with different sequences and pooled together for direct tagmentation single cell copy number profiling and next-generation sequencing.

FIG. 12—Sample Barcoding of Three Cell Lines Without the Use of the Tn5 Delivery System. Normalized sample-specific barcode counts of single cells from three different cell lines (MDA-MB-231, SK-BR-3, MDA-MB-436) using high-concentration oligonucleotides without the Tn5 delivery system.

FIG. 13A-E. Overview of the SNuBar approach. (a) Fresh or frozen tissues is macro-dissected into small regions, after which single nuclei from each region are dissociated and incubated with unique barcoded transposomes (b) the loaded transposome delivers a spatial barcode into the nuclear suspensions from each tissue region, after which samples are pooled together into a single reaction. The barcode adapters delivered into intact nuclei serve as a synthetic target by providing a poly-T tail for priming and cell barcoding using microdroplet beads (c) High-throughput single nucleus RNA sequencing is performed using a microdroplet approach which generates a spatial barcode library and a cell barcode library for each nucleus. (d) computational matching of the spatial barcode library and cell barcode library of each nucleus, using the unique cell barcode identifier (e) mapping of single cell transcriptome data to the spatial tissue regions.

FIG. 14A-E Technical validation using cell line mixture experiments. (a) The upper panel shows gene counts detected per nucleus and the lower panel shows mitochondrial gene percentages in four different cell lines. (b) Percentage of barcodes in each cell is shown over the background levels across the four cell lines that were barcoded. (c) scatter plots of sample barcode counts in SK-BR-3 and MDA-MB-436 are shown to identify cross-contamination and doublets between the four different cell lines (d) Heatmap of normalized barcodes counts in the 4 different cell lines, indicating cells with single, multiple and no prevalent barcodes. (e) High-dimensional t-SNE plot of the expression data for the four cell lines, with singlets, multiplets and negative cells indicated.

FIG. 15A-F. The spatial organization of major cell types in a human breast tissue. (a) A human breast tissue was macro-dissected into 36 regions, and spatially barcoded with SNuBar, followed by pooling and snRNA-seq. (b) t-SNE plot of major cell types in the combined 36 spatial regions, in which 9 major cell type clusters were identified. (c) Normalized gene expression heatmap of top 10 differential markers for each cell type. (d) Pie charts of cell type frequencies and spatial locations in the 36 spatial regions, where the number on each pie chart represents the region ID, and the three major topographic areas of the breast tissue are labelled as A1-A3. (e) Hierarchical clustering of cell type proportions in each region and their spatial locations in the breast tissue. (f) Sankey plot mapping the 9 major breast cell types to three different spatial areas in the breast tissue.

FIG. 16A-G. The spatial co-localization of cell expression states in the human breast tissue. (a) t-SNE plot of cell types and expression states, showing clusters of fibroblasts, myeloid, epithelial and endothelial cells. (b) three fibroblast expression states (c) three myeloid expression states (d) three epithelial expression states, and (e) two endothelial expression states. (b-e) panels are organized from left to right showing high dimensional plots of the cell expression states for each cell type, clustered heatmaps of the top 10 genes per expression state, pie chart maps of expression state frequencies across the tissue regions, and Sankey plots mapping the expression states to the three major topographic areas. (f) Clustered heatmap of cell types and cell states frequencies across the spatial regions, showing three major clusters that correspond to different spatial areas. (g) Sankey plot mapping of cell types and expression states that co-localize to the three major topographic areas in the breast tissue.

FIG. 17A-M. Spatial organization of the tumor cells and microenvironment in an invasive breast cancer. (a) High-dimensional t-SNE plot of snRNA-seq data from a frozen estrogen-receptor positive breast tumor that was macro-dissected into 15 spatial regions. (b) Pie charts of cell type frequencies across 15 spatial regions in the breast tumor tissue (c) Sankey plot mapping of the major cell types to the macro-dissected spatial regions in the breast tumor tissue. (d) Clustered heatmaps of copy number aberrations calculated from the snRNA-seq read depth data, with consensus profiles of the three major clusters shown below. Black arrows in the consensus profiles show the major differences in genomic regions between clone 1 and clone 2. (e) high-dimensional expression plots of single cells from all spatial regions, with mapping of diploid and aneuploid copy number profiles inferred from the RNA read count data. (f) t-SNE plot of clustered expression data from the tumor cells. (g) mapping of the aneuploid and diploid cells to the tumor cell expression cluster data. (h) Pie charts of tumor subclone frequencies across the 15 spatial regions, indicating two major topographic areas (A1, A2) in the tumor tissue. (i) Sankey plot mapping the single cell data from the two tumor clones to the different spatial areas. (j) Differential expression of selected cancer genes enriched in either tumor clone 1 in the top panels, or enriched in tumor clone 2 in the bottom panels. Wilcoxon test indicates *: p<0.05, **: p<0.01, ***: p<0.001, ****: p<0.0001. (k) Top 10 significantly enriched GSEA signatures in T1 in the cancer hallmark pathway (adjusted FDR p<0.05). (1) Spatial distribution of the two macrophage expression programs across the 15 spatial regions and the two topographic areas. (m) Sankey plot showing the macrophage cell state colocalization to the two major topographic areas.

FIG. 18. The SNUBAR adapter consists of a complementary sequence to the transposome universal tail oligonucleotides, a PCR handle, a unique spatial/sample barcode and a synthetic polyA tail for priming on the high-throughput microdroplet snRNA-seq platform. The SNUBAR adapter is hybridized to the transposome complex with a universal tail. Separate transposomes are prepared with unique spatial/sample adapter barcodes (eg. 30-100) for each spatial region that will be barcoded. The loaded transposome is then incubate with the nuclear suspensions, after which the sample/spatial barcode will be delivered into the nuclear envelop and will either integrate into the genomic DNA or remain unintegrated in the nucleus.

FIG. 19. Counts of total transcripts in single nuclei in the 4 cell lines. SNUBAR barcoding of four different cell lines (SK-BR-3, MDA-MB-436, SKN-2, MDA-MB-231) from which transcript counts were quantified after single nucleus RNA sequencing.

FIG. 20A-B—High-dimensional plots of cell lines and doublet filtering. (a) t-SNE plot of four different sample barcoded cell lines (SK-BR-3, MDA-MB-436, SKN-2, MDAMB-231) that were used for SNUBAR barcoding and pooled together prior to single nucleus RNA sequencing on the 10× microdroplet platform. (b) cell line data after removal of cell multiplets identified as having multiple sample barcodes, in addition to negative cells with no prevalent barcodes.

FIG. 21A-D—Marker genes used to identify cell lines in mixture experiments. High dimensional t-SNE plots of the single nucleus RNA expression data from the combined four cell line data with SNUBAR barcodes. (a) Three markers of SKN-2 (COL1A1, COL1A2, POSTN), (b) of SK-BR-3 (ERBB2, KRT7, GRB7), (c) of MDA-MB-231 (CD74, KISS1, BIRC3) and (d) of MDA-MB-436 (PI3, CA9, SAA1) are shown in the feature plots.

FIG. 22—Percentage of sample barcode counts in cells relative to the background barcodes from the other cell lines Frequency of sample barcodes assigned to each cell line relative to contamination from other barcodes that entered the nucleus from unassigned cell lines.

FIG. 23. Scatter plots of cell multiplets and barcode cross-contamination. Scatter plots of sample barcode counts that were used to identify cross-contamination between the four different cell lines and cell multiplets.

FIG. 24—Number of nuclei detected in spatial regions from the matched normal breast tissue. Detected cell numbers in each of 36 macrodissected tissue regions from the human breast tissue after SNUBAR barcoding and single nucleus RNA sequencing.

FIG. 25A-C—Marker genes for epithelial cell types in normal breast tissue. Feature plots of known markers for three epithelial subtypes in the single nucleus RNA sequencing dataset from the human breast tissue. (a) Feature plots of KRT19, ESR1 and AR in the hormone responsive luminal cells, (b) KRT15 and LTF expression in the secretory luminal epithelial cells, and (c) Violin plots of ACTA2, SYNPO2, MYLK and KRT14 normalized gene expression for markers of the myoepithelial cells.

FIG. 26A-D—Marker genes for stromal cells in the normal breast tissue. Feature plots of established markers for three stromal cell types, including fibroblasts, adipocytes and endothelial cells. (a) Feature plots of marker gene expression of COL1A1, COL1A2, FN1 in fibroblast cells, and (b) ADIPOQ and PLIN1 expression in adipocytes. (c) Violin plots of gene expression for known markers PECAM1 and VWF in the vascular endothelial cells, and (d) expression of lymphatic endothelial cell markers MMRN1, PROX1 and PDPN) in the human breast tissue.

FIG. 27A-B—Marker genes for immune cells in normal breast tissue. Violin plots of known marker genes for immune cell types identified in the single nucleus RNA sequencing data from the normal breast tissue. (a) Violin plots of T-cell markers CD2, CD247, FYN and IL7R, and (b) general immune cell marker CD45 (PTPRC), and known macrophage markers MSR1 and MRC1 in the matched normal breast tissue.

FIG. 28—Clustered heatmap of fibroblast expression states and spatial regions in normal breast tissue. Clustering of the three fibroblast expression states (F1-F3) in 36 different spatial regions in the normal breast tissue. pct indicates the percentage of each fibroblast cell state in each spatial region.

FIG. 29A-C—Expression of proangiogenic and macrophage markers in the myeloid cells of the normal breast tissue. (a) Violin plot of single nucleus gene expression for the proangiogenic markers SPP1, NRP1, MMP9, HIF1A and CTSB, and the macrophage M2 markers MSR1, CD36, ITGAX (cd11c), ITGAM (cd11b), PPARG of the myeloid sub-cluster M2-1. (b) Violin plots of single nucleus gene expression for the M2 markers (MRC1, CD163, STAB1) in the macrophage subcluster M2-2. (c) Violin plots of established dendritic cell markers for AXL and TCF4, as well as the HLA genes (HLA-DRA, HLA-DRB1, HLA-DRB5, HLA-DPA1) in the myeloid cluster.

FIG. 30A-C—Clustered heatmaps of myeloid, epithelial and endothelial expression states and spatial regions in normal breast tissue. (a) Clustering of the three myeloid expression states M2-1, M2-2, DC, (b) clustering of the three epithelial expression states (LumHR+, LumHR−, MyoEpi), and (c) clustering of the two different endothelial expression states (LymEndo, VasEndo) in the 36 different spatial regions of the normal breast tissue. pct indicates the percentage of each fibroblast cell state in each region.

FIG. 31A-B—Feature plots of endothelial cell state markers. (a) Gene expression levels of lymphatic endothelial markers (CCL21, PROX1, PDPN, RELN) and (b) vascular endothelial markers (VWF, PECAM1, MCTP1, PALMD, MYRIP) are shown in two subpopulations of Endothelial cells.

FIG. 32A-B—Mitochondrial and ribosomal protein gene percentages in the frozen breast cancer sample. (a) Mitochondrial (MT) gene percentages detected in each single nucleus of the frozen breast tumor sample. (b) Ribosomal protein (RP) genes percentages detected in single nuclei from the frozen breast cancer samples.

FIG. 33—Clustered heatmap of top genes expressed in the 5 cell types from the frozen human breast tumor. Single nuclei RNA expression of the top 10 genes detected in each cluster corresponding to different cell types, including the tumor cells and 4 cell types in the microenvironment.

FIG. 34A-E—Known markers of cell types expressed in the single nuclei rna clusters from the human breast tumors. (a) Established fibroblast marker expression including COL1A1, FN1 and DCN, (b) general immune cell marker PTPRC (CD45), macrophage markers MSR1 and CD86, (c) luminal epithelial markers KRT18 and KRT19, (d) endothelial markers PECAM1 and VWF, and (e) T-cell markers CD3D and CD2.

FIG. 35—Expression of cancer-associated fibroblasts (CAFs) markers in the fibroblast population of the breast tumor. Violin plots of normalized gene expression for five CAFs markers (FAP, PDGFRB, COL1A1, POSTN, GREM1) across five cell type clusters identified by single nucleus RNA sequencing.

FIG. 36—Expression feature plots of CD8 cytotoxic T cell markers. Gene expression of CD8 cytotoxic T cell markers (GZMB, PRF1) in the clusters of cell types from the breast tumor sample.

FIG. 37—Immune and macrophages markers in the breast tumor. Violin plots show the single nucleus RNA expression level of immune cell gene (PTPRC, CD86) and M2 macrophages markers (MSR1, CD163, MRC1) in the breast tumor sample.

FIG. 38—Breast cancer genes expressed in the breast tumor tissue. Feature plots of 16 known breast cancer genes that are expression in the high-dimensional t-SNE plots of single nucleus RNA data from the breast tumor sample.

FIG. 39A-B—Spatial distribution of two tumor clones in 15 different regions. (a) Clustering of the two tumor clones (c1, c2) based on clonal frequencies, and (b) from the inferred copy number data. Pct indicates percentage of the clones in each spatial region.

FIG. 40A-B—Clustering of macrophage expression states in the breast tumor. (a) High-dimensional t-SNE plot of two macrophage subpopulations and (b) clustered heatmap of top 10 differential expression genes between the two macrophage subpopulations in the frozen human breast cancer tissues.

FIG. 41—Expression of gene markers for two macrophage subpopulations Violin plots of single nuclei RNA data showing the expression of gene markers for the two macrophage subpopulations in the breast tumor: (a) M2-2 markers and (b) M2-1 markers.

FIG. 42—Clustered heatmap of tumor clones and macrophage subpopulations in different spatial regions of the breast tumor Hierarchical clustering of the two tumor sub-populations (T1 and T2) and the two macrophage subpopulations (M2-1 and M2-2) defined by single nucleus RNA gene expression by spatial regions in the breast tumor.

FIG. 43A-B—High dimensional tSNE plot of the SNUBAR single cell RNA data using custom microarrays to deliver spatial barcodes into the tissue of a DCIS patient (A) and normalized gene expression heatmap of top 10 differential markers for each cell type (B).

FIG. 44A-C—Spatial distribution of single cells detected using the custom microarray based SNUBAR method. (A) Spatial distributions in X-Y coordinates in the DCIS tissue sections based on the SNUBAR spatial barcodes. (B) Bright field of tissue under macroscope before dissociation. (C) DAPI staining of nuclei in the DCIS tissue section before dissociation.

FIG. 45A-E—This figure shows using single, double or multiple barcode oligos to prepare barcoded transposomes for multiplexing. (A) Barcodes with same barcode sequences are assembled with transposome containing two universal tails, in this example we only show barcodes with the same universal tails, however another possibility is to use a single barcodes sequence with two or multiple universal tails to hybridize with the transposome universal tails. (B) Barcodes with two different barcode sequences are assembled with two different universal tails in the transposome. Barcodes with same barcode sequences could have different universal tails that hybridize with the transposome universal tails. (C) Barcodes with two different barcodes sequences, but with same universal tails are assembled together with the transposome. (D) Barcodes with multiple different barcodes sequences, but with same universal tails are assembled with transposome. (E) Barcodes with multiple different barcodes sequences, but with two different universal tails are assembled with the transposome. All of the above scenarios in A-E shown demonstrate how to barcode single cell/nucleus using single or combinatorial barcodes assembled with the transposase or transposome, alternatively one can assemble the barcoded transposomes separately, then mix them together to obtain a mixed barcoded transposome.

### DESCRIPTION OF ILLUSTRATIVE EMBODIMENTS

The inventors have created a system, termed spatial nucleus barcoding (SNUBAR), that enables spatially barcoding of single nuclei in tissue sections before dissociating the tissue into nuclear suspensions for high-throughput sequencing. SNUBAR involves four steps: 1) assembling a spatial barcode transposome, 2) applying the spatial transposome across different regions in tissue sections, 3) dissociating the tissue into a nuclear suspension for high-throughput single cell sequencing, and 4) mapping the spatial barcode indexes to the single cell genomics data to determine the original (X, Y) position of the cell in the tissue section. In some embodiments, step (1) and (2) can occur together. In some embodiments, the tissue may be dissociated first, and then step (1) and/or (2) may be performed either together or sequentially. This approach can be applied broadly to fresh and frozen tissues and is compatible with a variety of downstream single cell sequencing approaches, such as microfluidic-based high throughput single cell RNA sequencing methods such as Drop seq, InDrop, Seq-Well, Microwell-seq, Nanogrid seq, 10× genomics RNA sequencing platform, or low-throughput methods such as SMART-seq, SMART-seq2, CEL-seq, CEL-seq2. In addition to single cell RNA sequencing methods, this approach can be used for single cell DNA analysis such as 10× genomics CNV sequencing platform, sci-seq, direct-tagmentation or epigenomic sequencing analysis such as sciATAC-seq and Nano-well scATAC-seq. In summary, SNUBAR can link spatial information from histopathology or imaging of tissue sections to single cell genomic data, and is likely to have broad applications in studying premalignant cancers, invasive cancers, disease tissues that are defined by histopathology. The approach can also be used in many research applications to study the basic biology of immunology, development, cancer progression or neurobiology.

### I. Oligonucleotides

Embodiments of the disclosure relate to oligonucleotides comprising a barcode region, a target region, and transposome adaptor region, which are further described below. The terms “oligonucleotide:” “polynucleotide,” and “nucleic acid” may be used interchangeable and include linear oligomers of natural or modified monomers or linkages, including deoxyribonucleosides, ribonucleosides, α-anomeric forms thereof, peptide nucleic acids (PNAs), and the like, capable of specifically binding to a target polynucleotide by way of a regular pattern of monomer-to-monomer interactions, such as Watson-Crick type of base pairing, base stacking, Hoogsteen or reverse Hoogsteen types of base pairing, or the like. Usually monomers are linked by phosphodiester bonds or analogs thereof to form oligonucleotides ranging in size from a few monomeric units, e.g. 3-4, to several tens of monomeric units. Whenever an oligonucleotide is represented by a sequence of letters, such as “ATGCCTG,” it will be understood that the nucleotides are in 5′→3′ order from left to right and that “A” denotes deoxyadenosine, “C” denotes deoxycytidine, “G” denotes deoxyguanosine, and “T” denotes thymidine, unless otherwise noted. Analogs of phosphodiester linkages include phosphorothioate, phosphorodithioate, phosphoranilidate, phosphoramidate, and the like. It is clear to those skilled in the art when oligonucleotides having natural or non-natural nucleotides may be employed, e.g. where processing by enzymes is called for, usually oligonucleotides consisting of natural nucleotides are required.

The nucleic acid may be an “unmodified oligonucleotide” or “unmodified nucleic acid,” which refers generally to an oligomer or polymer of ribonucleic acid (RNA) or deoxyribonucleic acid (DNA). In some embodiments a nucleic acid molecule is an unmodified oligonucleotide. This term includes oligonucleotides composed of naturally occurring nucleobases, sugars and covalent internucleoside linkages. The term “oligonucleotide analog” refers to oligonucleotides that have one or more non-naturally occurring portions which function in a similar manner to oligonucleotides. Such non-naturally occurring oligonucleotides are often selected over naturally occurring forms because of desirable properties such as, for example, enhanced cellular uptake, enhanced affinity for other oligonucleotides or nucleic acid targets and increased stability in the presence of nucleases. The term “oligonucleotide” can be used to refer to unmodified oligonucleotides or oligonucleotide analogs.

Specific examples of nucleic acid molecules include nucleic acid molecules containing modified, i.e., non-naturally occurring internucleoside linkages. Such non-naturally internucleoside linkages are often selected over naturally occurring forms because of desirable properties such as, for example, enhanced cellular uptake, enhanced affinity for other oligonucleotides or nucleic acid targets and increased stability in the presence of nucleases. In a specific embodiment, the modification comprises a methyl group.

Nucleic acid molecules can have one or more modified internucleoside linkages. As defined in this specification, oligonucleotides having modified internucleoside linkages include internucleoside linkages that retain a phosphorus atom and internucleoside linkages that do not have a phosphorus atom. For the purposes of this specification, and as sometimes referenced in the art, modified oligonucleotides that do not have a phosphorus atom in their internucleoside backbone can also be considered to be oligonucleosides.

Modifications to nucleic acid molecules can include modifications wherein one or both terminal nucleotides is modified. One suitable phosphorus-containing modified internucleoside linkage is the phosphorothioate internucleoside linkage. A number of other modified oligonucleotide backbones (internucleoside linkages) are known in the art and may be useful in the context of this embodiment. Representative U.S. patents that teach the preparation of phosphorus-containing internucleoside linkages include, but are not limited to, U.S. Pat. Nos. 3,687,808; 4,469,863; 4,476,301; 5,023,243, 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; 5,194,599; 5,565,555; 5,527,899; 5,721,218; 5,672,697 5,625,050, 5,489,677, and 5,602,240 each of which is herein incorporated by reference.

Modified oligonucleoside backbones (internucleoside linkages) that do not include a phosphorus atom therein have internucleoside linkages that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having amide backbones; and others, including those having mixed N, O, S and CH2 component parts.

Representative U.S. patents that teach the preparation of the above non-phosphorous-containing oligonucleosides include, but are not limited to, U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; 5,792,608; 5,646,269 and 5,677,439, each of which is herein incorporated by reference.

Oligomeric compounds can also include oligonucleotide mimetics. The term mimetic as it is applied to oligonucleotides is intended to include oligomeric compounds wherein only the furanose ring or both the furanose ring and the internucleotide linkage are replaced with novel groups, replacement of only the furanose ring with for example a morpholino ring, is also referred to in the art as being a sugar surrogate. The heterocyclic base moiety or a modified heterocyclic base moiety is maintained for hybridization with an appropriate target nucleic acid.

Oligonucleotide mimetics can include oligomeric compounds such as peptide nucleic acids (PNA) and cyclohexenyl nucleic acids (known as CeNA, see Wang et al., J. Am. Chem. Soc., 2000, 122, 8595-8602). Representative U.S. patents that teach the preparation of oligonucleotide mimetics include, but are not limited to, U.S. Pat. Nos. 5,539,082; 5,714,331; and 5,719,262, each of which is herein incorporated by reference. Another class of oligonucleotide mimetic is referred to as phosphonomonoester nucleic acid and incorporates a phosphorus group in the backbone. This class of olignucleotide mimetic is reported to have useful physical and biological and pharmacological properties in the areas of inhibiting gene expression (antisense oligonucleotides, ribozymes, sense oligonucleotides and triplex-forming oligonucleotides), as probes for the detection of nucleic acids and as auxiliaries for use in molecular biology. Another oligonucleotide mimetic has been reported wherein the furanosyl ring has been replaced by a cyclobutyl moiety.

Nucleic acid molecules can also contain one or more modified or substituted sugar moieties. The base moieties are maintained for hybridization with an appropriate nucleic acid target compound. Sugar modifications can impart nuclease stability, binding affinity or some other beneficial biological property to the oligomeric compounds. Representative modified sugars include carbocyclic or acyclic sugars, sugars having substituent groups at one or more of their 2′, 3′ or 4′ positions, sugars having substituents in place of one or more hydrogen atoms of the sugar, and sugars having a linkage between any two other atoms in the sugar. A large number of sugar modifications are known in the art, sugars modified at the 2′ position and those which have a bridge between any 2 atoms of the sugar (such that the sugar is bicyclic) are particularly useful in this embodiment. Examples of sugar modifications useful in this embodiment include, but are not limited to compounds comprising a sugar substituent group selected from: OH; F; O-, S-, or N-alkyl; or O-alkyl-O-alkyl, wherein the alkyl, alkenyl and alkynyl may be substituted or unsubstituted C1 to C10 alkyl or C2 to C10 alkenyl and alkynyl. Particularly suitable are: 2-methoxyethoxy (also known as 2′-O-methoxyethyl, 2′-MOE, or 2′-OCH2CH2OCH3), 2′-O-methyl (2′-O—CH3), 2′-fluoro (2′-F), or bicyclic sugar modified nucleosides having a bridging group connecting the 4′ carbon atom to the 2′ carbon atom wherein example bridge groups include —CH2-O—, —(CH2)2-O— or —CH2-N(R3)-O wherein R3 is H or C1-C12 alkyl.

Nucleic acid molecules can also contain one or more nucleobase (often referred to in the art simply as “base”) modifications or substitutions which are structurally distinguishable from, yet functionally interchangeable with, naturally occurring or synthetic unmodified nucleobases. Such nucleobase modifications can impart nuclease stability, binding affinity or some other beneficial biological property to the oligomeric compounds. As used herein, “unmodified” or “natural” nucleobases include the purine bases adenine (A) and guanine (G), and the pyrimidine bases thymine (T), cytosine (C) and uracil (U). Modified nucleobases also referred to herein as heterocyclic base moieties include other synthetic and natural nucleobases, many examples of which such as 5-methylcytosine (5-me-C), 5-hydroxymethyl cytosine, 7-deazaguanine and 7-deazaadenine among others.

Heterocyclic base moieties can also include those in which the purine or pyrimidine base is replaced with other heterocycles, for example 7-deaza-adenine, 7-deazaguanosine, 2-aminopyridine and 2-pyridone. Some nucleobases include those disclosed in U.S. Pat. No. 3,687,808, those disclosed in The Concise Encyclopedia Of Polymer Science And Engineering, pages 858-859, Kroschwitz, J. I., ed. John Wiley & Sons, 1990, those disclosed by Englisch et al., Angewandte Chemie, International Edition, 1991, 30, 613, and those disclosed by Sanghvi, Y. S., Chapter 15, Antisense Research and Applications, pages 289-302, Crooke, S. T. and Lebleu, B., ed., CRC Press, 1993. Certain of these nucleobases are particularly useful for increasing the binding affinity of the oligomeric compounds. These include 5-substituted pyrimidines, 6-azapyrimidines and N-2, N-6 and O-6 substituted purines, including 2 aminopropyladenine, 5-propynyluracil and 5-propynylcytosine.

The oligonucleotide oligos may be at least 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, or 400 nucleotides in length (or any derivable range therein).

### B. Barcode

The oligonucleotides of the disclosure comprise a barcode region, which can be used to identify a cellular characteristic. The barcode region can be a polynucleotide of at least, at most, about, or exactly 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 150, 200 or more (or any range derivable therein) nucleotides in length. The barcode may comprise one or more universal PCR regions, adaptors (such as adaptors for making cDNA libraries), linkers, or a combination thereof. The barcode region may also include a molecular index region (MI) which can be used to count how many barcode sequences are delivered into each cell or nucleus. The MI may be 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 150, 200 or more (or any range derivable therein) nucleotides in length.

The cellular characteristics that can be identified by the barcode region include cell type; tissue type; treatment condition; such as treatment with a compound, a nucleic acid, a polypeptide, or an antibody; location of a cell within a tissue; or patient identity. In certain embodiments, the cellular characteristic comprises the location of cell within a tissue. In certain embodiments, the cellular characteristic comprises the planar location of a cell within a tissue. The barcode may be specific for a cell or a population of cells, such that isolation of sequencing of the barcode after combining multiple differentially barcoded cells or populations of cells identifies the cellular characteristic of the cell or population of cells. The cellular characteristic can then be associated with other sequencing data or analysis of the cell or population of cells. For example, the analysis may include epigenomic, genomic, or transcriptomic information obtained by single-cell analysis of mRNA or DNA.

In some embodiments, the barcode is unique to one cell. In some embodiments, the barcode is unique to a population of cells, such as about 2, 3, 4, 5, 6, 7, 8, 9, 10, 50, 100, 500, 1000, 5000, 10000, 25000, 50000, 100000, 500000, or 1000000 (or any derivable range therein) cells. In some embodiments, the oligonucleotide comprising the barcode is printed on a substrate. In some embodiments, cells are deposited on top of the substrate with the printed barcode. In this instance, the barcode may represent an X and Y coordinate of the substrate, which then corresponds to a location of a cell or cells deposited on the substrate. The cells may be deposited as a tissue section. For example, sectioning may be done on a tissue. For example, a steel or diamond knife mounted in a microtome or ultramicrotome can be used to cut tissue sections of defined thickness, such as 20, 30, 40, 50, 100, 200, 500 or 1000 nanometers or 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 35, 36, 37, 38, 39, 40, 41, 42, 43, 44, 45, 46, 47, 48, 49, or 50 micrometers, which can then be mounted to a substrate, such as a microscope slide. In some embodiments, the microscope slide has pre-printed oligonucleotides of the disclosure.

Sections can be cut through the tissue in a number of directions. For pathological evaluation of tissues, vertical sectioning, (cut perpendicular to the surface of the tissue to produce a cross section) is the usual method. Horizontal (also known as transverse or longitudinal) sectioning, cut along the long axis of the tissue, is often used in the evaluation of the hair follicles and pilosebaceous units. Tangential to horizontal sectioning is used in Mohs surgery and in methods of CCPDMA.

The tissue may be fixed or unfixed. In some embodiments, the tissue is fixed prior to deposition onto a substrate. In some embodiments, the tissue comprises a formalin fixed section. In some embodiments, the section comprises a cryosection. In some embodiments, the tissue may undergo certain treatments to allow the uptake of materials, such as oligonucleotides deposited on a substrate. For example, the tissue may undergo permeabilization to allow for uptake of oligonucleotide from a transfer method described herein.

In some embodiments, the tissue is stained with one or more laboratory stains such as haemoatoxylin, eosin, toluidine blude, Masson's trichrome stain, Mallory's trichrome stain, Weigert's elastic stain, Heidenhain's AZAN trichrome stain, silver stain, Whright's stain, Orcein stain, DAPI, Hoechst stains, SYTO stains, propidium iodide, TO-PRO-3, SYTOX stains and Periodic acid-schiff stain. Alternative histological techniquest may be used, such as plastic embedding.

In some embodiments, the tissue has be subjected to an analysis either before or after transfer of the oligonucleotide. The analysis may include fluorescent in situ hybridization or immunohistochemistry. In some embodiments, the cellular characteristic may be a cell that provides a positive fluorescent signal in an analysis technique.

The barcodes are quantified or determined by methods known in the art, including quantitative sequencing (e.g., using an Illumina® sequencer) or quantitative hybridization techniques (e.g., microarray hybridization technology or using a Luminex® bead system). Sequencing methods are further described herein.

### C. Target Region

The target region may be nucleic acids that aid in the detection, amplification, sequencing, and or library preparation of the oligonucleotide and/or other nucleic acids in the barcoded cell. In some embodiments, the target region may be used as a primer binding site for amplification of DNA or RNA. The target region may be specific to an analysis technique applied to the single cells. The analytic techniques may further comprise another barcode that is specific for the nucleic acids in the cell, such as the cellular DNA or RNA. In some embodiments, a cellular barcode, such as one that identifies the cellular nucleic acids, may be amplified with or on the same nucleic acid as a barcode from an oligonucleotide of the disclosure, such as a barcode that identifies a cellular characteristic. These single-cell analysis techniques are further described below. The single-cell analysis techniques described herein may be used in embodiments of the disclosure. For example, the library specific sequence may comprise a primer binding sequence and a polyA region. The poly A region can bind to polyT oligonucleotides in RNA analysis methods. The primer binding sequence can be used as PCR primer binding sequence to amplify and sequence spatial barcode sequence and/or cellular barcode sequences. As another example, if the barcoded nuclei will be sequenced by high-throughput single cell DNA sequencing for copy number (eg. direct tagmentation based chemistry), the target specific sequences can be universal sequences, where the universal sequence will be used to mark spatial barcode positions. The target sequence can be customized based on different downstream sequencing library construction methods and applications.

### D. Transposome Adaptor Region

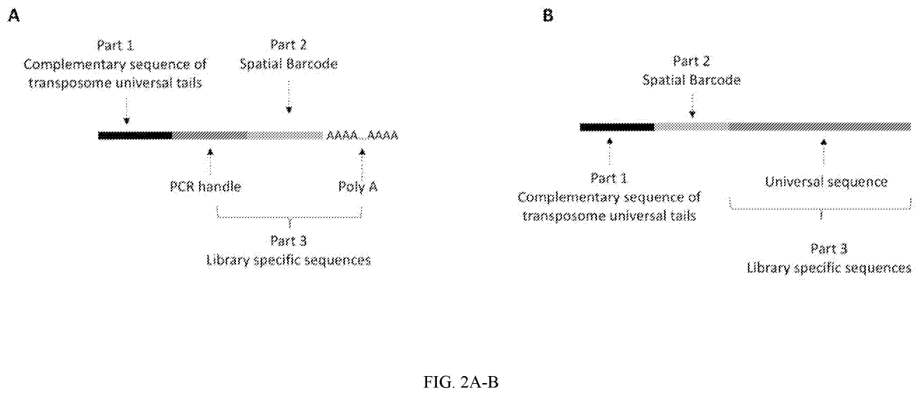

The transposome adaptor region provides for a sequence that links/binds the oligonucleotide to the transposase or transposome complex. For example, the transposome adaptor region may comprise a sequence that binds directly to the transposase enzyme, or a sequence that binds to complementary universal oligonucleotide adapters in the transposome. This is further illustrated in FIG. 2 of Example 1. Examples include adaptors such as TCGTCGGCAGCGTCagatgtgtataagagacag (SEQ ID NO:1) and GTCTCGTGGGCTCGGagatgtgtataagagacag (SEQ ID NO:2), (Capital letter: universal sequence, lowercase letter: mosaic sequence that will be recognized and bound by Tn5 Transposase) for use in systems that have a Tn5 transposome. In certain embodiments, the transposome adaptor region of barcode oligonucleotide could be designed to be complementary to the universal adaptor of SEQ ID NO:1 or 2. Structures of exemplary oligonucleiotides comprising a transposome adaptor region include the following: Such as the following barcode oligonucleotides: (1) 5′-GACGCTGCCGACGA (SEQ ID NO:3)---PCR handle sequence---spatial/sample barcode sequence-poly A-3′ (SEQ ID NO:3 is compliment of SEQ ID NO:1 universal sequence) and (2) 5′-CGAGCCCACGAGAC (SEQ ID NO:4)---PCR handle sequence---spatial/sample barcode sequence-poly A-3′ (SEQ ID NO:4 is a compliment of SEQ ID NO:2 universal sequence).

### II. Transposome Complexes

### A. Transposase

The transposase may be any transposase that binds to an oligo to form a transposome complex. In some embodiments, the transposase is a DDE transposase. These transposases carry a triad of conserved amino acids: aspartate (D), aspartate (D) and glutamate (E), which are required for the coordination of a metal ion required for catalysis, although the DDE chemistry can be integrated into the transposition cycle in differing ways. These employ a cut-and-paste mechanism of the original transposon. This family includes the maize Ac transposon, as well as the Drosophila P element, bacteriophage Mu, Tn5 and Tn10, Mariner, IS10, and IS50.

In some embodiments, the transposase is a Tyrosine (Y) transposase. These also use a cut-and-paste mechanism of transposition, but employ a site-specific tyrosine residue. The transposon is excised from its original site (which is repaired); the transposon then forms a closed circle of DNA, which is integrated into a new site by a reversal of the original excision step. These transposons are usually found only in bacteria, and include Kangaroo, Tn916, and DIRS1.

In some embodiments, the transposase is a Serine (S) transposases. These transposases use a cut-and-paste (cut-out/paste-in) mechanism of transposition involving a circular DNA intermediate, which is similar to that of tyrosine transposases, only they employ a site-specific serine residue. These transposons are usually found only in bacteria, and include Tn5397 and IS607.

In some embodiments, the transposase is a Rolling-circle (RC), or Y2 transposase. These employ either a copy-in mechanism, where they copy a single strand directly into the target site by DNA replication, so that the old (template) and new (copied) transposons both have one newly synthesized strand. These transposons usually employ host DNA replication enzymes. Examples include IS91 and helitrons.

In some embodiments, the transposase is a reverse transposase. In some embodiments, the oligonucleotide comprises class 2 transposon elements.

Examples of transposases are provided in the following table:

| UniProt | Protein name | Organism |
|---|---|---|
| TRA1_MAIZE | Putative AC transposase | Zea mays |
| (P08770) | | Maize |
| HOBOT_DROME | Transposable element | Drosophila melanogaster |
| (P12258) | Hobo transposase | Fruit fly |
| Q38743_ANTMA | Tam3-transposase | Antirrhinum majus |
| (Q38743) | | Garden snapdragon |
| TRA_BPMU | Transposase | Bacteriophage Mu |
| (P07636) | | Virus |
| PELET_DROME | Transposable element P | Drosophila melanogaster |
| (Q7M3K2) | transposase | Fruit fly |
| Q3QBD4_9GAMM | Transposase Tn5 | Shewanella baltica |
| (Q3QBD4) | | Bacteria |
| Q46731_ECOLI | Transposase | Escherichia coli |
| (Q46731) | | Bacteria |
| TC1A_CAEEL | Transposable element | Caenorhabditis elegans |
| (P03934) | Tc1 transposase | Nematode worm |
| Q583L2_9TRYP | Transposase of Tn10 | Trypanosoma brucei |
| (Q583L2) | | Trypanosome |

In some embodiments, the methods of the disclosure utilize a transposome with universal adaptors. Such complexes are commercially available. For Example, Tn5 transposome is available from Illumina, TDE1 transposome is available from the Nextera DNA Library Prep Kit, ATM transposome is available from the Nextera XT DNA Library Prep Kit.

### B. Transfer of Complexes into Cells

Embodiments of the disclosure relate to the transfer of transposome complexes into cells, which then can enter nuclei to provide a barcoded cellular nuclei. In some embodiments, the transposome complexes are transferred into cells by manual pipetting of the complexes on top of the cells. Manual pipetting, such as micro-pipetting, may be performed with the aid of a microscope. A composition comprising transposon complexes may be pipetted on top of each cell to allow for the transfer of the complex into the cell. In some embodiments, the transposome complex is deposited on top of the nuclei. In some embodiments, a microfluidic depositing system is used. In some embodiments, a microarray printer or liquid transfer system is used to transfer the transposome complexes to the cells or nuclei. In some embodiments, a microarray is utilized. The oligonucleotide or a pre-assembled transposome may be printed on the surface of a microarray. In some embodiments, the oligonucleotide is loaded onto a substrate, such as a microarray, and transposome complexes comprising an oligonucleotide that binds, through base complementarity, to the transposome adaptor region of the oligonucleotide on the surface of the microarray is added to form an attachment of the oligonucleotides on the surface of the substrate to the transposon complexes. After loading the transposome on the microarray, tissue sections can be applied to the substrate, for example applied on top of the barcoded transposome substrate. In some embodiments, the method further comprises permeabilizing the tissue. In some embodiments, the methods comprise or further comprise releasing the barcodes from the substrate. In some embodiments, the oligonucleotide comprises a cleavage site, such as a restriction enzyme site. In some embodiments, releasing oligonucleotides comprises restriction enzyme cleavage, nickase cleavage, UV photocleavage, or chemical cleavage of the oligonucleotide.

A nucleic acid array can comprise at least 1, 2, 3, 4, 5, 6, 7, 8, 9, 10, 15, 20, 25, 30, 35, 40, 45, 50, 60, 70, 80, 90, 100, 150, 200, 250 or more different polynucleotide oligos, which may hybridize to different and/or the same biomarkers, transposome universal adaptors, oligonucleotides. The probe density on the array can be in any range. In some embodiments, the density may be 50, 100, 200, 300, 400, 500 or more oligos/cm².

Specifically contemplated are chip-based nucleic acid technologies such as those described by Hacia et al. (1996) and Shoemaker et al. (1996). Briefly, these techniques involve quantitative methods for analyzing large numbers of genes rapidly and accurately. By tagging genes with oligonucleotides or using fixed probe arrays, one can employ chip technology to segregate target molecules as high density arrays and screen these molecules on the basis of hybridization (see also, Pease et al., 1994; and Fodor et al, 1991). It is contemplated that this technology may be used in conjunction with the methods described herein.

Certain embodiments may involve the use of arrays or data generated from an array. Data may be readily available. Moreover, an array may be prepared in order to generate data that may then be used in correlation studies.

An array generally refers to ordered macroarrays or microarrays of nucleic acid molecules (probes), such as the oligonucleotides of the disclosure. The nucleic acid molecules are positioned on a support material in a spatially separated organization. Macroarrays are typically sheets of nitrocellulose or nylon upon which nucleic acids have been spotted. Microarrays position the nucleic acid oligos more densely such that up to millions of nucleic acid molecules can be fit into a region typically 1 to 4 square centimeters. Microarrays can be fabricated by spotting nucleic acid molecules, e.g., genes, oligonucleotides, etc., onto substrates or fabricating oligonucleotide sequences in situ on a substrate. Spotted or fabricated nucleic acid molecules can be applied in a high density matrix pattern of up to about 30 non-identical nucleic acid molecules per square centimeter or higher, e.g. up to about 100 or even 1000 per square centimeter. Microarrays typically use coated glass as the solid support, in contrast to the nitrocellulose-based material of filter arrays. By having an ordered array of complementing nucleic acid samples, the position of each sample can be tracked and linked to the original sample. A variety of different array devices in which a plurality of distinct nucleic acid oligos are stably associated with the surface of a solid support are known to those of skill in the art. Useful substrates for arrays include nylon, glass and silicon. Such arrays may vary in a number of different ways, including average probe length, sequence or types of oligos, nature of bond between the probe and the array surface, e.g. covalent or non-covalent, and the like.

Representative methods and apparatus for preparing a microarray have been described, for example, in U.S. Pat. Nos. 5,143,854; 5,202,231; 5,242,974; 5,288,644; 5,324,633; 5,384,261; 5,405,783; 5,412,087; 5,424,186; 5,429,807; 5,432,049; 5,436,327; 5,445,934; 5,468,613; 5,470,710; 5,472,672; 5,492,806; 5,525,464; 5,503,980; 5,510,270; 5,525,464; 5,527,681; 5,529,756; 5,532,128; 5,545,531; 5,547,839; 5,554,501; 5,556,752; 5,561,071; 5,571,639; 5,580,726; 5,580,732; 5,593,839; 5,599,695; 5,599,672; 5,610,287; 5,624,711; 5,631,134; 5,639,603; 5,654,413; 5,658,734; 5,661,028; 5,665,547; 5,667,972; 5,695,940; 5,700,637; 5,744,305; 5,800,992; 5,807,522; 5,830,645; 5,837,196; 5,871,928; 5,847,219; 5,876,932; 5,919,626; 6,004,755; 6,087,102; 6,368,799; 6,383,749; 6,617,112; 6,638,717; 6,720,138, as well as WO 93/17126; WO 95/11995; WO 95/21265; WO 95/21944; WO 95/35505; WO 96/31622; WO 97/10365; WO 97/27317; WO 99/35505; WO 09923256; WO 09936760; WO0138580; WO 0168255; WO 03020898; WO 03040410; WO 03053586; WO 03087297; WO 03091426; WO03100012; WO 04020085; WO 04027093; EP 373 203; EP 785 280; EP 799 897 and UK 8 803 000; the disclosures of which are all herein incorporated by reference.

It is contemplated that the arrays can be high density arrays, such that they contain 100 or more different oligos. It is contemplated that they may contain 1000, 16,000, 65,000, 250,000 or 1,000,000 or more different oligos (or any range derivable therein).

The location and sequence of each different oligo sequence in the array are generally known. Moreover, the large number of different oligos can occupy a relatively small area providing a high density array having a probe density of generally greater than about 60, 100, 600, 1000, 5,000, 10,000, 40,000, 100,000, or 400,000 different oligonucleotide probes per cm2. The surface area of the array can be about or less than about 1, 1.6, 2, 3, 4, 5, 6, 7, 8, 9, or 10 cm2.

Moreover, a person of ordinary skill in the art could readily analyze data generated using an array. Such protocols include information found in WO 9743450; WO 03023058; WO 03022421; WO 03029485; WO 03067217; WO 03066906; WO 03076928; WO 03093810; WO 03100448A1, all of which are specifically incorporated by reference.

In embodiments of the disclosure, a composition comprising transposome complexes, wherein each complexes comprises a first barcode, may be transferred into a first cell; a composition comprising transposome complexes, wherein each complexes comprises a second barcode, may be transferred into a second cell; a composition comprising transposome complexes, wherein each complexes comprises a third barcode, may be transferred into a third cell; a composition comprising transposome complexes, wherein each complexes comprises a fourth barcode, may be transferred into a fourth cell; a composition comprising transposome complexes, wherein each complexes comprises a fifth barcode, may be transferred into a fifth cell; a composition comprising transposome complexes, wherein each complexes comprises a sixth barcode, may be transferred into a sixth cell; and a composition comprising transposome complexes, wherein each complexes comprises a nth barcode, may be transferred into a nth cell. N may be a number from 1-1000000 or at most or at least 10, 50, 75, 100, 500, 1000, 5000, 10000, 15000, 20000, 25000, 50000, 75000, 100000, 125000, 150000, 175000, 200000, 250000, 300000, 350000, 400000, 450000, 500000, 550000, 600000, 700000, 800000, 900000, or 1000000 cells (or any derivable range therein).

### III. Methods of Analyzing Nucleic Acids

### A. Single-Cell Analysis Techniques

### 1. Drop-Seq

Drop-Seq analyzes mRNA transcripts from droplets of individual cells in a highly parallel fashion. This single-cell sequencing method uses a microfluidic device to compartmentalize droplets containing a single cell, lysis buffer, and a microbead covered with barcoded primers. Each primer contains: 1) a 30 bp oligo(dT) sequence to bind mRNAs; 2) an 8 bp molecular index to identify each mRNA strand uniquely; 3) a 12 bp barcode unique to each cell and 4) a universal sequence identical across all beads. Following compartmentalization, cells in the droplets are lysed and the released mRNA hybridizes to the oligo(dT) tract of the primer beads. Next, all droplets are pooled and broken to release the beads within. After the beads are isolated, they are reverse-transcribed with template switching. This generates the first cDNA strand with a PCR primer sequence in place of the universal sequence. cDNAs are PCR-amplified, and sequencing adapters are added using the Nextera XT Library Preparation Kit. The barcoded mRNA samples are ready for sequencing. This method is further described in Macosko, Evan Z., et al., Cell, 2015. 161(5): p. 1202-1214, which is herein incorporated by reference.

### 2. inDrop

inDrop is used for high-throughput single-cell labeling. This approach is similar to Drop-seq, but it uses hydrogel microspheres to introduce the oligonucleotides. Single cells from a cell suspension are isolated into droplets containing lysis buffer. After cell lysis, cell droplets are fused with a hydrogel microsphere containing cell-specific barcodes and another droplet with enzymes for RT. Droplets from all the wells are pooled and subjected to isothermal reactions for RT. The barcodes anneal to poly(A)+ mRNAs and act as primers for reverse transcriptase. Now that each mRNA strand has cell-specific barcodes, the droplets are pooled and broken, and the cDNA is purified. The 3′ ends of the cDNA strands are ligated to adapters, amplified, annealed to indexed primers, and amplified further before sequencing. This method is further described in Klein, Allon M., et al., Cell, 2015. 161(5): p. 1187-1201, which is herein incorporated by reference.

### 3. CEL-seq

CEL-Seq uses barcoding and pooling of RNA to overcome challenges from low input. In this method, each cell undergoes RT with a unique barcoded primer in its individual tube. After second-strand synthesis, cDNAs from all reaction tubes are pooled and PCR-amplified. Paired-end deep sequencing of the PCR products allows for accurate detection of sequence information derived from both strands. This method, and related CEL-seq2 are further described in Hashimshony, T., et al., Cell Reports, 2012. 2(3): p. 666-673 and Hashimshony, T., et al., Genome Biology, 2016. 17(1): p. 77, which are herein incorporated by reference.

### 4. Quartz-Seq

The Quartz-Seq method optimizes whole-transcript amplification (WTA) of single cells. In this method, an RT primer with a T7 promoter and PCR target is first added to the extracted mRNA. RT synthesizes first-strand cDNA, after which the RT primer is digested by exonuclease I. Next, a poly(A) tail is added to the 3′ ends of first-strand cDNA, along with a poly(dT) primer containing a PCR target. After second-strand generation, a blocking primer is added to ensure PCR enrichment in sufficient quantity for sequencing. Deep sequencing allows for accurate, high-resolution representation of the whole transcriptome of a single cell.

### 5. MARS-Seq

MARS-Seq profiles the transcriptional dynamics of single cells in an automated and massively parallel workflow with high resolution. MARS-Seq can be used with in vivo samples containing a wide variety of different cell subpopulations. Single cells are first isolated into individual wells using FACS. Each cell is lysed, and the 3′ ends of mRNAs are annealed to unique molecular identifiers containing a T7 promoter. The mRNA is reverse-transcribed to generate the first cDNA strand and treated with exonuclease I to remove leftover RT primers. Next, the cellular lysates are pooled together and converted to double-stranded cDNA. The DNA strands are transcribed to RNA and treated with DNase to remove leftover DNA templates in the mixture. The RNA strands are fragmented and annealed to sequencing adapters, followed by RT to generate barcoded cDNA libraries that are ready for sequencing.

### 6. CytoSeq

CytoSeq enables gene expression profiling of thousands of single cells. In this method, single cells are randomly deposited into wells. A combinatorial library of beads with specific capture probes is added to each well. After cell lysis, mRNAs hybridize the to beads, which are pooled subsequently for RT, amplification, and sequencing. Deep sequencing provides accurate, high-coverage gene expression profiles of several single cells.

### 7. Hi-SCL

Hi-SCL generates transcriptome profiles for thousands of single cells using a custom microfluidics system, similar to Drop-Seq and inDrop. Single cells from cell suspension are isolated into droplets containing lysis buffer. After cell lysis, cell droplets are fused with a droplet containing cell-specific barcodes and another droplet with enzymes for RT. The droplets from all the wells are pooled and subjected to isothermal reactions for RT. The barcodes anneal to poly(A)+ mRNAs and act as primers for reverse transcriptase. Now that each mRNA strand has cell-specific barcodes, the droplets are broken, and the cDNA is purified. The 3′ ends of the cDNA strands are ligated to adapters, amplified, annealed to indexed primers, and amplified further before sequencing.

### 8. Seq-Well

Single-cell RNA-seq can precisely resolve cellular states, but applying this method to low-input samples is challenging. Here, the inventors present Seq-Well, a portable, low-cost platform for massively parallel single-cell RNA-seq. Barcoded mRNA capture beads and single cells are sealed in an array of subnanoliter wells using a semipermeable membrane, enabling efficient cell lysis and transcript capture. This method is further described in Gierahn et al., Nat Methods. 2017 April; 14(4):395-398, which is herein incorporated by reference. This method is further described in Gierahn, T. M., et al., Nature Methods, 2017. 14: p. 395, which is herein incorporated by reference.

### 9. Microwell-Seq

Microwell-seq confines single cells and barcoded poly(dT) mRNA capture beads in a PDMS array of subnanoliter wells. Well dimensions are designed to accommodate only one bead. Cells are loaded by gravity with a rate of dual occupancy that can be tuned by adjusting the number of cells and loaded and visualized prior to processing. This method is further described in Han, X., et al., Cell, 2018. 172(5): p.1091-1107.e17, which is herein incorporated by reference.

### 10. Nanogrid-Seq

Nanogrid-seq is a nanogrid platform and microfluidic depositing system that enables imaging, selection, and sequencing of thousands of single cells or nuclei in parallel. This method is further described in Gao, R., et al., Nature Communications, 2017. 8(1): p. 228, which is herein incorporated by reference.

### 11. Sci-Seq

Sci-seq refers to Single cell Combinatorial Indexed Sequencing (SCI-seq) that can be used as a means of simultaneously generating thousands of low-pass single cell libraries for somatic copy number variant detection. This is further described in Vitak, S. A., et al., Nature Methods, 2017. 14: p. 302, which is herein incorporated by reference.

### 12. Direct-Tagmentation

Enzymes called transposases randomly cut the DNA into short segments (“tags”). Adapters are added on either side of the cut points (ligation). Strands that fail to have adapters ligated are washed away. The adaptors may contain barcodes and/or primer binding sites for detection and amplification of the genomic sequences. This is further described in Zahn, H., et al., Nature Methods, 2017. 14: p. 167, which is herein incorporated by reference.

### 13. sciATAC-Seq

sci-ATAC-seq is a single-cell ATAC-seq protocol. This technique can be used to determine chromatin accessibility both between and within populations of single cells. Single-cell ATAC-Seq relies on combinatorial cellular indexing, and thus does not require the physical isolation of individual cells during library construction. The technique scales sublinearly in time and cost and can profile thousands of individual cells in a single experiment. This method is further described in Cusanovich, D. A., et al., Science, 2015. 348(6237): p. 910, which is herein incorporated by reference. A related method, nano-well scATAC-seq is described in Mezger, A., et al., High-throughput chromatin accessibility profiling at single-cell resolution, bioRxiv, 2018, which is incorporated by reference.

Other methods include 10× genomics RNA sequencing platform, described in Zheng, G. X. Y., et al., Nature Communications, 2017. 8: p. 14049; SMART-seq, described in Ramskold, D., et al., Nature Biotechnology, 2012. 30: p. 777; SMART-seq2, described in Picelli, S., et al., Nature Protocols, 2014. 9: p. 171, which are all herein incorporated by reference in their entirety. It is contemplated that embodiments in the disclosed references may be incorporated into embodiments described in this disclosure.

### B. Sequencing Methods

The methods of the disclosure may further include sequencing of nucleic acids to determine the identity/quantity of barcodes in a cell or cell population. The sequencing methods described below are exemplary methods that may be used in conjunction with the single cell analysis techniques described herein as well as the method embodiments of the disclosure.

### 2. Massively Parallel Signature Sequencing (MPSS)

The first of the next-generation sequencing technologies, massively parallel signature sequencing (or MPSS), was developed in the 1990s at Lynx Therapeutics. MPSS was a bead-based method that used a complex approach of adapter ligation followed by adapter decoding, reading the sequence in increments of four nucleotides. This method made it susceptible to sequence-specific bias or loss of specific sequences. Because the technology was so complex, MPSS was only performed ‘in-house’ by Lynx Therapeutics and no DNA sequencing machines were sold to independent laboratories. Lynx Therapeutics merged with Solexa (later acquired by Illumina) in 2004, leading to the development of sequencing-by-synthesis, a simpler approach acquired from Manteia Predictive Medicine, which rendered MPSS obsolete. However, the essential properties of the MPSS output were typical of later “next-generation” data types, including hundreds of thousands of short DNA sequences. In the case of MPSS, these were typically used for sequencing cDNA for measurements of gene expression levels. Indeed, the powerful Illumina HiSeq2000, HiSeq2500 and MiSeq systems are based on MPSS.

### 3. Polony Sequencing

The Polony sequencing method, developed in the laboratory of George M. Church at Harvard, was among the first next-generation sequencing systems and was used to sequence a full genome in 2005. It combined an in vitro paired-tag library with emulsion PCR, an automated microscope, and ligation-based sequencing chemistry to sequence an E. coli genome at an accuracy of >99.9999% and a cost approximately 1/9 that of Sanger sequencing. The technology was licensed to Agencourt Biosciences, subsequently spun out into Agencourt Personal Genomics, and eventually incorporated into the Applied Biosystems SOLiD platform, which is now owned by Life Technologies.

### 4. 454 Pyrosequencing

A parallelized version of pyrosequencing was developed by 454 Life Sciences, which has since been acquired by Roche Diagnostics. The method amplifies DNA inside water droplets in an oil solution (emulsion PCR), with each droplet containing a single DNA template attached to a single primer-coated bead that then forms a clonal colony. The sequencing machine contains many picoliter-volume wells each containing a single bead and sequencing enzymes. Pyrosequencing uses luciferase to generate light for detection of the individual nucleotides added to the nascent DNA, and the combined data are used to generate sequence read-outs. This technology provides intermediate read length and price per base compared to Sanger sequencing on one end and Solexa and SOLiD on the other.

### 5. Illumina (Solexa) Sequencing

Solexa, now part of Illumina, developed a sequencing method based on reversible dye-terminators technology, and engineered polymerases, that it developed internally. The terminated chemistry was developed internally at Solexa and the concept of the Solexa system was invented by Balasubramanian and Klennerman from Cambridge University's chemistry department. In 2004, Solexa acquired the company Manteia Predictive Medicine in order to gain a massively parallel sequencing technology based on “DNA Clusters”, which involves the clonal amplification of DNA on a surface. The cluster technology was co-acquired with Lynx Therapeutics of California. Solexa Ltd. later merged with Lynx to form Solexa Inc.

In this method, DNA molecules and primers are first attached on a slide and amplified with polymerase so that local clonal DNA colonies, later coined “DNA clusters”, are formed. To determine the sequence, four types of reversible terminator bases (RT-bases) are added and non-incorporated nucleotides are washed away. A camera takes images of the fluorescently labeled nucleotides, then the dye, along with the terminal 3′ blocker, is chemically removed from the DNA, allowing for the next cycle to begin. Unlike pyrosequencing, the DNA chains are extended one nucleotide at a time and image acquisition can be performed at a delayed moment, allowing for very large arrays of DNA colonies to be captured by sequential images taken from a single camera.

Decoupling the enzymatic reaction and the image capture allows for optimal throughput and theoretically unlimited sequencing capacity. With an optimal configuration, the ultimately reachable instrument throughput is thus dictated solely by the analog-to-digital conversion rate of the camera, multiplied by the number of cameras and divided by the number of pixels per DNA colony required for visualizing them optimally (approximately 10 pixels/colony). In 2012, with cameras operating at more than 10 MHz A/D conversion rates and available optics, fluidics and enzymatics, throughput can be multiples of 1 million nucleotides/second, corresponding roughly to one human genome equivalent at 1× coverage per hour per instrument, and one human genome re-sequenced (at approx. 30×) per day per instrument (equipped with a single camera).

### 6. Solid Sequencing

Applied Biosystems' (now a Life Technologies brand) SOLiD technology employs sequencing by ligation. Here, a pool of all possible oligonucleotides of a fixed length are labeled according to the sequenced position. Oligonucleotides are annealed and ligated; the preferential ligation by DNA ligase for matching sequences results in a signal informative of the nucleotide at that position. Before sequencing, the DNA is amplified by emulsion PCR. The resulting beads, each containing single copies of the same DNA molecule, are deposited on a glass slide. The result is sequences of quantities and lengths comparable to Illumina sequencing. This sequencing by ligation method has been reported to have some issue sequencing palindromic sequences.

### 7. Ion Torrent Semiconductor Sequencing

Ion Torrent Systems Inc. (now owned by Life Technologies) developed a system based on using standard sequencing chemistry, but with a novel, semiconductor based detection system. This method of sequencing is based on the detection of hydrogen ions that are released during the polymerization of DNA, as opposed to the optical methods used in other sequencing systems. A microwell containing a template DNA strand to be sequenced is flooded with a single type of nucleotide. If the introduced nucleotide is complementary to the leading template nucleotide it is incorporated into the growing complementary strand. This causes the release of a hydrogen ion that triggers a hypersensitive ion sensor, which indicates that a reaction has occurred. If homopolymer repeats are present in the template sequence multiple nucleotides will be incorporated in a single cycle. This leads to a corresponding number of released hydrogens and a proportionally higher electronic signal.

### 8. DNA Nanoball Sequencing

DNA nanoball sequencing is a type of high throughput sequencing technology used to determine the entire genomic sequence of an organism. The company Complete Genomics uses this technology to sequence samples submitted by independent researchers. The method uses rolling circle replication to amplify small fragments of genomic DNA into DNA nanoballs. Unchained sequencing by ligation is then used to determine the nucleotide sequence. This method of DNA sequencing allows large numbers of DNA nanoballs to be sequenced per run and at low reagent costs compared to other next generation sequencing platforms. However, only short sequences of DNA are determined from each DNA nanoball which makes mapping the short reads to a reference genome difficult. This technology has been used for multiple genome sequencing projects and is scheduled to be used for more.

### 9. Heliscope Single Molecule Sequencing

Heliscope sequencing is a method of single-molecule sequencing developed by Helicos Biosciences. It uses DNA fragments with added poly-A tail adapters which are attached to the flow cell surface. The next steps involve extension-based sequencing with cyclic washes of the flow cell with fluorescently labeled nucleotides (one nucleotide type at a time, as with the Sanger method). The reads are performed by the Heliscope sequencer. The reads are short, up to 55 bases per run, but recent improvements allow for more accurate reads of stretches of one type of nucleotides. This sequencing method and equipment were used to sequence the genome of the M13 bacteriophage.

### 10. Single Molecule Real Time (SMRT) Sequencing

SMRT sequencing is based on the sequencing by synthesis approach. The DNA is synthesized in zero-mode wave-guides (ZMWs)—small well-like containers with the capturing tools located at the bottom of the well. The sequencing is performed with use of unmodified polymerase (attached to the ZMW bottom) and fluorescently labelled nucleotides flowing freely in the solution. The wells are constructed in a way that only the fluorescence occurring by the bottom of the well is detected. The fluorescent label is detached from the nucleotide at its incorporation into the DNA strand, leaving an unmodified DNA strand. According to Pacific Biosciences, the SMRT technology developer, this methodology allows detection of nucleotide modifications (such as cytosine methylation). This happens through the observation of polymerase kinetics. This approach allows reads of 20,000 nucleotides or more, with average read lengths of 5 kilobases.

### C. Molecular Biology Techniques

Embodiments of the disclosure relate to oligonucleotides, transposases, library construction, sequencing, and determining RNA and/or DNA profiles in cells. Methods of the disclosure may include molecular biology techniques such polymerase chain reaction (PCR), real-time-PCR, reverse transcription, reverse transcription-PCR, northern blot, western blot, in situ hybridization, Southern blot, slot-blotting, nuclease protection assay and oligonucleotide arrays.

In certain aspects, RNA isolated from cells can be amplified to cDNA or cRNA before detection and/or quantitation. The isolated RNA can be either total RNA or mRNA. The RNA amplification can be specific or non-specific. In some embodiments, the amplification is specific in that it specifically amplifies barcodes that identify a spatial characteristic and/or barcodes that identify cellular nucleic acids. In some embodiments, random primers are utilized. In some embodiments, the amplification and/or reverse transcriptase step includes random priming. Suitable amplification methods include, but are not limited to, reverse transcriptase PCR, isothermal amplification, ligase chain reaction, and Qbeta replicase. The amplified nucleic acid products can be detected and/or quantitated through hybridization to labeled probes. In some embodiments, detection may involve fluorescence resonance energy transfer (FRET) or some other kind of quantum dots.

Amplification primers or hybridization probes can be prepared from the nucleic acid sequence of a target region or of a primer binding site described herein. The term “primer” or “probe” as used herein, is meant to encompass any nucleic acid that is capable of priming the synthesis of a nascent nucleic acid in a template-dependent process. Typically, primers are oligonucleotides from ten to twenty and/or thirty base pairs in length, but longer sequences can be employed. Primers may be provided in double-stranded and/or single-stranded form, although the single-stranded form is preferred. The primer or probe may have a tale region that does not have base complementarity to a oligonucleotide of the disclosure. The tale region may be used to introduce additional sequences that facilitate the cloning and/or library construction of nucleic acids.

The use of a probe or primer of between 13 and 100 nucleotides, particularly between 17 and 100 nucleotides in length, or in some aspects up to 1-2 kilobases or more in length, allows the formation of a duplex molecule that is both stable and selective. Molecules having complementary sequences over contiguous stretches greater than 20 bases in length may be used to increase stability and/or selectivity of the hybrid molecules obtained. One may design nucleic acid molecules for hybridization having one or more complementary sequences of 20 to 30 nucleotides, or even longer where desired. Such fragments may be readily prepared, for example, by directly synthesizing the fragment by chemical means or by introducing selected sequences into recombinant vectors for recombinant production.

In one embodiment, each probe/primer comprises at least 15 nucleotides. For instance, each probe can comprise at least or at most 20, 25, 50, 75, 100, 125, 150, 175, 200, 225, 250, 275, 300, 325, 350, 400 or more nucleotides (or any range derivable therein). They may have these lengths and have a sequence that is identical or complementary to a gene described herein. Particularly, each probe/primer has relatively high sequence complexity and does not have any ambiguous residue (undetermined “n” residues). The probes/primers can hybridize to the target gene, including its RNA transcripts, under stringent or highly stringent conditions.

For applications requiring high selectivity, one will typically desire to employ relatively high stringency conditions to form the hybrids. For example, relatively low salt and/or high temperature conditions, such as provided by about 0.02 M to about 0.10 M NaCl at temperatures of about 50° C. to about 70° C. Such high stringency conditions tolerate little, if any, mismatch between the probe or primers and the template or target strand and would be particularly suitable for isolating specific genes or for detecting specific mRNA transcripts. It is generally appreciated that conditions can be rendered more stringent by the addition of increasing amounts of formamide.

In one embodiment, quantitative RT-PCR (such as TaqMan, ABI) is used for detecting and comparing the levels of RNA transcripts in samples. Quantitative RT-PCR involves reverse transcription (RT) of RNA to cDNA followed by relative quantitative PCR (RT-PCR). The concentration of the target DNA in the linear portion of the PCR process is proportional to the starting concentration of the target before the PCR was begun. By determining the concentration of the PCR products of the target DNA in PCR reactions that have completed the same number of cycles and are in their linear ranges, it is possible to determine the relative concentrations of the specific target sequence in the original DNA mixture. If the DNA mixtures are cDNAs synthesized from RNAs isolated from different tissues or cells, the relative abundances of the specific mRNA from which the target sequence was derived may be determined for the respective tissues or cells. This direct proportionality between the concentration of the PCR products and the relative mRNA abundances is true in the linear range portion of the PCR reaction. The final concentration of the target DNA in the plateau portion of the curve is determined by the availability of reagents in the reaction mix and is independent of the original concentration of target DNA. Therefore, the sampling and quantifying of the amplified PCR products may be carried out when the PCR reactions are in the linear portion of their curves. In addition, relative concentrations of the amplifiable cDNAs may be normalized to some independent standard, which may be based on either internally existing RNA species or externally introduced RNA species. The abundance of a particular mRNA species may also be determined relative to the average abundance of all mRNA species in the sample.

In one embodiment, the PCR amplification utilizes one or more internal PCR standards. The internal standard may be an abundant housekeeping gene in the cell or it can specifically be GAPDH, GUSB and β-2 microglobulin. These standards may be used to normalize expression levels so that the expression levels of different gene products can be compared directly. A person of ordinary skill in the art would know how to use an internal standard to normalize expression levels.

A problem inherent in some samples is that they are of variable quantity and/or quality. This problem can be overcome if the RT-PCR is performed as a relative quantitative RT-PCR with an internal standard in which the internal standard is an amplifiable cDNA fragment that is similar or larger than the target cDNA fragment and in which the abundance of the mRNA encoding the internal standard is roughly 5-100 fold higher than the mRNA encoding the target. This assay measures relative abundance, not absolute abundance of the respective mRNA species.

In another embodiment, the relative quantitative RT-PCR uses an external standard protocol. Under this protocol, the PCR products are sampled in the linear portion of their amplification curves. The number of PCR cycles that are optimal for sampling can be empirically determined for each target cDNA fragment. In addition, the reverse transcriptase products of each RNA population isolated from the various samples can be normalized for equal concentrations of amplifiable cDNAs.

### IV. Cells

As used herein, the terms “cell,” “cell line,” and “cell culture” may be used interchangeably. In some embodiments, the methods relate to a population of cells. A population of cells may be a collection of cells from a patient, from a particular tissue, or from a particular treatment condition. The population of cells may be of one cell type or of multiple cell types. Typically, a population of cells will have at least one cellular characteristic in common. All of these terms also include both freshly isolated cells and in vitro cultured or expanded cells. All of these terms also include their progeny, which is any and all subsequent generations. It is understood that all progeny may not be identical due to deliberate or inadvertent mutations. In the context of expressing a heterologous nucleic acid sequence, a “host cell” or simply a “cell” refers to a prokaryotic or eukaryotic cell, and it includes any transformable organism that is capable of replicating a vector or expressing a heterologous gene encoded by a vector or integrated nucleic acid. A host cell can, and has been, used as a recipient for vectors, viruses, and nucleic acids. A host cell may be “transfected” or “transformed,” which refers to a process by which exogenous nucleic acid, such as a recombinant protein-encoding sequence, is transferred or introduced into the host cell. A transformed cell includes the primary subject cell and its progeny.

In some embodiments, the cell is a eukaryotic cell. In some embodiments, the cell is an animal cell. In some aspects the cells of the disclosure are human cells. In other aspects the cells of the disclosure are an animal cell. In some aspects the cell or cells are diseased cells, cancer cells, tumor cells, immortalized cells, or cells isolated from a mammal. In further aspects, the cells represent a disease-model cell. In certain aspects the cells can be A549, B-cells, B16, BHK-21, C2C12, C6, CaCo-2, CAP/, CAP-T, CHO, CHO2, CHO-DG44, CHO-K1, COS-1, Cos-7, CV-1, Dendritic cells, DLD-1, Embryonic Stem (ES) Cell or derivative, H1299, HEK, 293, 293T, 293FT, Hep G2, Hematopoietic Stem Cells, HOS, Huh-7, Induced Pluripotent Stem (iPS) Cell or derivative, Jurkat, K562, L5278Y, LNCaP, MCF7, MDA-MB-231, MDCK, Mesenchymal Cells, Min-6, Monocytic cell, Neuro2a, NIH 3T3, NIH3T3L1, K562, NK-cells, NS0, Panc-1, PC12, PC-3, Peripheral blood cells, Plasma cells, Primary Fibroblasts, RBL, Renca, RLE, SF21, SF9, SH-SYSY, SK-MES-1, SK-N-SH, SL3, SW403, Stimulus-triggered Acquisition of Pluripotency (S TAP) cell or derivate SW403, T-cells, THP-1, Tumor cells, U20S, U937, peripheral blood lymphocytes, expanded T cells, hematopoietic stem cells, or Vero cells. In some embodiments, the cells are primary cells. In some embodiments, the cells are fixed, such as formalin-fixed. In some embodiments, the cells are in an endogenous location.

The term “passaged,” as used herein, is intended to refer to the process of splitting cells in order to produce large number of cells from pre-existing ones. Cells may be passaged multiple times prior to or after any step described herein. Passaging involves splitting the cells and transferring a small number into each new vessel. For adherent cultures, cells first need to be detached, commonly done with a mixture of trypsin-EDTA. A small number of detached cells can then be used to seed a new culture, while the rest is discarded. Also, the amount of cultured cells can easily be enlarged by distributing all cells to fresh flasks. Cells may be kept in culture and incubated under conditions to allow cell replication. In some embodiments, the cells are kept in culture conditions that allow the cells to under 1, 2, 3, 4, 5, 6, 7, 8, 9, 10 or more rounds of cell division.

In some embodiments, cells may subjected to limiting dilution methods to enable the expansion of clonal populations of cells. The methods of limiting dilution cloning are well known to those of skill in the art. Such methods have been described, for example for hybridomas but can be applied to any cell. Such methods are described in (Cloning hybridoma cells by limiting dilution, Journal of tissue culture methods, 1985, Volume 9, Issue 3, pp 175-177, by Joan C. Rener, Bruce L. Brown, and Roland M. Nardone) which is incorporated by reference herein.

Methods of the disclosure include the culturing of cells. Methods of culturing suspension and adherent cells are well-known to those skilled in the art. In some embodiments, cells are cultured in suspension, using commercially available cell-culture vessels and cell culture media. Examples of commercially available culturing vessels that may be used in some embodiments including ADME/TOX Plates, Cell Chamber Slides and Coverslips, Cell Counting Equipment, Cell Culture Surfaces, Corning HYPERFlask Cell Culture Vessels, Coated Cultureware, Nalgene Cryoware, Culture Chamber, Culture Dishes, Glass Culture Flasks, Plastic Culture Flasks, 3D Culture Formats, Culture Multiwell Plates, Culture Plate Inserts, Glass Culture Tubes, Plastic Culture Tubes, Stackable Cell Culture Vessels, Hypoxic Culture Chamber, Petri dish and flask carriers, Quickfit culture vessels, Scale-Up Cell Culture using Roller Bottles, Spinner Flasks, 3D Cell Culture, or cell culture bags.

In other embodiments, media may be formulated using components well-known to those skilled in the art. Formulations and methods of culturing cells are described in detail in the following references: Short Protocols in Cell Biology J. Bonifacino, et al., ed., John Wiley & Sons, 2003, 826 pp; Live Cell Imaging: A Laboratory Manual D. Spector & R. Goldman, ed., Cold Spring Harbor Laboratory Press, 2004, 450 pp.; Stem Cells Handbook S. Sell, ed., Humana Press, 2003, 528 pp.; Animal Cell Culture: Essential Methods, John M. Davis, John Wiley & Sons, Mar. 16, 2011; Basic Cell Culture Protocols, Cheryl D. Helgason, Cindy Miller, Humana Press, 2005; Human Cell Culture Protocols, Series: Methods in Molecular Biology, Vol. 806, Mitry, Ragai R.; Hughes, Robin D. (Eds.), 3rd ed. 2012, XIV, 435 p. 89, Humana Press; Cancer Cell Culture: Method and Protocols, Cheryl D. Helgason, Cindy Miller, Humana Press, 2005; Human Cell Culture Protocols, Series: Methods in Molecular Biology, Vol. 806, Mitry, Ragai R.; Hughes, Robin D. (Eds.), 3rd ed. 2012, XIV, 435 p. 89, Humana Press; Cancer Cell Culture: Method and Protocols, Simon P. Langdon, Springer, 2004; Molecular Cell Biology. 4th edition, Lodish H, Berk A, Zipursky S L, et al., New York: W. H. Freeman; 2000, Section 6.2 Growth of Animal Cells in Culture, all of which are incorporated herein by reference.

### V. Kits

Certain aspects of the present disclosure also concern kits containing nucleic acids, vectors, transposase, molecular cloning and library construction reagents, and assay reagents. The kits may be used to implement the methods of the disclosure. In some embodiments, kits can be used to barcode eukaryotic cells. In certain embodiments, a kit contains, contains at least or contains at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 100, 500, 1,000 or more nucleic acid probes, oligos, primers, or synthetic RNA molecules, or any value or range and combination derivable therein. In some embodiments, universal probes or primers are included for amplifying, identifying, or sequencing a barcode. Such reagents may also be used to generate or test host cells that can be used in screens.

In certain embodiments, the kits may comprise materials for analyzing cell morphology and/or phenotype, such as histology slides and reagents, histological stains, alcohol, buffers, tissue embedding mediums, paraffin, formaldehyde, and tissue dehydrant.

Kits may comprise components, which may be individually packaged or placed in a container, such as a tube, bottle, vial, syringe, or other suitable container means.

Individual components may also be provided in a kit in concentrated amounts; in some embodiments, a component is provided individually in the same concentration as it would be in a solution with other components. Concentrations of components may be provided as 1×, 2×, 5×, 10×, or 20× or more.

Kits for using probes, polypeptide or polynucleotide detecting agents of the disclosure for drug discovery are contemplated.

In certain aspects, negative and/or positive control agents are included in some kit embodiments. The control molecules can be used to verify transfection efficiency and/or control for transfection-induced changes in cells.

Embodiments of the disclosure include kits for analysis of a pathological sample by assessing a nucleic acid or polypeptide profile for a sample comprising, in suitable container means, two or more RNA probes or primers for detecting expressed polynucleotides. Furthermore, the probes or primers may be labeled. Labels are known in the art and also described herein. In some embodiments, the kit can further comprise reagents for labeling probes, nucleic acids, and/or detecting agents. The kit may also include labeling reagents, including at least one of amine-modified nucleotide, poly(A) polymerase, and poly(A) polymerase buffer. Labeling reagents can include an amine-reactive dye. Kits can comprise any one or more of the following materials: enzymes, reaction tubes, buffers, detergent, primers, probes, antibodies. In some embodiments, these kits include the needed apparatus for performing RNA extraction, RT-PCR, and gel electrophoresis. Instructions for performing the assays can also be included in the kits.

The kits may further comprise instructions for using the kit for assessing expression, means for converting the expression data into expression values and/or means for analyzing the expression values or sequence data.

Kits may comprise a container with a label. Suitable containers include, for example, bottles, vials, and test tubes. The containers may be formed from a variety of materials such as glass or plastic. The container may hold a composition which includes a probe that is useful for the methods of the disclosure. The kit may comprise the container described above and one or more other containers comprising materials desirable from a commercial and user standpoint, including buffers, diluents, filters, needles, syringes, and package inserts with instructions for use.

### VI. Examples

The following examples are included to demonstrate preferred embodiments of the disclosure. It should be appreciated by those of skill in the art that the techniques disclosed in the examples which follow represent techniques discovered by the inventor to function well in the practice of the disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

### Example 1—Spatial Nucleus Barcoding (SNUBAR

### A. Overview of Single Nucleus Spatial Barcode Sequencing

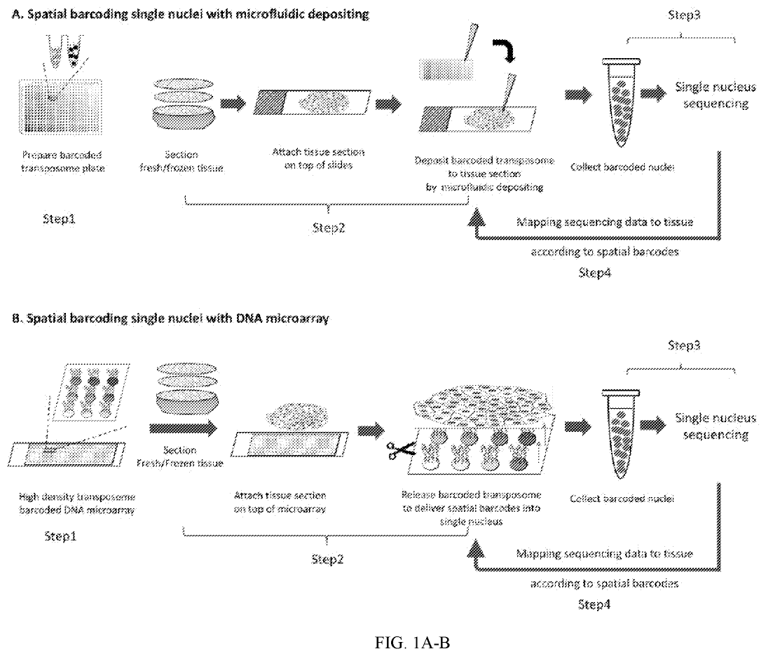

The fundamental principle of SNUBAR is to perform spatial barcoding of single nuclei across tissue sections in situ (before tissue dissocation), after which the nuclei with spatial barcodes are released and pooled to perform existing high-throughput single cell sequencing methods. SNUBAR can be performed using two different experimental approaches. In the first approach (FIG. 1A), the inventors assemble a series (eg. 96-1536) of different transposome complexes that each contain a unique spatial barcode oligonucleotide adapter and a Tn5 transposase complex. The inventors then permeabilize the tissue and microdeposit the transposomes with the spatial barcodes across different regions of the tissue section, which can be accomplished with different techniques (eg. micropipetting, acoustic liquid transfer). The barcoded nuclei are then scrapped from the slide or dissociated from tissue and pooled together into a suspension for single cell sequencing. After single cell sequencing the positional indexes from each nucleus/cell are used to identify the original spatial coordinates of the cells in the tissue sections. The second approach (FIG. 1B) involves first synthesizing a custom microarray that contains pre-printed spatial barcode oligonucleotide adapters across thousands of features. Tissue sections are then placed directly on top of the microarray and permeabilized to release the spatial barcode adapters that are subsequently incorporated into the transposome and delivered into single nuclei across the tissue section. The nuclei are then scrapped from the microarray and pooled for high-throughput single cell sequencing methods, after which the spatial index is used to identify the original position of the cell in the tissue.

### B. Spatial Barcode Oligonucleotide Adapter Structure

To deliver spatial barcodes to each cell in a tissue section, the inventors developed a transposome barcoding system. This system consists of spatial barcode oligonucleotide adapters and a transposome or transposase. The molecular structure of each spatial barcode oligonucleotide adapter is composed of three parts (FIG. 2A). The first part is either a sequence that binds directly to the transposase enzyme, or (FIG. 2A) a sequence that binds to complementary universal oligonucleotide adapters in the transposome (referred to herein as a transposome adaptor region). The second part is a spatial barcode sequence that can be any size of nucleotides (eg. 8-18 bp), referred to herein as a barcode region, which are assigned to different cells or regions in tissue sections to barcode nuclei. This sequence may also include a molecular barcode (MI) which can be used to count how many barcode sequences are delivered into each cell or nucleus. The third component is platform-specific sequence that are used for amplification of DNA or RNA or for binding by down-stream single cell sequencing methods (referred to herein as the target region). The platform-specific sequence acts as a target for the subsequent binding and amplification of the downstream library preparation chemistry. For example, if the barcoded single nucleus will be sequenced by high-throughput 3′ single cell RNA sequencing (Drop-seq) the library specific sequence would be a PCR handle sequence and a polyA sequence, PCR handle sequence will be used as PCR primer binding sequence to amplify and sequence spatial barcode sequence, while the polyA sequence can be bind by polyT oligonucleotides barcoded beads and transcript by reverse transcriptase (FIG. 2A). As another example, if the barcoded nuclei will be sequenced by high-throughput single cell DNA sequencing for copy number (eg. direct tagmentation based chemistry), the library specific sequences will be universal sequences, where the universal sequence will be used to mark spatial barcode positions. Although the inventors provide only two examples here, the spatial barcode adapter sequence can be customized based on different downstream sequencing library construction methods and applications.

### C. Assembly of the Spatial Index Transposome

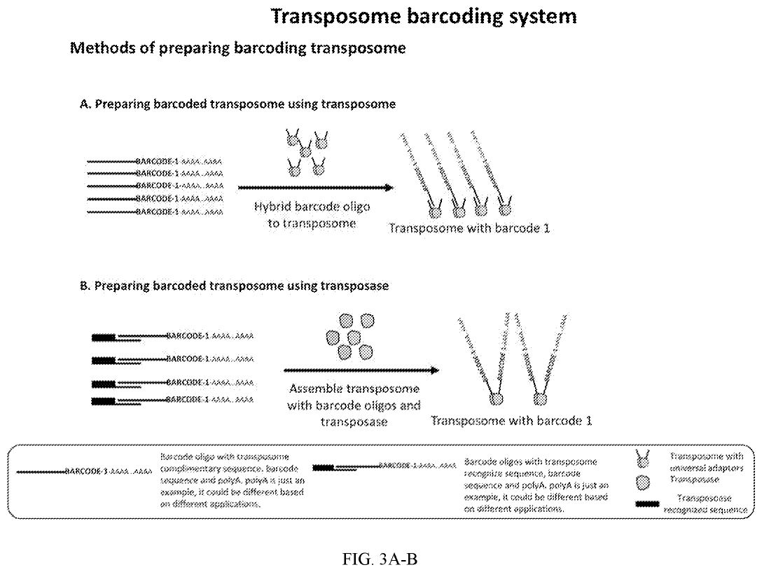

The spatial barcodes can either be assembled into an existing Tn5 transposome with universal adapters (eg. Illumina Tn5 transposome—TDE1 in Nextera DNA Library Prep Kit) or can be incorporated into a Tn5 transposase enzyme that does not have any oligonucleotides incorporated (FIG. 3). To assemble the spatial transposome barcoding system, the inventors first combine the spatial barcode oligos, with universal adaptors (such as Illumina Tn5 transposome (TDE1 in Nextera DNA Library Prep Kit or ATM in Nextera XT DNA Library Prep Kit)), and hybridize the barcode oligos or probes to the Illumina transposome to produce final barcoded transposome (FIG. 3A). Alternatively, the barcode oligos or probes can be used with transposase recognize sequences and binding them to naked transposase (eg. EZ-Tn5™ Transposase, Lucigen or MuA Transposase, Thermo Scientific™) to assemble the spatial barcoded transposome (FIG. 3B).

### D. Delivery of Spatial Index Transposome to Single Nuclei in Tissues

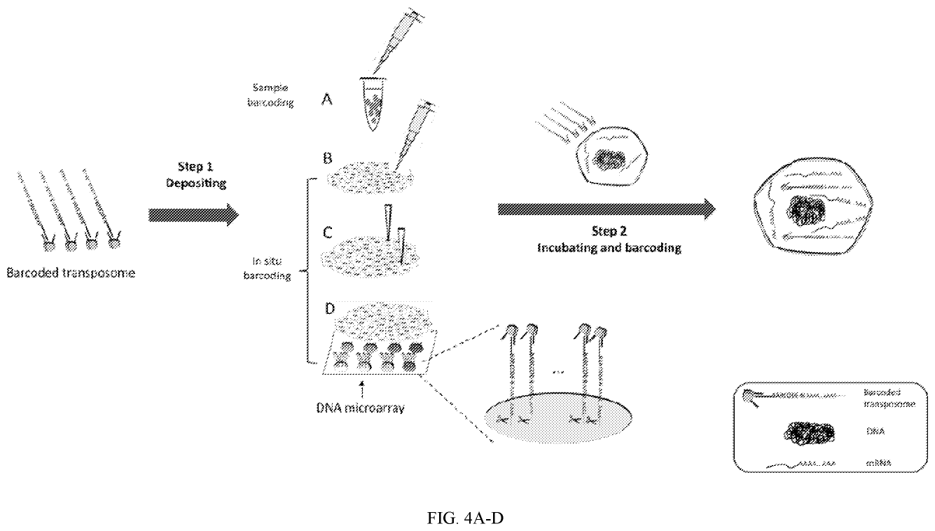

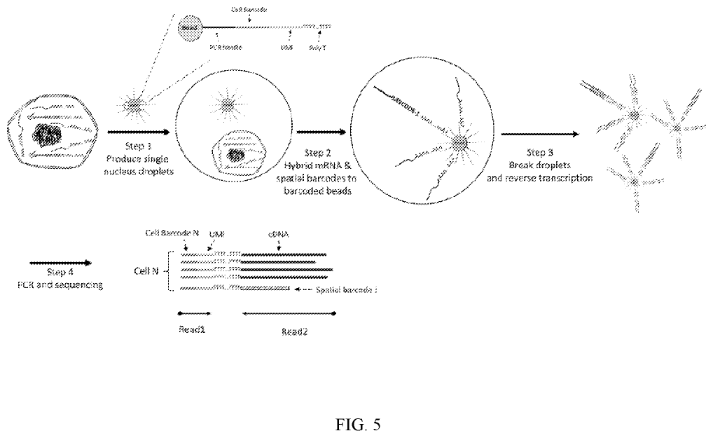

Several different approaches can be used to deliver spatial barcode to each single nucleus in a tissue section with the spatial barcode transposome system. The simplest approach involves using manual micro-pipetting, in which the different barcoded transposome reagents (1 barcode per transposome complex) is pipetted on top of each single nucleus or gasket well, with the aid of a microscope. After incubating with nuclei, the barcoded transposome will enter the nuclear membrane and deliver the spatial barcode into the nucleus, (FIG. 4B). Alternative variations of this approach that are more high-throughput, include using a microfluidic depositing system (microarray printer or liquid transfer system) to deliver the transposome complex across a tissue section in defined spatial regions (FIG. 4C). A different approach that enables barcoding of thousands to tens of thousands of spatial regions, involves designing a custom barcoded DNA microarray. In this customized microarray the barcode oligos or probes are printed on the surface of the DNA microarray, and are used to load a transposome with universal adaptors (eg. Illumina Tn5 transposome (TDE1 in Nextera DNA Library Prep Kit or ATM in Nextera XT DNA Library Prep Kit)) or transposase (eg Tn5, MuA) to the DNA microarray (FIG. 4D). After loading the transposome on the microarray, fresh or frozen tissue section are loaded on top of the barcoded transposome microarray. The tissue is then permeabilized followed by releasing the barcoded transposome on the microarray. The transposome will deliver the spatial barcode go into each nucleus across the tissue section.

### E. Single Cell/Nucleus Sequencing Library Preparation and Sequencing of Spatial Barcoded Nuclei

After the spatial barcodes are delivered into the nuclei, the nuclei can be used to prepare different single cell sequencing libraries, for example single cell RNA- seq, single cell DNA-seq, single cell ATAC-seq et. al, depends on different aims. Delivered spatial barcodes act as a molecular target for whole-genome-amplifications, whole-transcriptome amplification or tagmentation based chemistries for amplification and library construction chemistry. For example, if the spatial barcoded nuclei will be used for high throughput single cell mRNA sequencing (eg. Drop-seq), load spatial barcoded single nucleus (poly A tailed, eg. FIG. 2A) together with barcoded beads and oil to form single nuclei droplets (FIG. 5 step 1), the nucleus is lysed and release its mRNA and spatial barcode, which will further hybridize to the polyT primers on the surface of barcoded bead (FIG. 5 step 2). Then break the droplets, collect beads and do reverse transcription with template switching oligos (FIG. 5, step 3). PCR product is collected and sequencing, FIG. 5 shows an example that using Illumina paired end sequencing to sequence library of spatial barcoded single nucleus, read 1 will sequence the cell barcode and UMI, and read 2 will sequence the cDNA or spatial barcode. In one barcoded nucleus, all cDNA and spatial will carry same cell barcodes, this information will be used to address the real position of the nuclei. Besides preparation Drop-seq library, spatial barcoded nuclei also can be sequenced by other single cell RNA sequencing methods, such as SMART-seq-based, MARS-seq based, CEL-seq based, Drop-seq based methods such as 10× Genomics. In addition, slightly modify the spatial barcode sequences, the spatial barcode nuclei can be easily adapted for DNA and epigenomic amplification chemistries, such as for single cell DNA sequencing, include MDA, DOP-PCR, MALBAC, LIANTI or tagmentation based chemistries; for epigenomic methods, ATAC-seq and methylome sequencing et. al. Downstream sequencing platforms can include first generation sequencers (eg. sanger sequencing), next-generation sequencing platforms (Illumina, Ion Torrent, 454 sequencing, ABI), or third-generation single molecule sequencing platforms (PacBio's SMRT sequencing, Oxford Nanopore's Nanopore sequencing).

### F. Mapping of Spatial Barcode and Single Cell Genomic Libraries after Sequencing

After sequencing is completed, the final step involves demultiplexing the spatial barcodes and cell barcodes, as well as the genomic data. The spatial barcodes may be prepared in a separate sequencing library (for example for RNA) or may be part of the same sequencing library that includes the cell barcodes and genomic datasets (eg. for DNA). When the spatial barcodes are constructed as part of a separate library, the spatial barcodes also share the same ‘cell barcodes’ as the genomic data, which are used to match the spatial positions to the genomic datasets. For example, if single cell RNA sequencing is performed using SNUBAR and the 10× genomics Chromium 3′ single cell RNA reagent kit, after cDNA amplification the spatial barcode sequence (<100 bp) is much shorter than the cDNA size (>1 k bp) and is separated by size selection to prepare two independent sequencing libraries (with the same cell barcodes). Since the spatial barcode library is physically separated from the genomic library (cDNA), the barcodes can be identified after next-generation sequencing (Read 1 are cell barcodes, reads 2 are spatial barcode and poly dA sequence). Another example is SNUBAR and single cell DNA sequencing using direct tagmentation chemistry, in which the spatial barcode will be delivered into nuclei with the assistant of transposome, after which the spatial barcode library is sequenced together with genomic DNA library (since barcode library size is only a little bit smaller than gDNA library). For the DNA libraries, spatial barcodes are recovered by using specific sequences or sequence composition structure in the designed spatial barcode adapters.

### G. Transposome Barcoding System for Sample Barcoding

Another application of the transposome barcoding system, is to barcode samples instead of spatial regions in tissue. Samples might include different patient samples, multiple samples from the same individual or organisms or samples from different organisms. By barcoding multiple samples with the transposome barcode it is possible to pool all the samples together to perform one single cell sequencing run, and then demultiplex the data and barcodes to determine the identity of each sequence read. For example, the transposome barcoding system can be used to barcode 10 cell lines samples (1,000 cells per sample) and then mix the 10 barcoded cell lines together for a single experiment run on the 10× Genomics single cell RNA sequencing system. Currently highthroughput single cell sequencing systems, such as the 10× Chromium or Mission Bio only allow a single sample to be run on each physical lane of the microfluidics device. Using this sample barcoding system, it is possible to barcode hundreds to thousands of samples, for a single cell sequencing run. The sample barcoding system is flexible and could be used for single cell DNA sequencing, single cell RNA sequencing or single cell Epigenomic profiling. This system will greatly reduce costs associated with all single cell sequencing platforms, through multiplexing, instead of having to run each sample at one time.

### Example 2—Proof-of-Concept

### A. Validation of Transposome Barcoding System with Single Nucleus RNA Sequencing

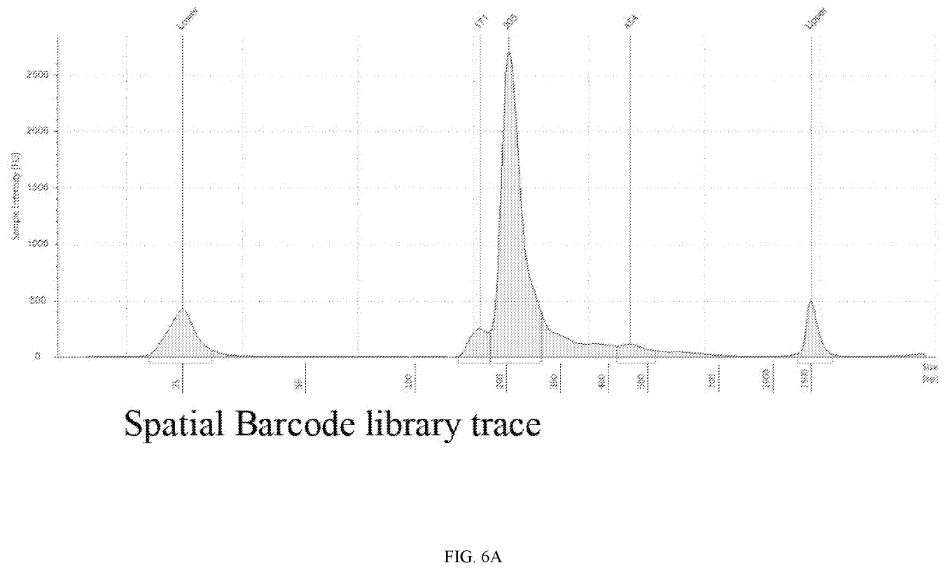

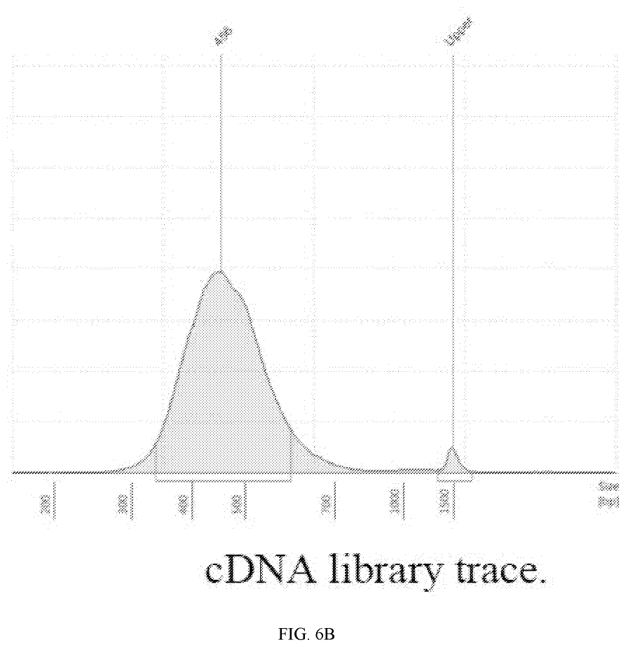

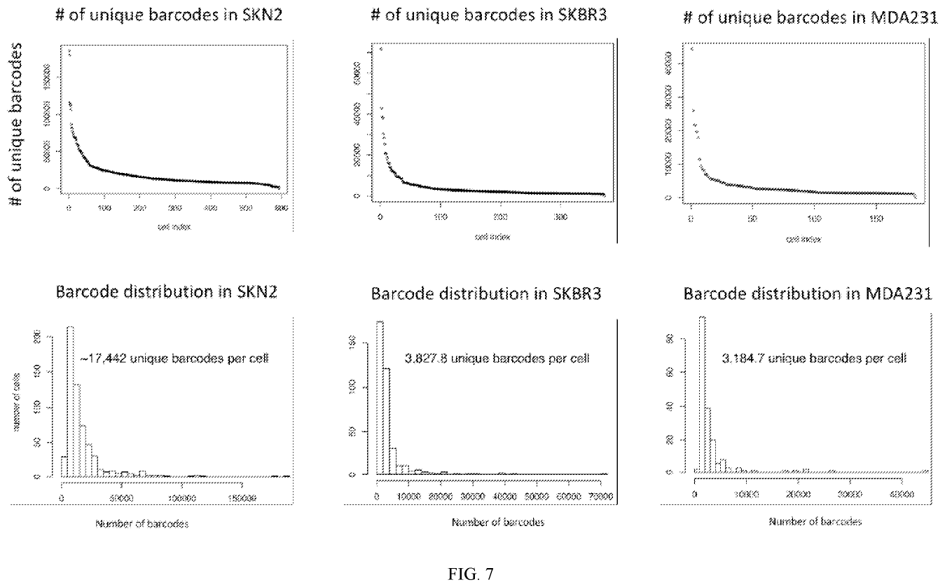

To validate the transposome barcoding system in cell lines, the inventors tested SNUBAR using suspensions of cells first using a single barcode adapter sequences. The inventors tested different transposomes (TDE1) and spatial barcode concentrations (1 uM, 0.1 uM, 0.01 uM) to barcode 30,000 cells in three different cell lines (SKN2, SK-BR-3, MDA-MB-231). After barcoding, the nuclei were washed and mixed equally to prepare one high throughput single nuclei RNA sequencing library (10× genomics Chromium single cell 3′ Reagent kit). After cDNA amplification, the spatial barcode and cDNA libraries were constructed. In FIG. 6, the inventors show the final library trace of barcode library and cDNA library, since the spatial barcode oligos is the same length, there is only 1 peak for all samples. Next-generation sequencing (Illumina, HiSeq4000) of resulted in 175M spatially barcoded reads and 211M cDNA reads. From the sequencing results it was found that 1150 cells (mean 184K reads/cell) were sequenced resulting in the detection of 3409 genes per cell. Clustering and high-dimensional analysis resulted in the single cell RNA profiles separating into 3 groups based on the cell-line of origin (MDA-MB-231, SKN2, SK-BR-3). In this experiment, it was found that 100% of the cells in each cluster were barcoded successfully with the spatial indexes. There were ˜17,442 unique barcodes were detected in SKN2, which were barcoded with 1 uM barcodes oligos, and ˜3,828 and ˜3,185 barcodes were detected in SK-BR-3 (barcoded with 0.1 uM oligos) and MDA-MB-231 (barcoded with 0.01 uM oligos) separately (FIG. 7). These results show that the transposome barcoding system with spatial indexes worked efficiently in solution, with as little as 0.01 uM barcode adapter concentration.

### B. Additional Validation of Cross Contamination in Cell Lines

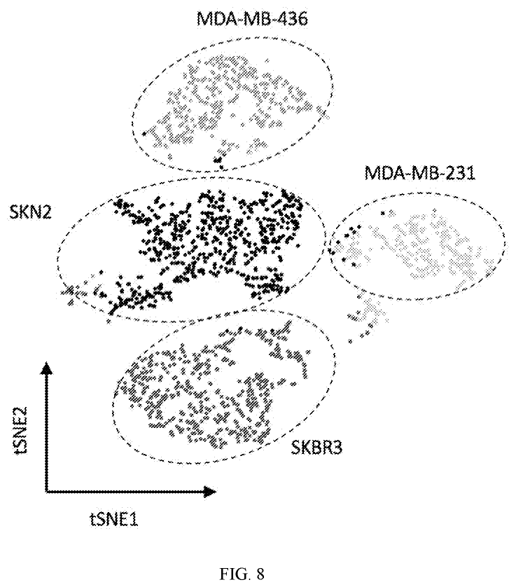

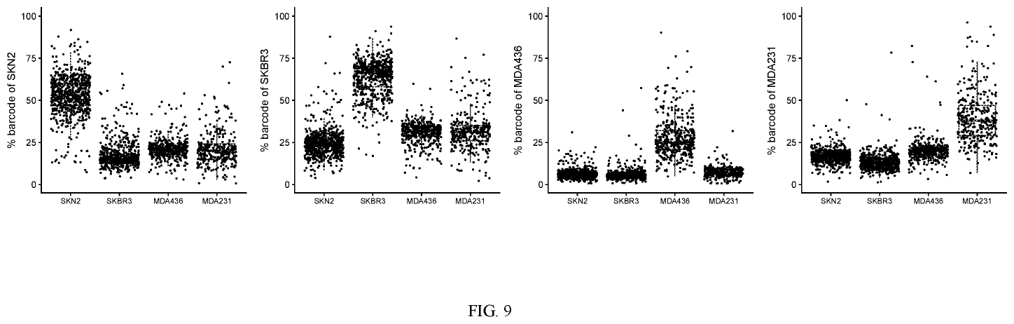

Using the cell lines data, the inventors investigated if the spatial barcodes showed cross-contamination across three cells lines by using different spatial barcodes. This could potentially be an issue if the active transposases are not inactivated when the samples were mixed together. The inventors also investigated whether the spatial barcodes could enter the cell without the transposase to establish the background level of non-integrated barcodes. The inventors used the transposome barcoding system to perform spatial/sample tagging of four different barcodes (two (SpRNA-17-1bc, SpRNA-17-2bc) for tail 1 and 2 (SpRNA-I5-1bc, SpRNA-I5-2bc) for tail 2) with four different cell lines (SKN2, SK-BR-3, MDA-MB-231, MDA-MB-436). After barcoding and washing, the 4 cell lines were mixed to preparing high throughput single cell RNA sequencing libraries for the 10× Genomics system. Next-generation sequencing (Illumina) of 110M barcodes reads and 311M cDNA reads of 2285 cells (mean:136K reads/cell) resulting in the detection of 2909 genes per cell. Based on gene expression profiles, clustering and high-dimensional analysis shows that the cell lines clearly separated into four groups (FIG. 8). In the SKN2 cell line, the barcode SpRNA-17-1bc was most frequent, while in SK-BR-3 the barcode SpRNA-17-2bc was most frequent, and in MDA-MB-231 the barcode SpRNA-I5-1bc was most frequent, and in MDA-MB-436 the barcode SpRNA-I5-2bc was most frequent and could readily be distinguished to infer which cells were barcoded with different spatial indexes (FIG. 9). In summary, these data show that in the presence of Tn5 the barcodes could efficiently enter the nucleus of each cell leading to a dominant barcode in each sample, with minimal background and cross-contamination after mixing the samples together for single cell RNA sequencing.

### C. Validation of SNUBAR for Single Nucleus DNA Sequencing of Cancer Cell Lines

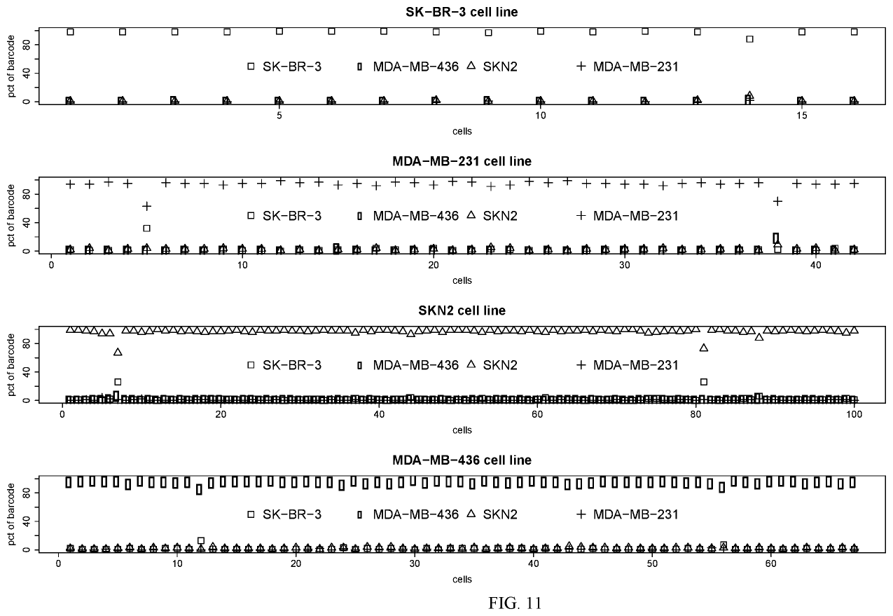

To determine if SNUBAR is compatible with high throughput single cell DNA sequencing methods, the inventors used two different approaches to assemble the transposome barcoding system. In the first approach outlined in FIG. 3A, the inventors hybridized the spatial barcode oligos to the transposome. In the second approach, outlined in FIG. 3B, the inventors used the transposase and spatial barcode oligo with transposase recognition sequences. To test if this method compatible with direct tagmentation based single cell DNA sequencing method, the inventors barcoded four different cell lines (SKN2, SK-BR-3, MDA-MB-231 and MDA-MB-436) with SNUBAR, each barcoded with a different spatial index, and then mixed cells from above four cell lines together to prepare libraries using direct tagmentation chemistry. SNUBAR barcoded single nucleus were flow-sorted into a 384 well plate, and libraries were prepared for each nucleus, then pooled together and sequenced on the Nextseq 500 (Illumina) platform. In final, the inventors got 225 single cells which including 16 SK-BR-3 cells, 42 MDA-MD-231 cells, 100 SKN2 cells, 67 MDA-MD-436 cells. In sequenced SK-BR-3, MDA-MB-231, SKN2, MDA-MD-436 cells, the barcode used to index each cell line are dominate in their specific cell lines respectively (FIG. 11).

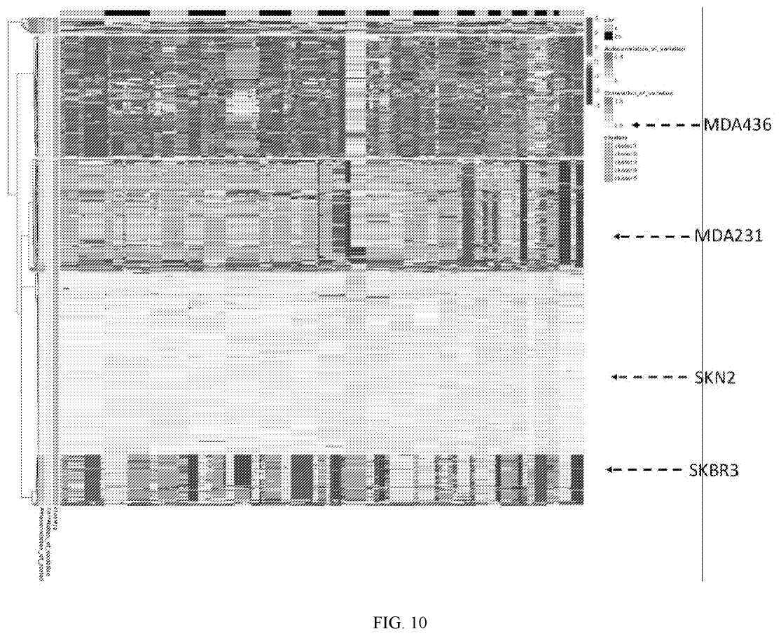

Then to test if SNUBAR compatible with MDA based chemistry, the inventors barcoded 30,000 cells from two different cell lines (SKN2, SK-BR-3) with different spatial barcodes (spDNA-17-4Sbc, spDNA-17-5Sbc) using the first approach and barcoded 30,000 cells from another two cell lines (MDA-MB-231, MDA-MB-436) with two different longer barcodes (SpDNA-v2-9bc, SpDNA-v2-10bc) using the second approach and then mixed them together to prepare high throughput single cell DNA sequencing libraries on the 10× Genomics platform using the CNV reagent kit. To maximize the recovery of the spatial barcodes, the inventors collected the MDA amplified fragments (<100 bp, 100-200 bp and over 200 bp) (Post GEM Incubation in the manufacture instructions), and prepared sequencing libraries. The sequencing data resulted in 80M, 116M and 138M reads from <100 bp, 100-200 bp and >200 bp libraries. In total, 503 cells were sequenced, which includes 190 SKN2 cells, 53 SK-BR-3 cells, 117 MDA-MB-231 cells, 126 MDA-MB-436 cells, and 17 noisy cells that were filtered. Based on the copy number profiles from each cell, the data separate into four distinct clusters with, as expected (FIG. 10). In MDA-MB-436, spatial barcodes were detected in 3.2%, 20% and 79.4% of cells in less than 100 bp, 100-200 bp, over 200 bp libraries respectively. In MDA-MB-231 the spatial barcodes were detected in 2.6%, 12% and 58% of cells in the three different size libraries. However, there were no barcodes detected in another two different libraries of SKN2 and SK-BR-3, which indicates too short barcode fragments can't be amplified efficiently during MDA on the Chromim 10× Genomics system (even if the cells are barcoded efficiently). For MDA-MB-436 and MDA-MB-231, the inventors used longer adapter barcode strategy, which showed much better compatibility with MDA based chemistry, resulting in efficient barcoding.

### D. Application of SNUBAR Barcoding System for Single Nucleus Chromatin Sequencing

To test if the SNUBAR barcoding system is compatible with single nuclei chromatin sequencing methods, such as single cell ATAC-seq, the inventors validated the method in 4 cell lines. SNUBAR was applied to four different cell lines (SKN2, SK-BR-3, MDA-MB-231 and MDA-MB-436) each barcoded with a different spatial index (SpATAC-I5-1bc, SpATAC-I5-2bc, SpATAC-I5-3bc, SpATAC-I5-4bc), and then mixed together to prepare libraries using ATAC-seq chemistry, using a direct-tagmentation based TN5 chromatin accessibility approach after flow-sorting nuclei. SNUBAR barcoded single nucleus were flow-sorted into a 384 well plate, and libraries were prepared for each nucleus, then pooled together and sequenced on the Miseq (Illumina) platform. From these data, the inventors obtained 5M reads, resulting in 8,136 sample barcodes reads in total (2178 for SKN2, 1741 for SK-BR-3, 3071 for MDA-MB-231, and 1146 for MDA-MB-436). These data suggest that if 1M reads were sequenced from each cell, the inventors would obtain approximately ˜2000 barcodes, which more than sufficient to distinguish each spatial barcode from single cells in other samples. In principle, only a single spatial barcode is needed to distinguish each cell from the other spatial barcodes.

Multiplex microdriplet high throughput single cell ATAC seq: In addition to the microplate based single cell ATAC-seq, we have also tested SNuBar for multiplexing droplet-based high-throughput scATAC-seq (eg. 10× Genomics, Drop-Seq). We first prepared nuclear suspensions from two different cell lines (K562 and A20) and performed tagmentation reactions using a transposome with universal tails (similar to Illumina TDE1) for the above two cell lines separately. Two different barcoded oligo adapters were added to the cell lines separately and incubated at 37° C. for another 30 min. Barcoded single nuclei were further loaded into high throughput droplets based single cell ATAC-seq platforms, including the Chromium Single Cell ATAC (Assay for Transposase Accessible Chromatin) Solution (10× genomics) or the SureCell ATAC-Seq Library Prep Kit (Bio-RAD). The ATAC-seq library was prepared following the manufacturer's instructions, and the sample/spatial barcode library was further amplified using primers that hybridize to the universal sequence in the barcodes. The barcoded library and ATAC-seq library are then mixed together and sequenced on the Illumina Nextseq500 platform. From these data we obtained 307M reads, and 8,845 single nuclei from K562 with median fragment 5,475 per nucleus and 8,245 single nuclei from A20 with median fragment 7,680 per nucleus. In K562 single nucleus, the barcode that used to barcode K562 takes around 90% of the total barcodes detected in that single nucleus in average, while in A20 single nucleus, the barcode that used to barcode A20 takes around 70% of the total barcodes, which could clearly distinguish from the background noise.

### Example 3—Sample Barcode Nucleus Delivery Using Oligonucleotides

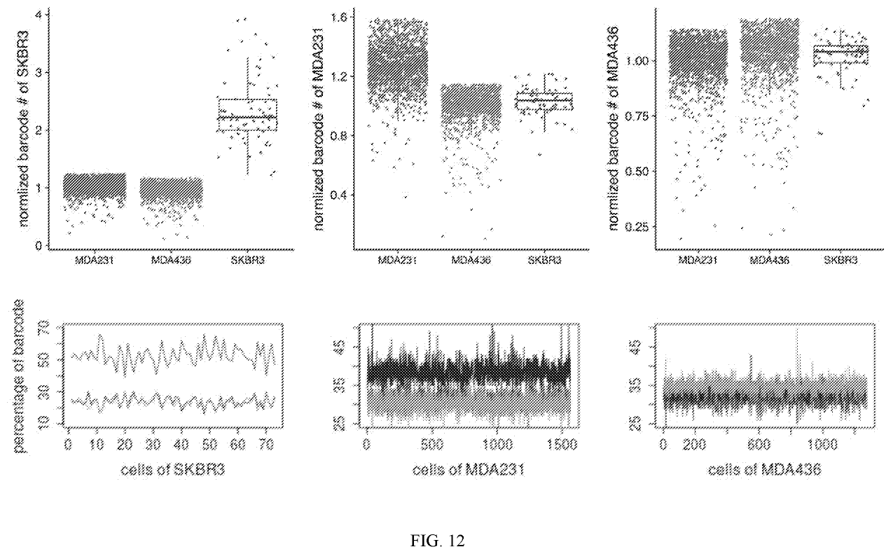

To determine if barcodes could be transferred into single nucleus of cells without the delivery transposase, the inventors performed barcoding on three cancer cell lines (SK-BR-3, MDA-MB-231, MDA-MB-436) using the following protocol. Cultured cells were washed with PBS and lysed with DAPI/NST buffer, then passed through 40 μm filters. The nuclei were washed and resuspended in a buffer, followed by cell counting. Approximately 50,000 nuclei were used to barcode with 1 pmol spatial barcode oligos. For SK-BR-3 and MDA-MB-231, the barcode was incubated at a temperature of 37° C., while for MDA 436, the temperature was 4° C. for 15 minutes. Nuclei then were then washed with resuspension buffer twice. The samples were mixed together to run on the 10× single cell 3′ RNA-seq v2 on the NextSeq500 (Illumina) system. The inventors obtained ˜4500 single nuclei with a median gene count of 2881 genes per cell. The cells were clearly separated into three distinct cluster by SNN and t-SNE according to their gene expression profiles. Next, the inventors determined if the sample barcodes were enriched in the assigned cell lines (FIG. 12, top panel), which was shown in SK-BR-3 and MDA-MB-231, but not MDA-MB-436 (due to the lower incubation temperature at 4C). The same data is displayed as sample-specific barcode percentages in each nucleus (bottom panels), in which the percentages are enriched in SK-BR-3 and MDA-MB-231, but not MDA-MB-436.

### Example 4—Integrating Breast Tissue Architecture and Single Cell Genomics with Spatial Nucleus Barcoding

Single cell RNA sequencing methods are unable to preserve spatial information on cells in their native tissue context. To address this limitation, the inventors developed Spatial Nucleus Barcoding (SNuBar), a method that delivers spatial addresses into nuclei of tissue or cell suspensions prior to single nucleus RNA sequencing. SNuBar was validated using cell line mixture experiments and applied to normal and malignant breast tissues. Analysis of 36 spatial regions in fresh normal breast tissue identified 9 cell types that showed different expression programs that co-localized in three topographic areas (fatty, fibroblast-rich and epithelial). Profiling of 15 spatial regions in a frozen breast tumor identified 4 cell types in the microenvironment and two tumor subpopulations that co-localized with different macrophage expression programs in distinct topographic areas. Our data shows that SNuBar can delineate tissue architecture by integrating macrospatial information with single nucleus transcriptomics in fresh and frozen tissues.

The composition and spatial organization of cell types in tissues are imperative for understanding normal homeostatic functions and the progression of diseases, such as cancer (1). The human breast consists of fatty tissue that supports a ductal-lobular network that is designed to transport milk to nourish offspring (2). In addition to the epithelial bilayer, the breast tissue is composed of adipocytes, fibroblasts, vascular, lymphatic and immune cells (3). Studies using single cell RNA sequencing (scRNA-seq) have begun to delineate the transcriptional programs of breast cell types, but lack knowledge on their spatial organization in tissues, and how this organization influences transcriptional programs and biological functions (4-7). In breast cancer, normal cell types in the microenvironment can undergo transcriptional reprogramming that promote tumor growth. Cell types including carcinoma-associated-fibroblasts (CAFs), tumor infiltrated lymphocytes (TIL), tumor-associated-macrophages (TAMs) and tumor endothelial cells (TECs) have been implicated in promoting tumor progression (8-11). However, there is limited knowledge on how these cell types are spatially organized in tissues and whether this cellular organization can promote invasion, metastasis or resistance to therapy.

Resolving genomic information on cell types in bulk RNA-seq experiments has been challenging, since tissues consists of dozens of cell types and millions of cells. Single cell RNA sequencing methods have emerged as powerful unbiased tools for resolving cell types in normal tissues and the tumor microenvironment, using nano-wells and microdroplet systems (12-17). However, a major limitation is that scRNA-seq methods require the generation of viable cell suspensions by tissue dissociation, during which all spatial information is inherently lost. Some methods that do manage to retain spatial information are limited to measuring small ‘spots’ or spatial regions that consists of many cells. Conversely, several in situ hybridization-based methods may be able to provide single cell spatial resolution, but are limited to measuring targeted genes. Other methods require a priori knowledge of which genes to target and can only image small (<1 mm²) spatial areas.

To address limitations of prior art methods, the inventors developed a transposome-based system called Spatial Nucleus Barcoding (SNuBar) that delivers spatial barcoding into nuclei from a large number of regions for multiplexed single nucleus RNA sequencing (snRNA-seq). The inventors show that this flexible and low-cost method can efficiently introduce nuclear barcodes into a large number of spatial regions that are macro-dissected from a tissue, and enables all of the regions to be pooled together into a single microdroplet experiment. In this study, the inventors validated SNuBar using cell line mixture experiments and applied it to study tissue architecture and transcriptional programs of cell types in normal and malignant breast cancer tissues.

### A. Results

### 1. SNuBar Method Overview

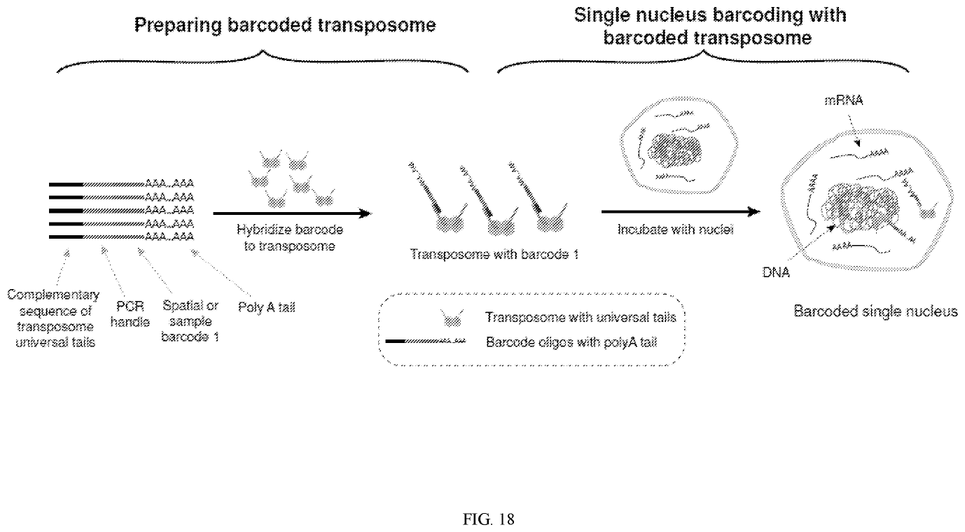

The inventors developed a transposome delivery system that transports spatial barcodes into single nuclei in tissues or nuclear suspensions, after which multiple samples are pooled together for high-throughput snRNA-seq. The delivery system consists of a Tn5 transposome and spatial barcode adapter, the latter consisting of four components: 1) a complementary sequence to the Tn5 transposome universal tails, 2) a PCR amplification handle, 3) a spatial barcode sequence, and 4) a synthetic poly A tail (FIG. 18). To prepare the delivery system, the barcoded transposome is assembled by hybridizing the sample barcodes to the Tn5 transposome, in which one unique transposome is prepared for each spatial region that will be barcoded (Methods). The loaded transposome is then incubated with the tissue or nuclear suspensions where it enters the nuclear membrane and transports the sample barcode adapters into the nuclei.

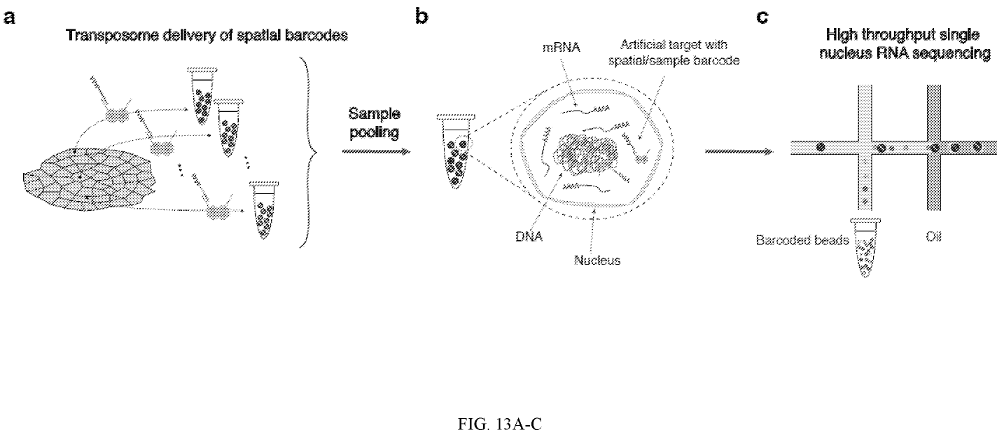

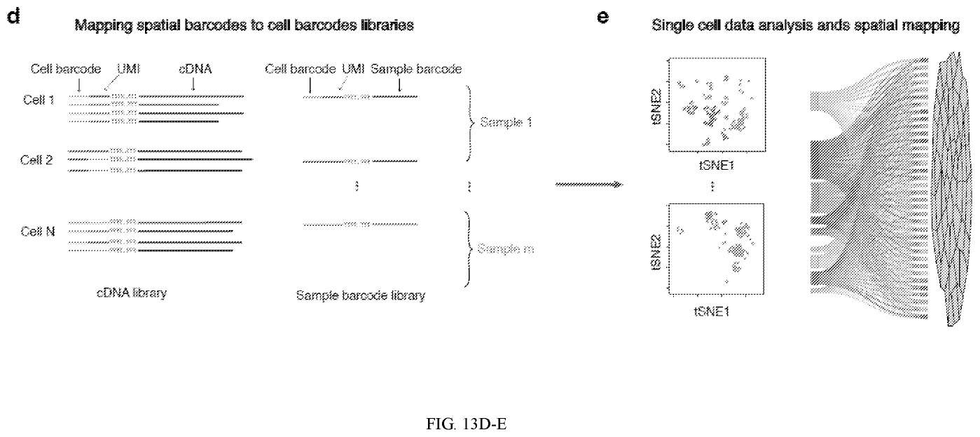

To perform the experiment, fresh or frozen tissue is macro-dissected into many spatial regions (e.g. 10-100) and nuclear suspensions are prepared from each region (FIG. 13A, Methods). The nuclear suspensions from each spatial region is incubated with the loaded Tn5 transposome, containing a different spatial barcode, that is transported across the nuclear membrane. In each nucleus of the barcoded samples, the sample barcode creates an artificial molecular target using the poly-A tail for cell barcode priming and reverse transcription in the downstream microdroplet snRNA-seq experiments (FIG. 13B). After barcoding, the nuclei from all spatial regions are pooled together into a single sample for high-throughput microdroplet snRNA-seq (eg. 10× Genomics, Drop-Seq) (FIG. 13C). Next, the cDNA amplification is performed and two independent sequencing libraries are prepared from 1) the amplified cDNA, and 2) the spatial barcodes. The cDNA and barcode sequencing libraries were then mixed together and sequenced on the Nextseq500 (Illumina) system. From the resulting data, the cell barcode—which is present in both the cDNA and sample barcode reads from each cell—is used to match the expression data to the spatial barcode sequence (FIG. 13D). The final datasets are used to map the expression data of each nucleus to the original spatial location in the tissue (FIG. 13E).

### 2. Cell Line Sample Mixture Experiments

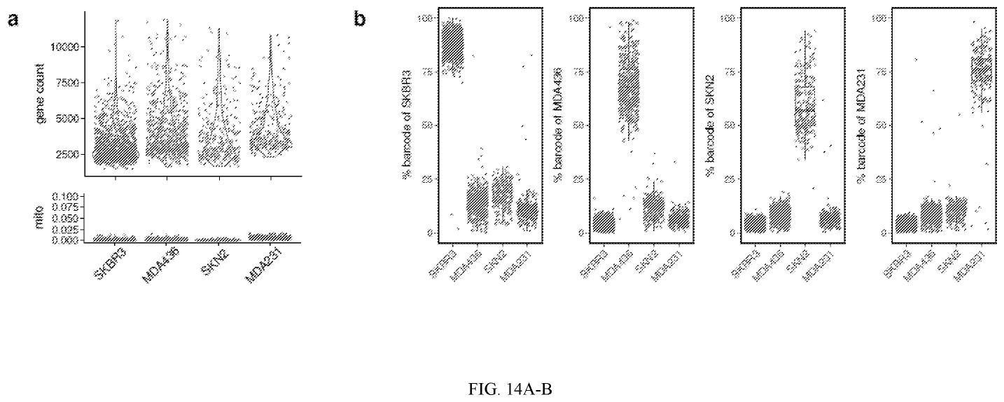

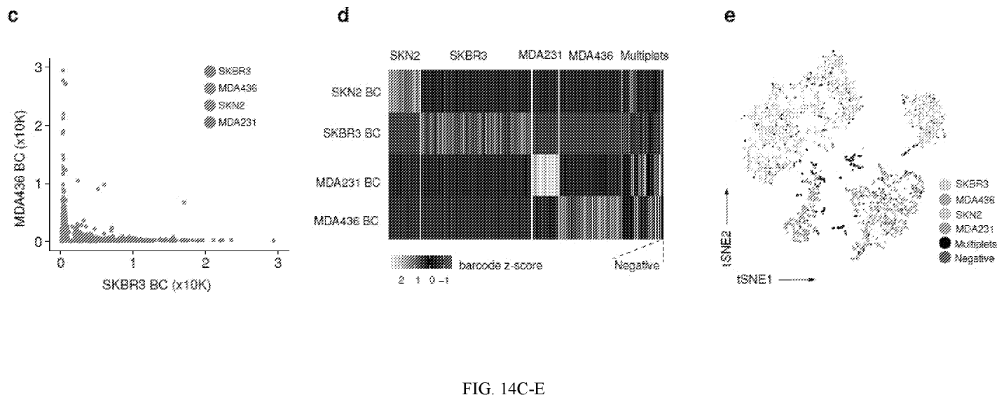

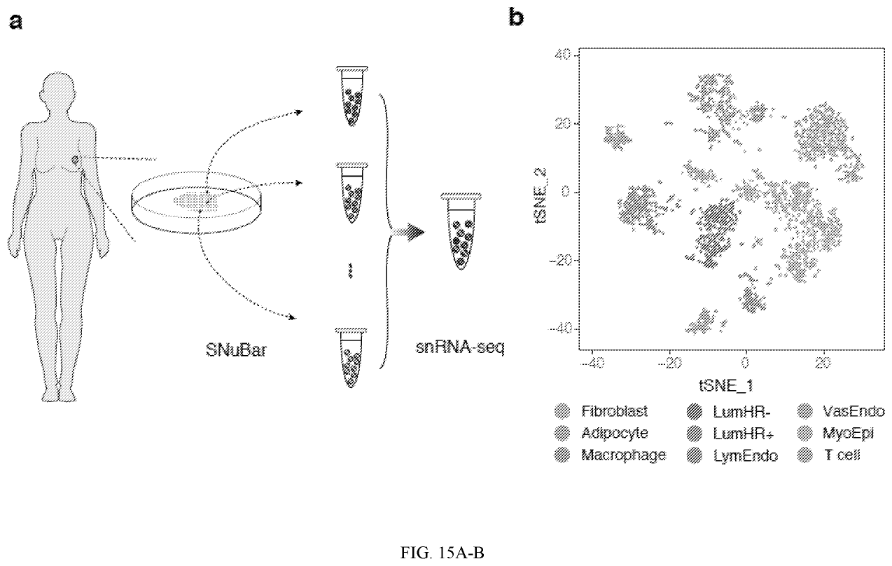

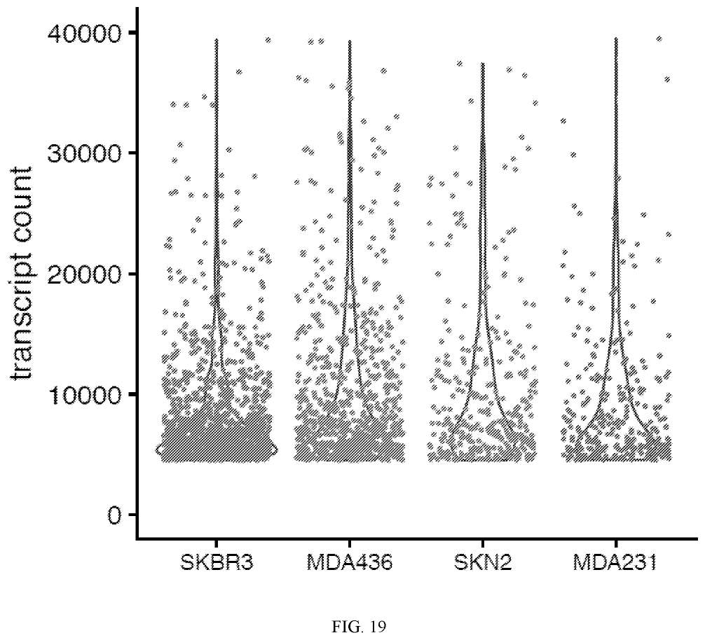

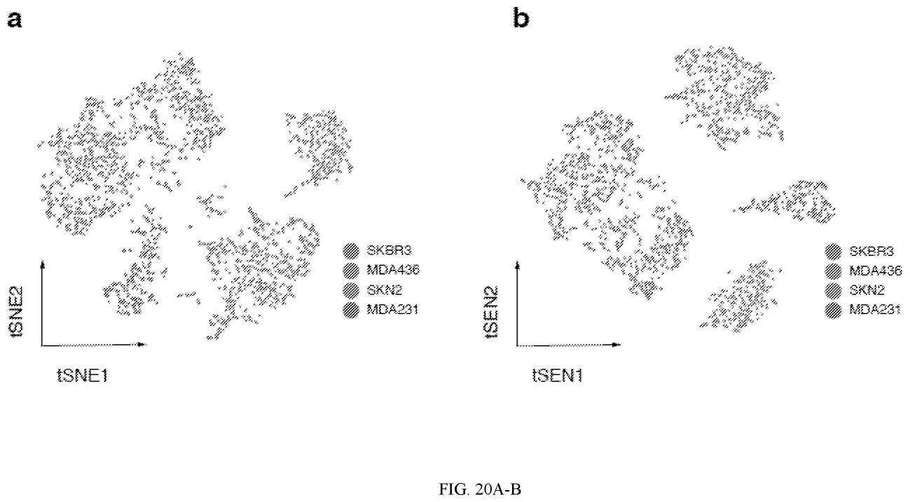

To determine the accuracy and efficiency of SNuBar for multiplexing different samples of nuclear suspensions together, the inventors barcoded four different cell lines (SKN-2, SK-BR-3, MDA-MB-231, MDA-MB-436) with unique spatial/sample barcodes and pooled the nuclei together for high-throughput 3′ snRNA-seq using the 10× Genomics microdroplet platform (Methods). In total, the inventors detected 2,516 nuclei, which resulted in median gene count of 3,170 and unique molecular index (UMI) count of 7,017 per nucleus (FIG. 14A, FIG. 19). The mitochondrial gene percentages in the four different cell lines ranged from 0.1%-0.6% which is about 10-fold lower than a typical scRNA-seq experiment (1-10%) (28), suggesting that contamination from cytoplasm mRNA was minimal (FIG. 14A, bottom panel). High-dimensional analysis identified 4 different expression clusters, which matched to known markers for the cell lines, including SKN-2 (COL1A1, COL1A2, POSTN), SK-BR-3 (ERBB2, KRT7, GRB7), MDA-MB-231 (CD74, KISS1, BIRC3) and MD-MB-436 (PI3, CA9, SAA1) (FIG. 14A, FIGS. 20-21).

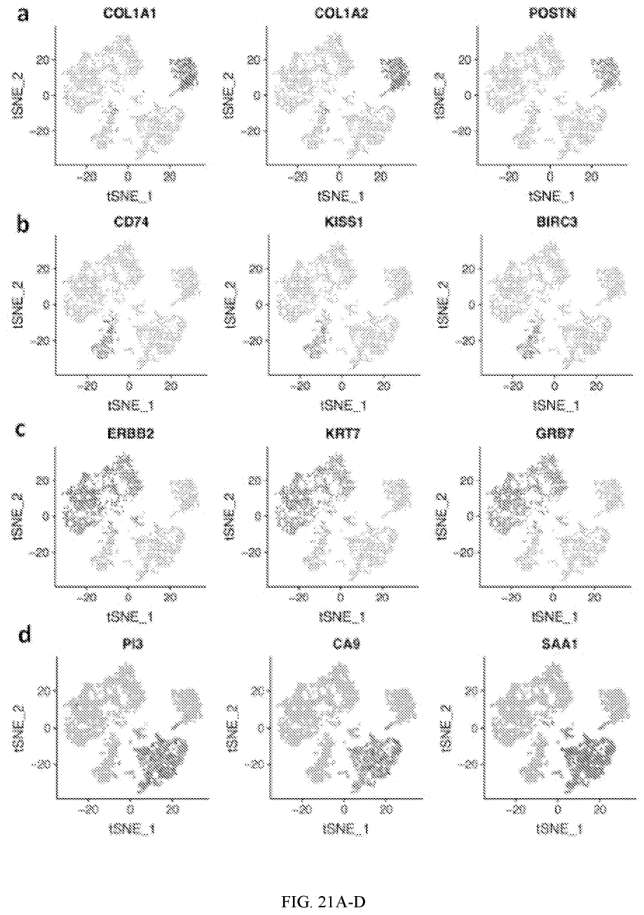

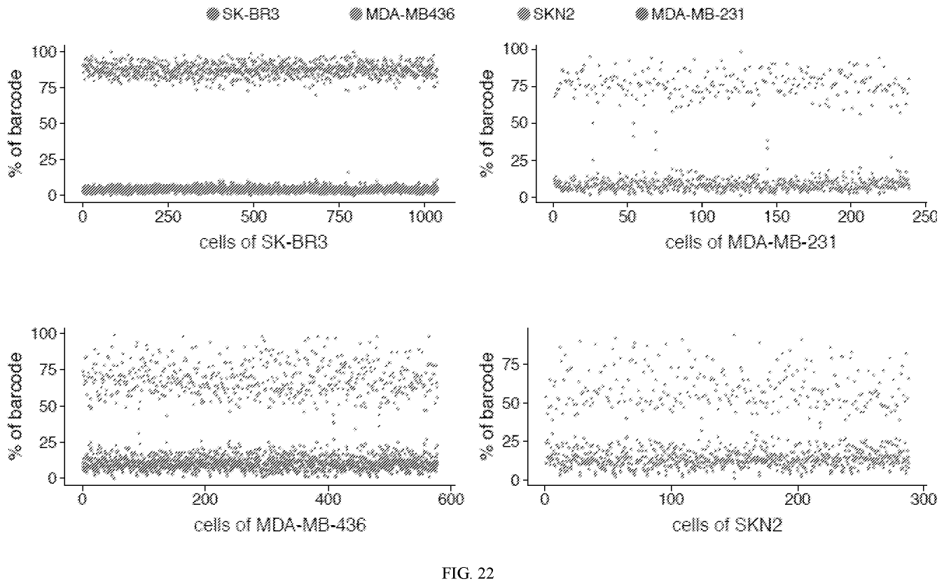

The inventors investigated the per-cell-barcode counts across the four cell lines which showed that the barcodes assigned to each cell line were highly enriched (59.49-87.44%) in the respective sample and were easily distinguished from the background noise (4.44-17.89%) enabling the unambiguous (97.49-99.81%) distinction of most cells (FIG. 14B, FIG. 22).

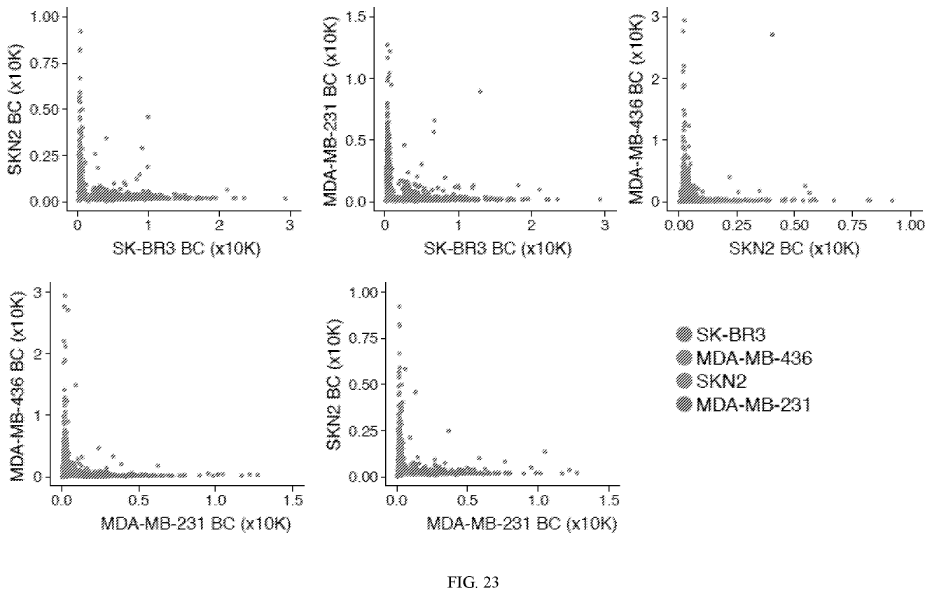

In total SNuBar identified 2,147 singlets (85.33%), 357 multiplets (14.19%) and a small group of 12 nuclei with no barcodes (0.48%) in the datasets (FIG. 14C-E, FIG. 23). The very low percentage of nuclei without barcode assignments, suggests that SNuBar is highly efficient (99.52%) at delivering sample/spatial barcodes into cell line samples. Another unique aspect of SNuBar is the ability to identify and remove cell doublets that cannot be distinguished in standard droplet-based scRNA-seq methods. In microdroplet based approaches the doublet error rate can represent 1-10% of the final dataset and often leads to the false discovery of intermediate cell types (29). By removing the cell doublets from the final datasets, the clustering of the four cell lines was improved substantially (FIG. 14E, FIG. 20B). Collectively, these results show that SNuBar can accurately deliver sample/spatial barcodes into nuclei for multiplexing high-throughput snRNA-seq.

### 3. Spatial Distribution of Cell Types in Human Breast Tissue

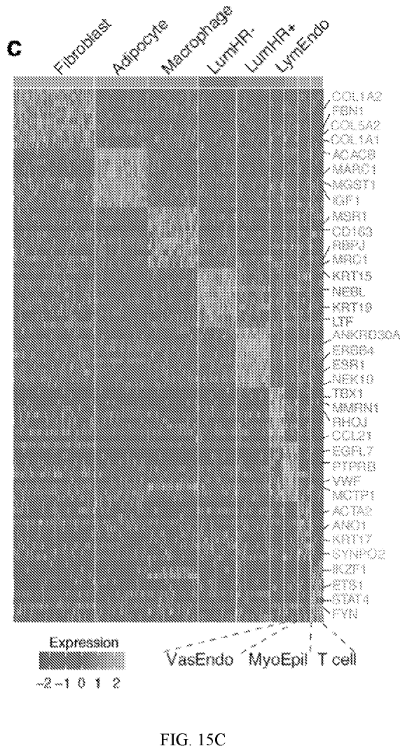

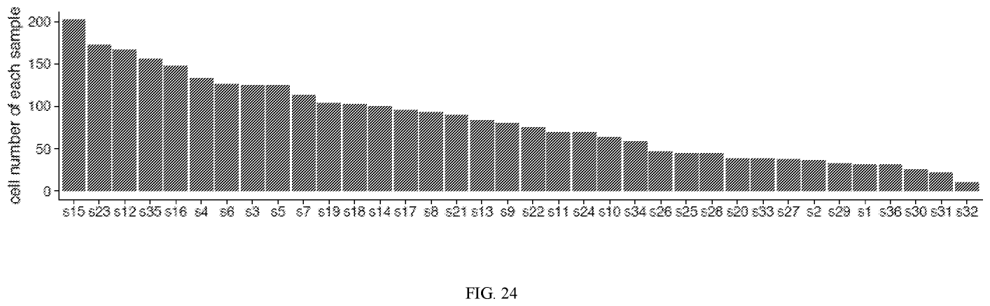

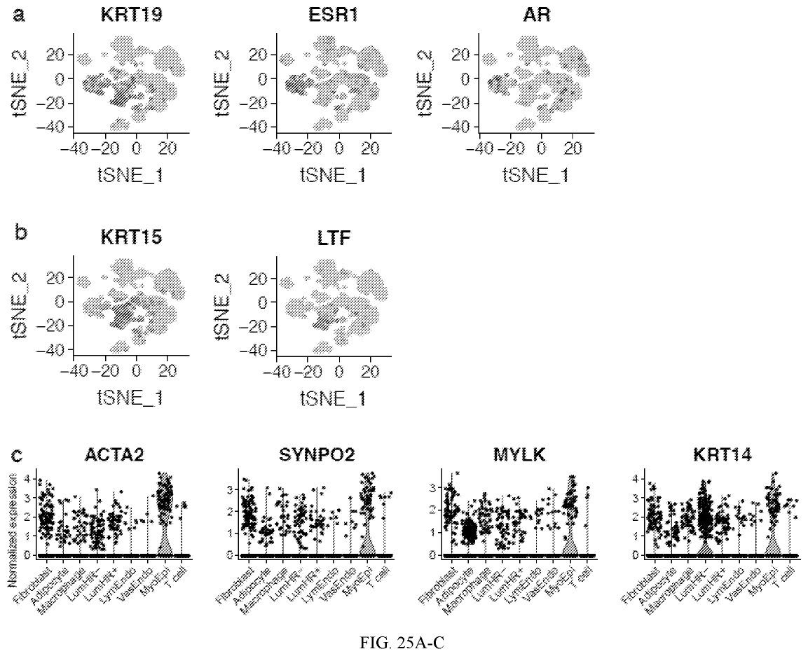

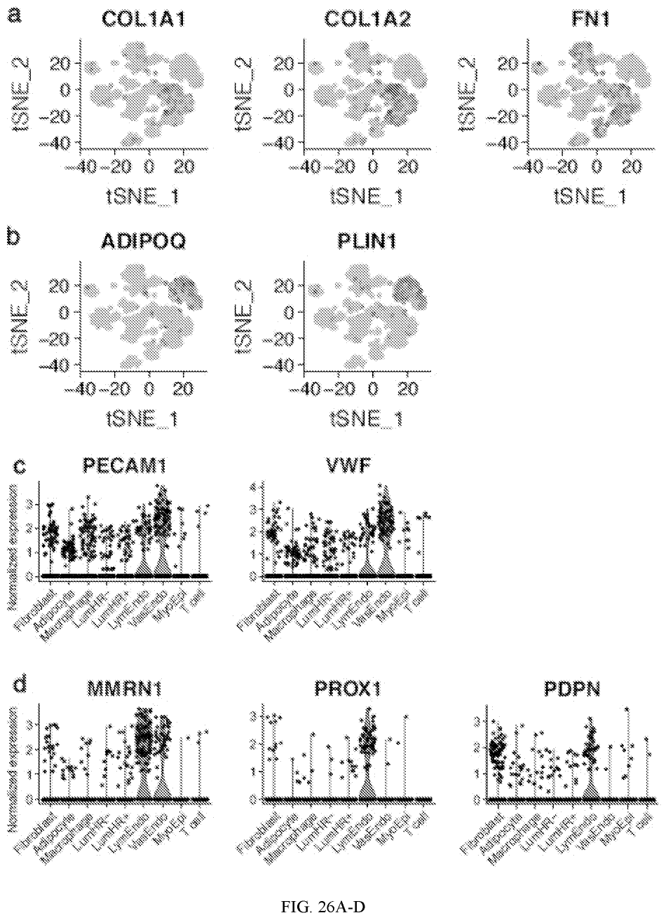

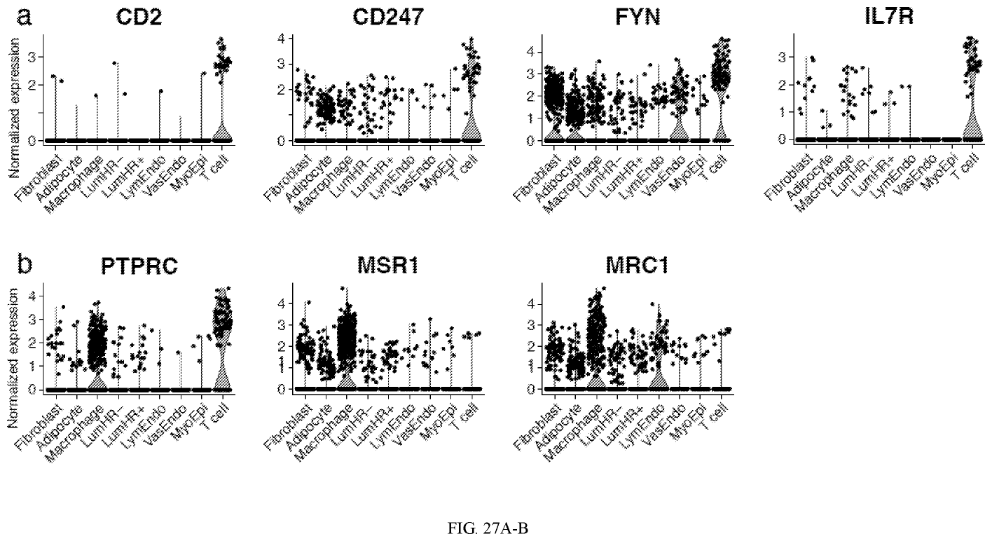

The inventors applied SNuBar to 36 macro-dissected regions from two adjacent fresh tissue pieces collected from a matched normal breast tissue (FIG. 15A, Methods). In total, 2,995 single nuclei were sequenced from 36 regions with an average of 83 cells per sample, after removing doublets and non-barcoded cells (FIG. 24). The nuclei had an average of 1,545 genes and 2,697 UMIs detected per nucleus. To identify cell types, the inventors merged the cells from all spatial regions together for clustering, which identified 9 distinct clusters that corresponded to cell types and known cell types markers (FIG. 15B-C). The major epithelial clusters included hormone responsive luminal epithelial cells (LumHR+: KRT19, ESR1, AR), secretory luminal epithelial cells (LumHR−: KRT15, LTF) and myoepithelial cells (MyoEpi: ACTA2, SYNPO2, MYLK, KRT14) (7, 30), consistent with markers identified in previous studies of normal breast tissues (4, 31) (FIG. 25). The major stromal cell types included fibroblasts (COL1A1, COL1A2, FN1), adipocytes (ADIPOQ, PLIN1 (32)), vascular endothelial cells (VasEndo: PECAM1, VWF (33)) and lymphatic endothelial cells (LymEndo: MMRN1, PROX1, PDPN) (FIG. 26). The major immune cell types included T-cells (CD2, CD247, IL7R (34, 35)) and macrophages (MSR1, MRC1) (FIG. 27). The merged data showed that the fibroblasts were the most abundant cell type (26.92%), followed by adipocytes (17.19%), macrophages (16.38%), and the LumHR−(12.49%) and LumHR+ (10.81%) epithelial cells, while the T-cells, myoepithelial and endothelial cells represented minor (<5%) cell types (FIG. 15B). Notably, an abundant population of adipocytes was detected, which is an elusive cell type that is frequently missed in microdroplet scRNA-seq studies (4, 31) due to the large cell size (>100 microns).

To determine the co-localization of the cell types in the 36 different spatial regions, the inventors performed clustering of cell type frequencies and their corresponding spatial locations (FIG. 15D-E). The data clustered the cell types into three distinct spatial areas (A1-A3), where Area 1 represented a ‘fatty area’ with high frequencies (48%) of adipocytes, while Area 2 was an ‘epithelial area’ that was high in epithelial cell types (55.06%) and Area 3 was a ‘fibroblast-rich’ area with a large proportion of macrophages (39.71%) and fibroblasts (32.24%) (FIG. 15E). The three unbiased clusters of cell types mapped spatially to 3 major topographic areas in the breast tissue (FIG. 15D). This data further revealed the co-localization of adipocytes and fibroblasts in A1, luminal HR+, luminal HR− and basal cells with lymphatic endothelial cells in A2, and macrophages, fibroblasts and vascular endothelial cells in A3 (FIG. 15F). Spatial co-localization of cell expression states in normal breast tissue

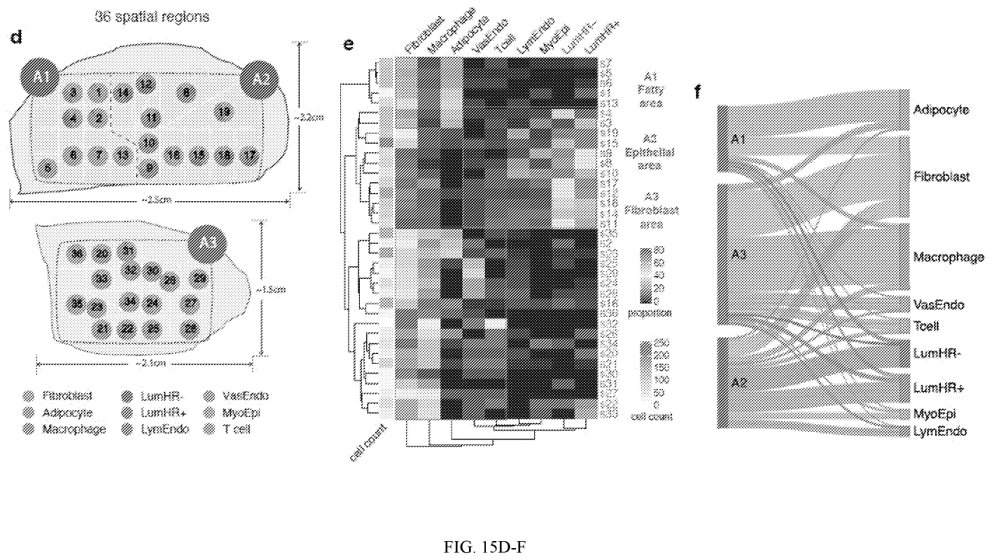

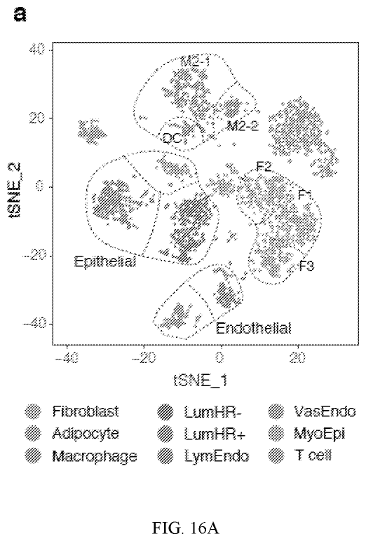

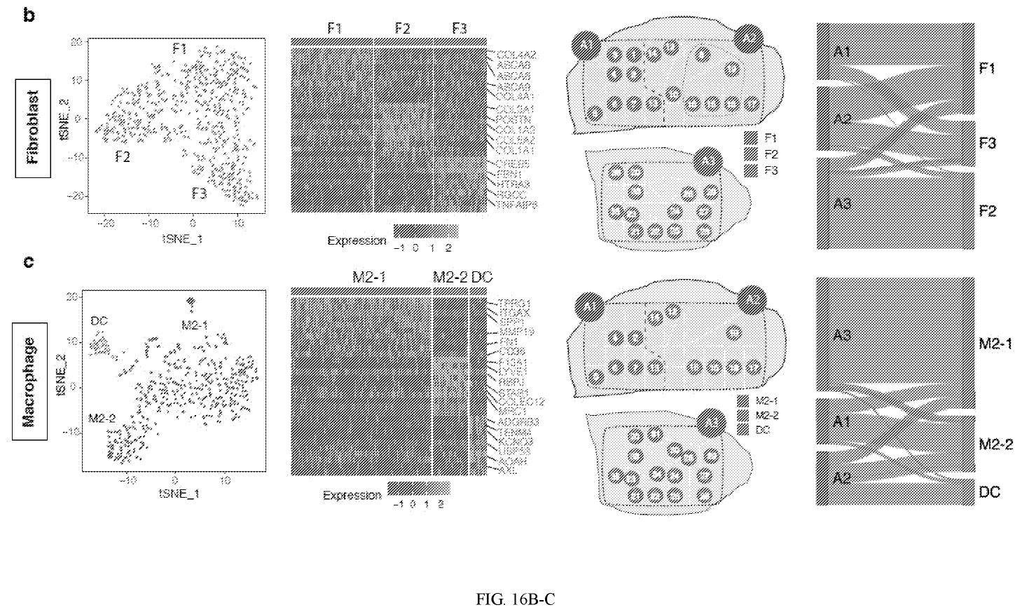

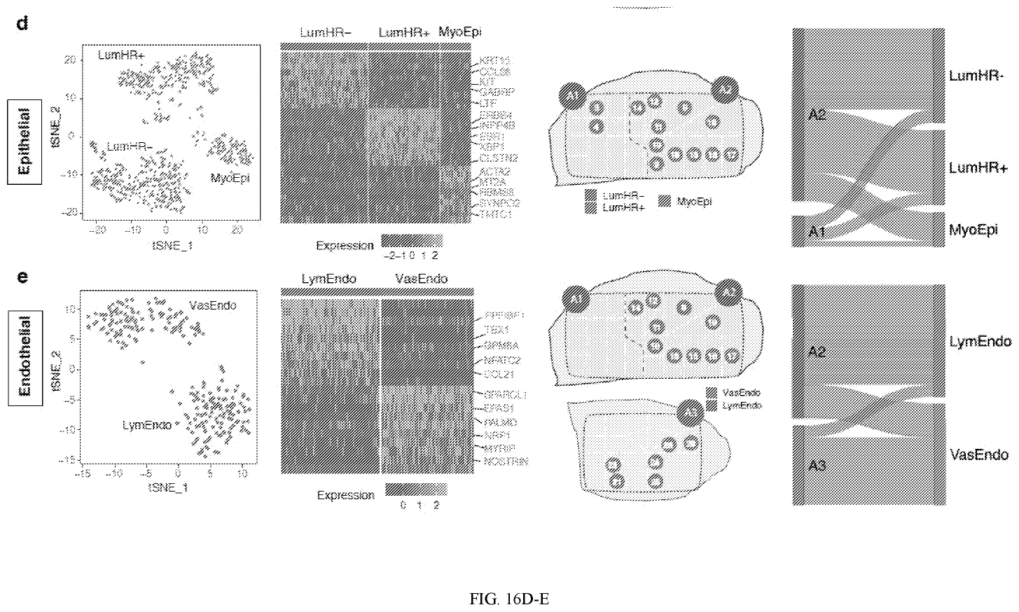

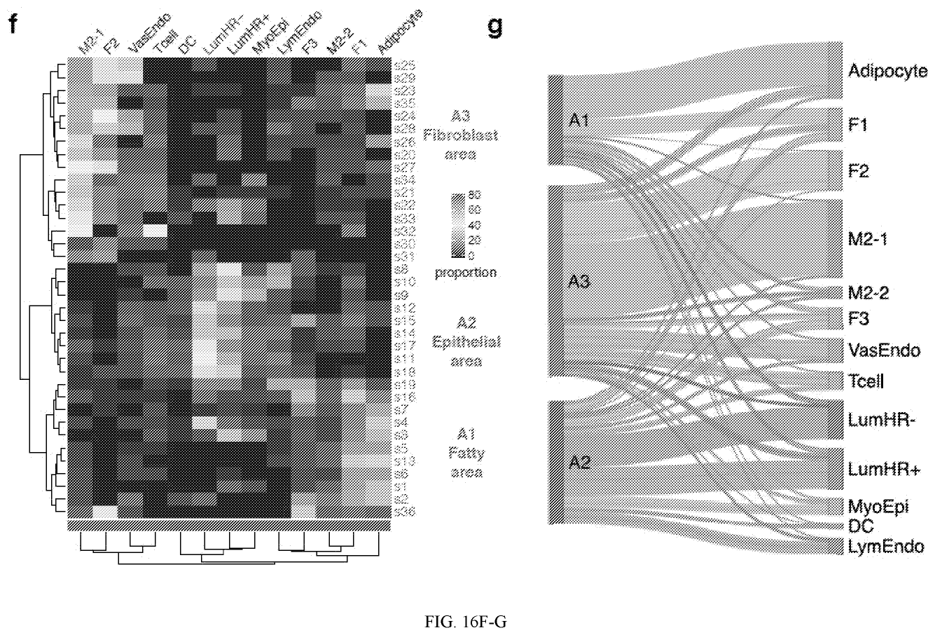

To further investigate differences in the transcriptional programs of the four major cell types (fibroblast, macrophages, epithelial and endothelial) the inventors re-clustered the data from each cell type independently and defined cell expression states across different spatial regions in the breast tissue (FIG. 16). This data revealed multiple expression programs in several cell types, including three fibroblast programs (F1-F3), three myeloid cell states (DC, M2-1, M2-2), three epithelial expression programs (LumHR+, LumHR−, MyoEpi) and two endothelial expression states (VasEndo, LymEndo) (FIG. 16A).

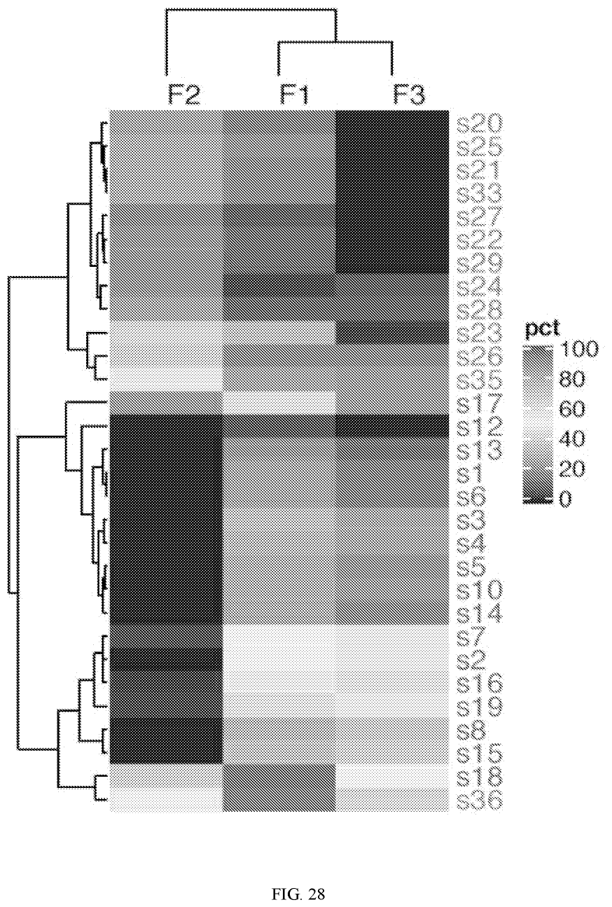

The fibroblast cells showed three distinct (F1-F3) expression programs that corresponded to different spatial areas in the breast tissue (FIG. 16B). The F1 fibroblasts expressed high levels of ABCA transporter efflux proteins (e.g. ABCA6, ABCA8, ABCA9), potentially representing lipofibroblasts, since the ABCA gene family has previously been associated with cholesterol transport (36-38). The F1 fibroblasts were mainly localized to the fatty breast tissue area (A1) and a small part of the epithelial area (A2) (FIG. 16B, right panels). In contrast, the F2 fibroblasts expressed markers associated with activated fibroblasts (FAP, COL1A1, COL1A2, POSTN) (8, 33) and were spatially localized to the A3 area, that also had many macrophages. The F3 fibroblasts expressed high levels of FBN1 and CREB5, and were mainly localized to the A2 epithelial areas (FIG. 16B, FIG. 28).

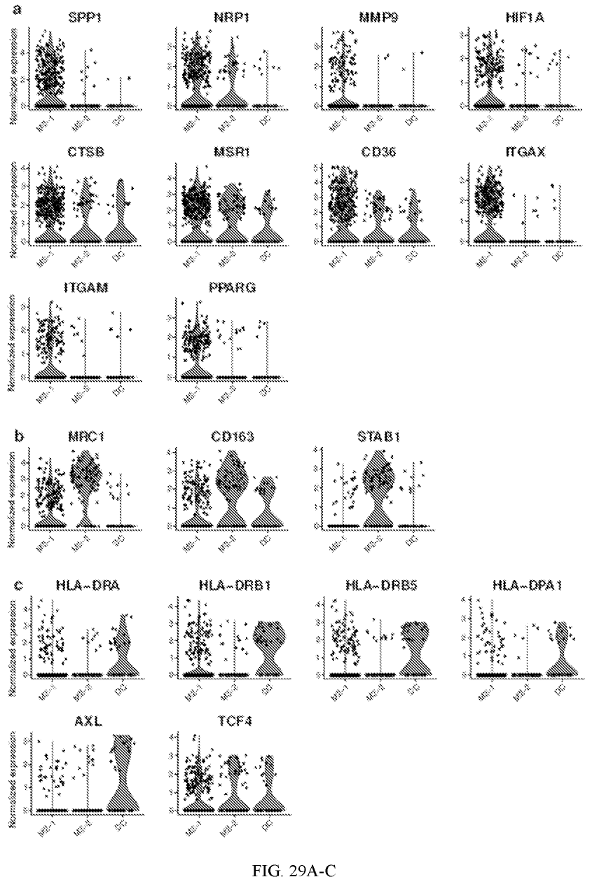

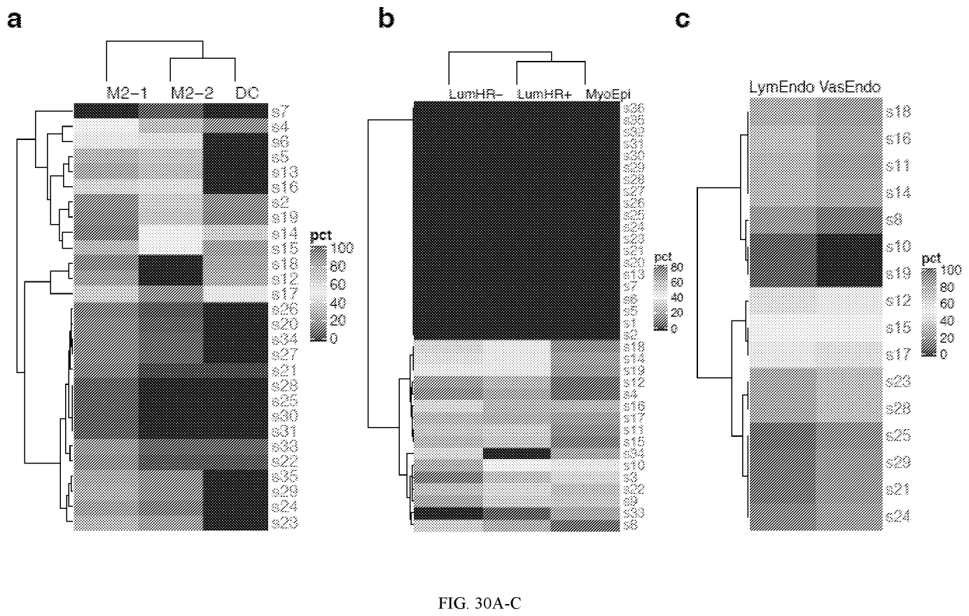

Within the myeloid cell cluster, two sub-clusters of M2 macrophages (M2-1, M2-2) were identified, in addition to the dendritic cell (DC) population (FIG. 16C). The M2-1 macrophages expressed canonical macrophage markers such as CD11B and CD11C, in addition to M2 markers such as MSR1, CD36, PPARG. This cell state was spatially localized to the fibroblast A3 area, where they co-localized with the F2 fibroblasts. Interestingly, the M2-1 macrophages also expressed a number of proangiogenic genes such as MMP9 (39), HIF1A (40), NRP1 (41), CTSB (42), SPP1 (43), ANGPT2 (42) and FGFR1 (44) suggesting that they may be pro-angiogenic macrophages (44, 45) (FIG. 29A). The M2-2 cluster also expressed M2 markers (e.g. MRC1, CD163, STAB1) (46, 47) (FIG. 29B) and were spatially localized to both the A1 (52.86%) and A2 (33.51%) areas (FIG. 30A). The third myeloid cluster represented dendritic cells (DC) and expressed markers such as MHC class II genes, AXL, TCF4 (48) (FIG. 29C) and localized to the epithelial A2 area (FIG. 16C, FIG. 30C).

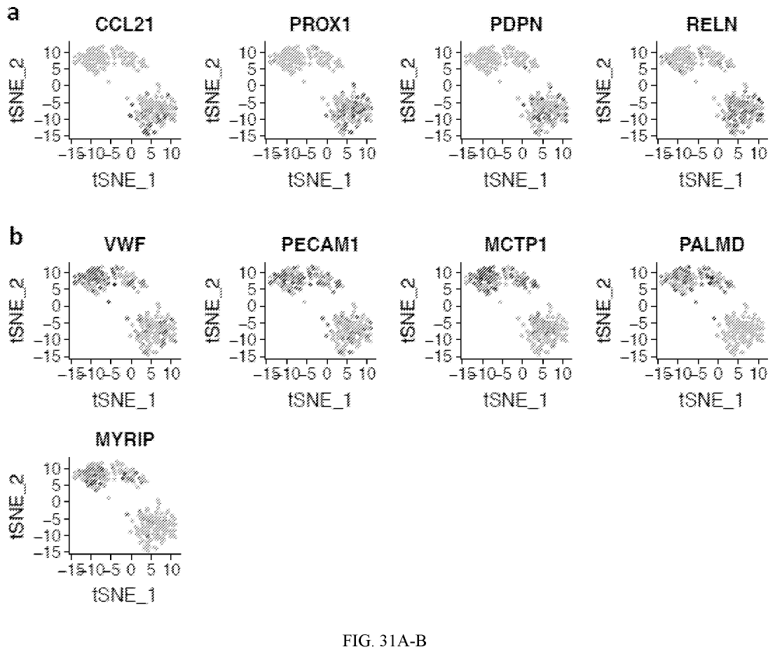

The epithelial cell states corresponded to hormone responsive luminal cells (LumHR+), secretory luminal cells (LumHR−) and myoepithelial cells (MyoEpi) and were spatially localized to A2 (FIG. 16D). Together these cell states comprise the epithelial bi-layer of the ducts and lobules in the human breast (4, 49). Topographically, the three different epithelial cells were co-localized in all of the spatial samples from the A2 area (FIG. 16D, FIG. 30B). The endothelial cell types formed two distinct clusters, that corresponded to distinct cell states: vascular endothelial cells and lymphatic endothelial cells (FIG. 16E, FIG. 31). The VasEndo cells were spatially localized to the macrophage area (A3), while the LymEndo cells were mainly located in the epithelial area (A2). Additionally, no endothelial cells were detected in the fatty (A1) area (FIG. 16E, FIG. 30C). This data was consistent with previous studies showing an association of lymphatic endothelial cells and epithelial cells in the breast by immunofluorescence (50).

To determine the co-localization of different cell expression states in the breast tissue regions, the inventors performed unbiased clustering and spatial mapping (FIG. 16F-G). This analysis independently confirmed our initial assessment, and showed that three major clusters corresponded to the major topographic areas that were defined as the fatty (A1), epithelial (A2) and myeloid (A3) (FIG. 16F). In this analysis, a total of 11 spatial regions clustered together with adipocytes, F1 fibroblasts and M2-2 macrophages that co-localized to the A1 fatty area. Another 9 spatial regions clustered together and corresponded to the A2 epithelial area, including DCs, LymEndo cells, LumHR− cells, LumHR+ cells, MyoEpi cells, F3 fibroblasts, and T cells. The remaining 16 samples clustered together and corresponded to the A3 fibroblast-rich area, which included F2 fibroblasts cells, M2-1 macrophages, VasEndo cells and T-cells. Collectively, these data show that specific cell expression programs co-localized to different topographic areas in the human breast tissue, suggesting that different cell types may have heterotypic interactions that impact their gene expression programs.

### 4. Spatial Expression Programs of Cancer Cells and their Microenvironment

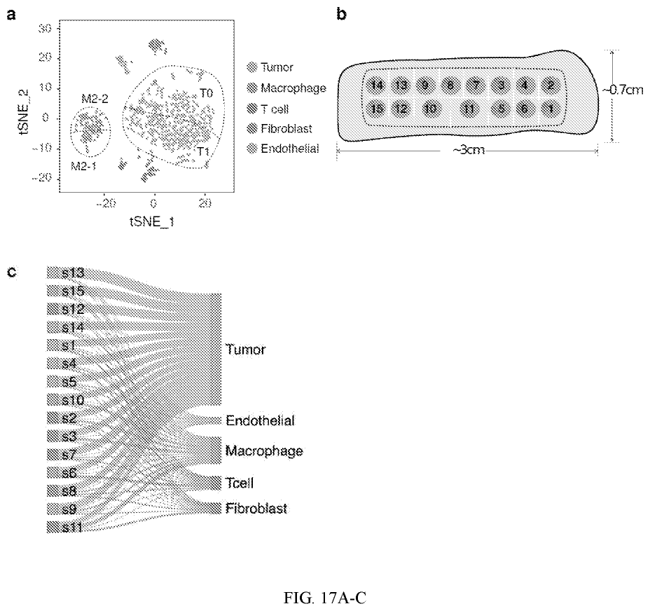

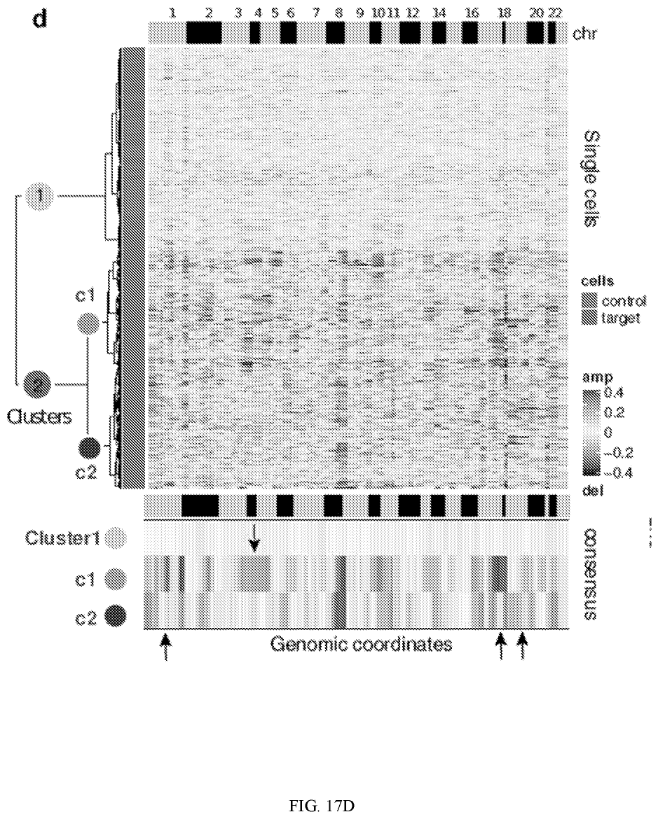

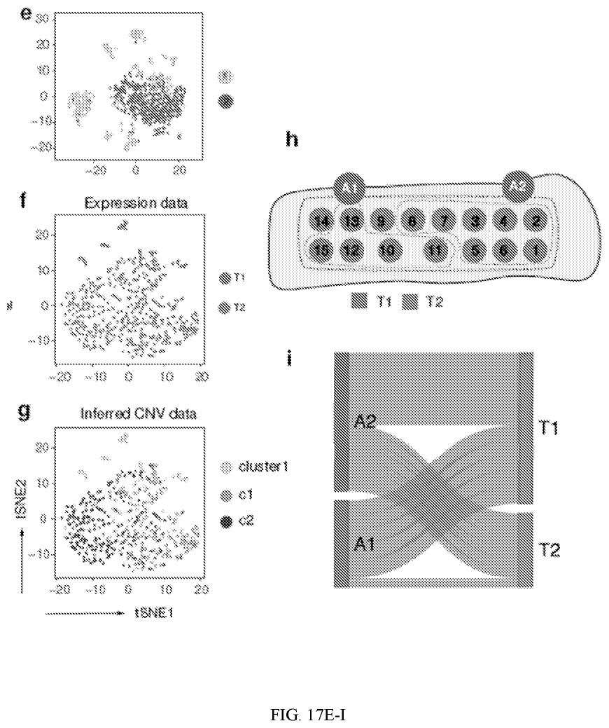

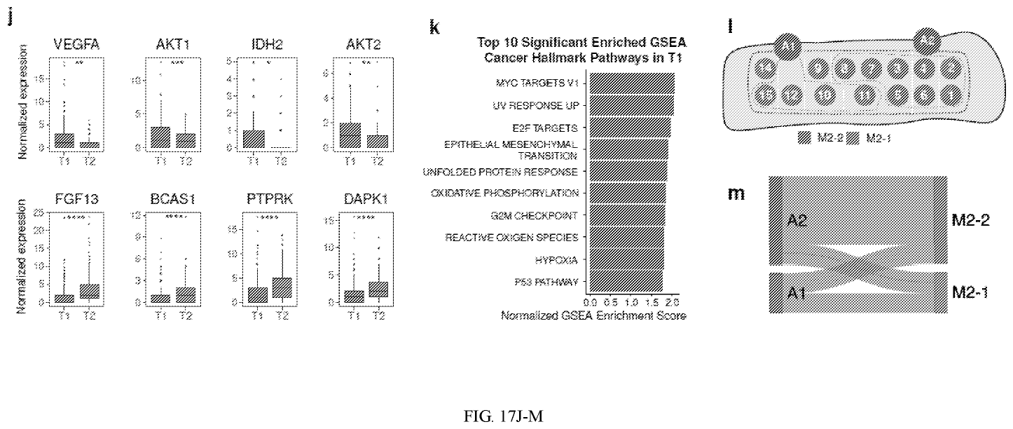

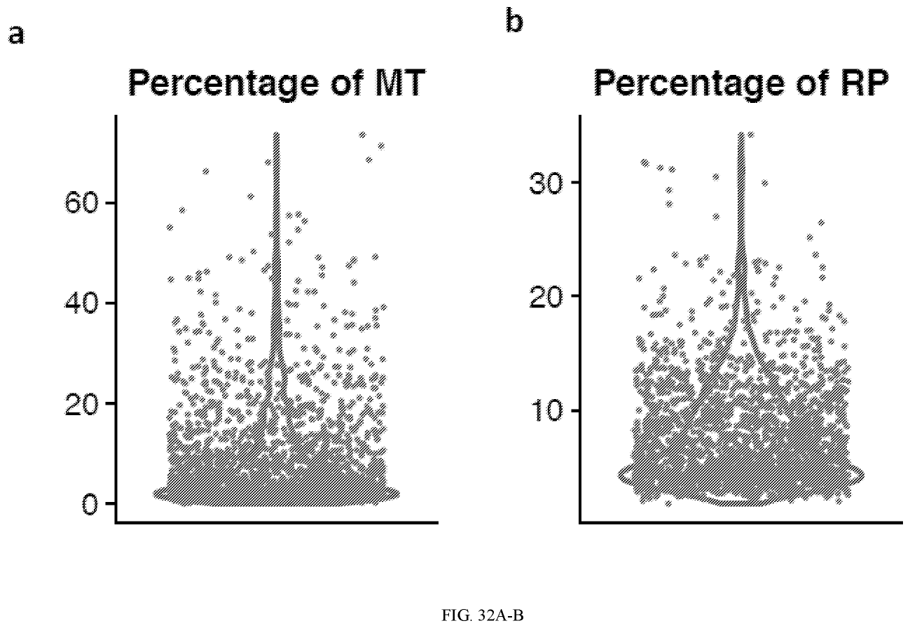

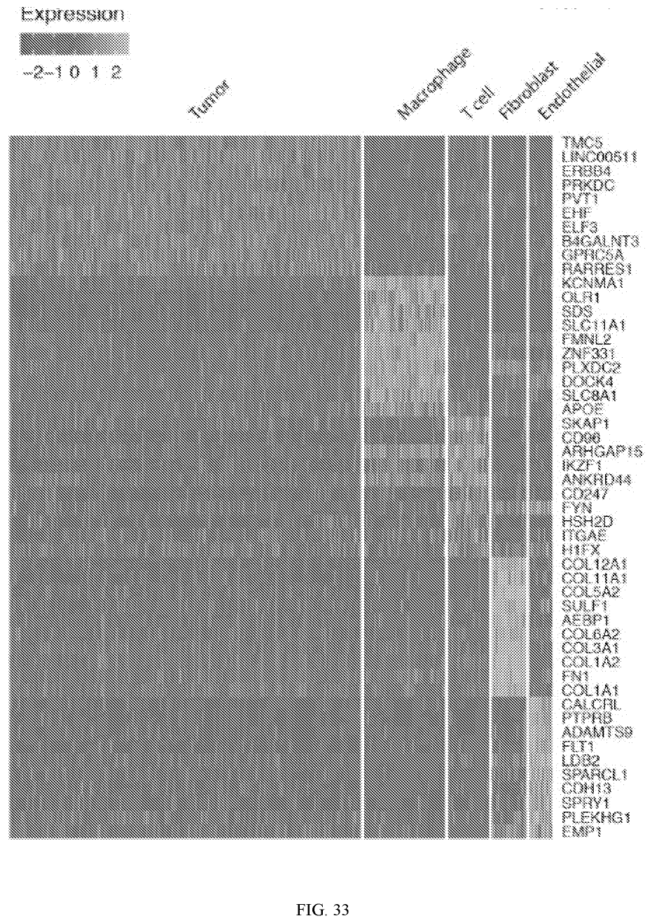

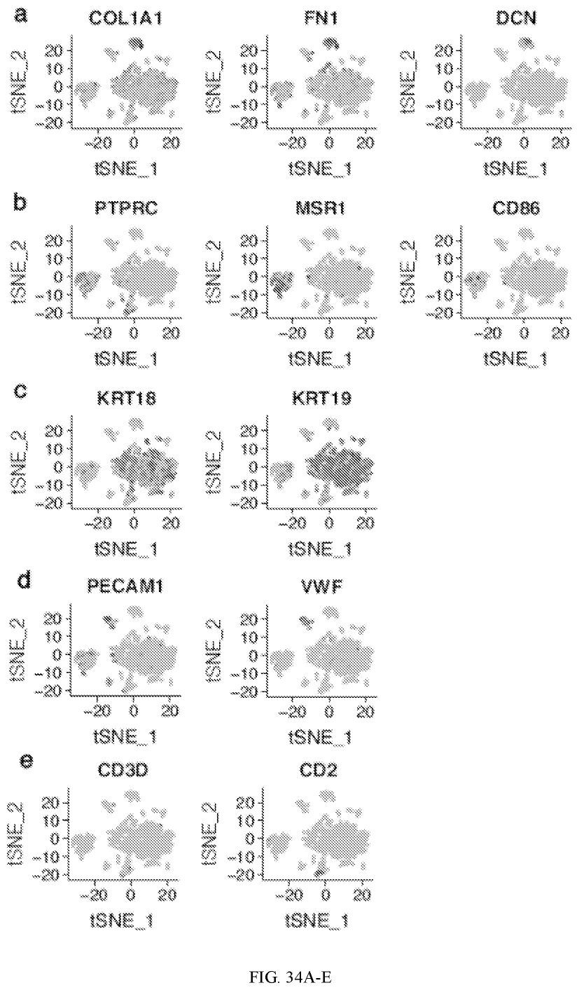

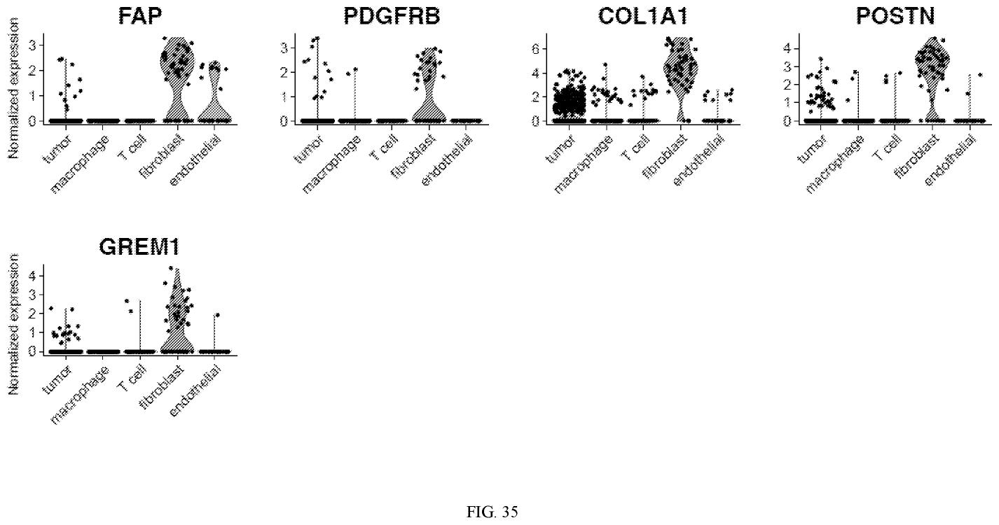

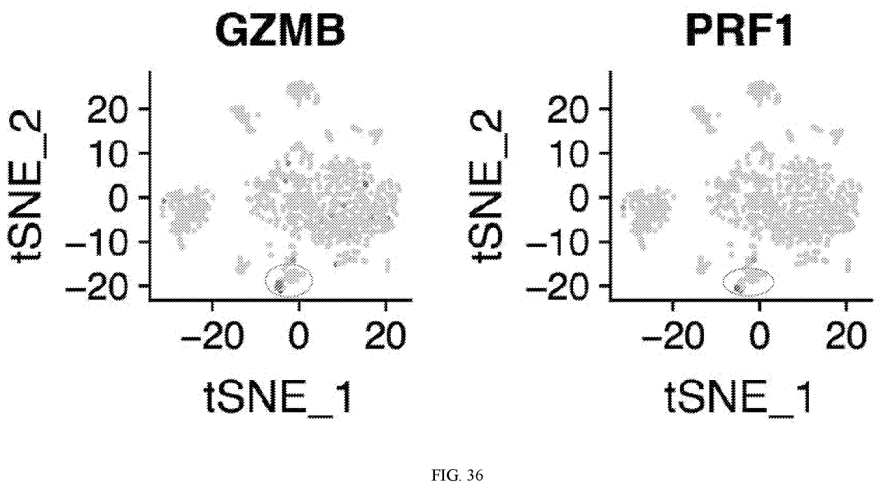

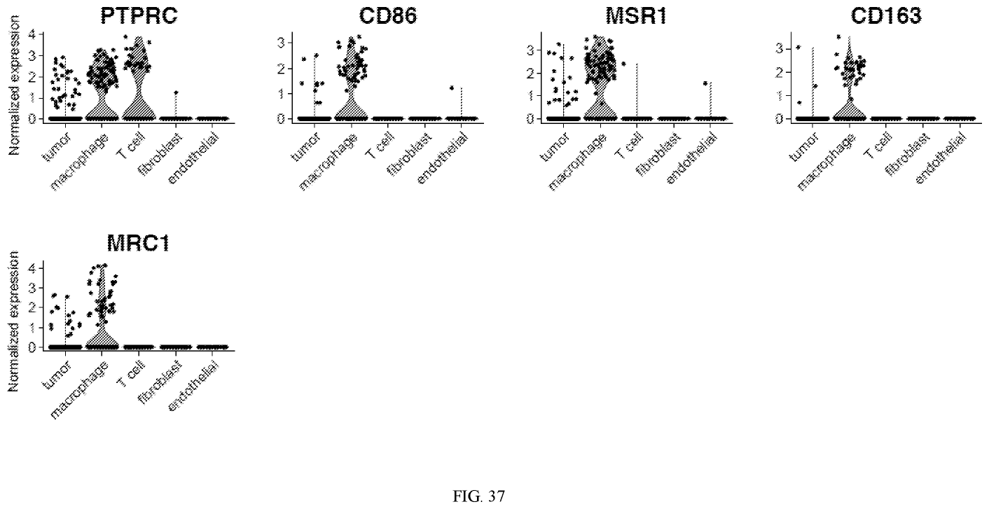

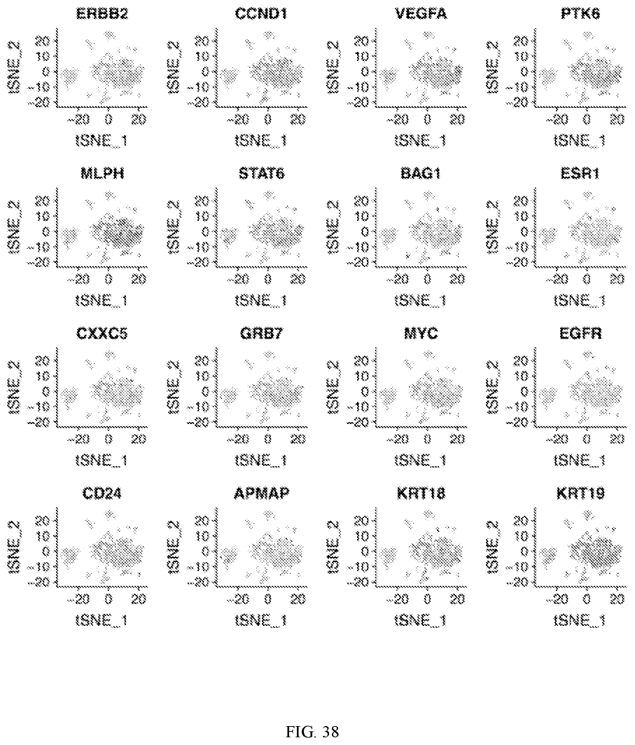

The inventors applied SNuBar to analyze 15 spatial regions that were macro-dissected from a frozen tumor sample from an invasive ER-positive breast cancer patient (ER+, PR−, Her2−) and sequenced 1965 single nuclei (FIG. 17A-B). In comparison to the fresh breast tissue, the frozen sample contained more cells with high percentages of mitochondrial (MT) genes (8.56%±10.26% SEM) and ribosomal protein (RP) genes (7.73%±4.51% SEM), which were filtered from the final dataset (FIG. 32). Four major clusters were identified that corresponded to cell types in the microenvironment, and one cluster represented the tumor cells (FIG. 17A, FIGS. 33-34). Components of the microenvironment included macrophages, T-cells, fibroblasts and endothelial cells. The fibroblast cells showed high expression of normal fibroblasts markers (FN1, DCN) but also showed markers for CAFs including FAP, PDGFRB, POSTN, GREM1, COL1A1 (1, 8, 51) (FIG. 35). The vascular endothelial cells showed high expression of known endothelial markers including PECAM1 and VWF (FIG. 34). The T-cells showed known markers, including CD3D and CD2, and a subset of the T-cells had cytotoxic markers, including GZMB and PRF1 (FIGS. 34, 36). The macrophages expressed CD86 in addition to M2 markers, such as MSR1, CD163 and MRC1 suggesting that they may be tumor-promoting macrophages (FIG. 37).

The tumor cells represented the most frequent cell type (66.53%±12.63%) and were identified in all 15 spatial regions that were profiled. This group expressed epithelial markers including, KRT18, KRT19 and EPCAM, in addition to known breast cancer genes: ERBB2, CCND1, VEGFA, PTK6, MLPH (16, 52, 53) (FIG. 34, 38). To further determine if the epithelial cluster was tumor cells, the inventors calculated genomic copy number aberration (CNA) profiles from the RNA read count data (16) (FIG. 17D, Methods). The inferred CNA data separated the diploid and aneuploid copy number profiles, and showed that most diploid profiles corresponded to expression clusters of cell types in the microenvironment, while the aneuploid profiles corresponded to the epithelial cluster in high dimensional space (FIG. 17E). The inferred CNA data identified aberrations that were shared among all of the aneuploid tumor cells including chromosome 1 p loss, 1 q gain, 8 q gain (MYC) and 18 loss. Moreover, the CNA plots revealed two distinct clusters of aneuploid clones (c1, c2) from which consensus profiles were computed by merging the single cell data (Methods). Comparison of the two tumor clones revealed several copy number differences, including amplifications on 1 q and 17 q, 19, 20 q and deletions of 3 q, 4 and 5p in clone 1, that were not present in clone 2. Similarly, clone 2 had a loss of chromosome 17 q and 19 that were not detected in clone 1.

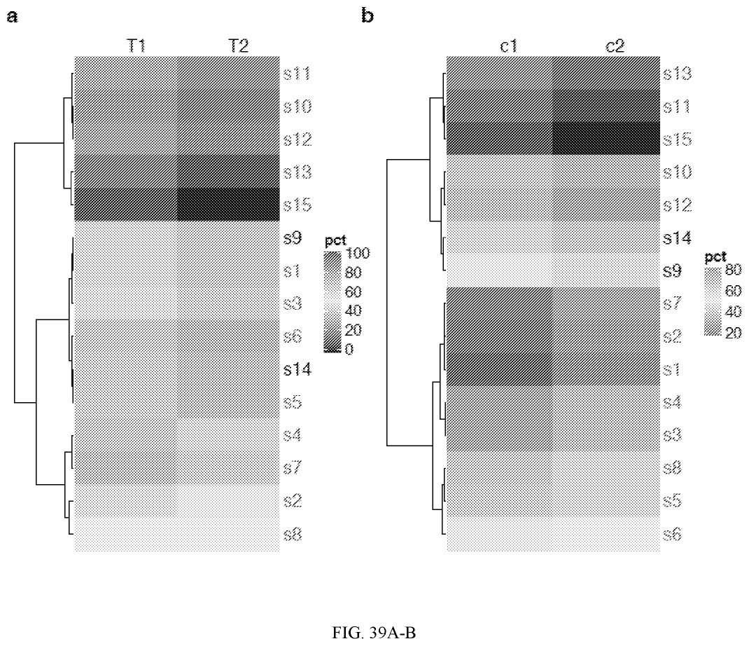

The two CNA clones (c1, c2) occupied different high-dimensional expression space, suggesting that the CNAs may have caused gene dosage effects and divergent expression programs (FIG. 17F-G). The c1 clone was spatially localized to area A1 (regions 10-13 and 15) while clone 2 was more prevalent in area 2 (regions 1-8) (FIG. 17H-I, FIG. 39). The inventors performed differential expression (DE) analysis between the two tumor clones, which identified 534 genes that were significantly upregulated (FDR<0.05) in clone 1 and 224 genes that were upregulated in clone 2. The DE analysis identified several cancer genes, including VEGFA, AKT1, IDH2 and AKT2 that were upregulated in clone 1, and FGF13, BCAS1, PTPRK and DAPK1 that were upregulated in clone 2 (FIG. 17J). To determine whether the expression differences in the two clones impacted their phenotypes, the inventors performed Gene Set Enrichment Analysis (GSEA) analysis using the 50 cancer hallmark signatures (54) (FIG. 17K). The resulting data identified several cancer signatures that were upregulated in clone 1 relative to clone 2, including MYC Targets, Epithelial to Mesenchymal (EMT) transition, Oxidative Phosphorylation (OxPhos), Hypoxia and TP53 signaling (among other signatures), suggesting that clone 1 may have been a more malignant subpopulation in the tumor mass.

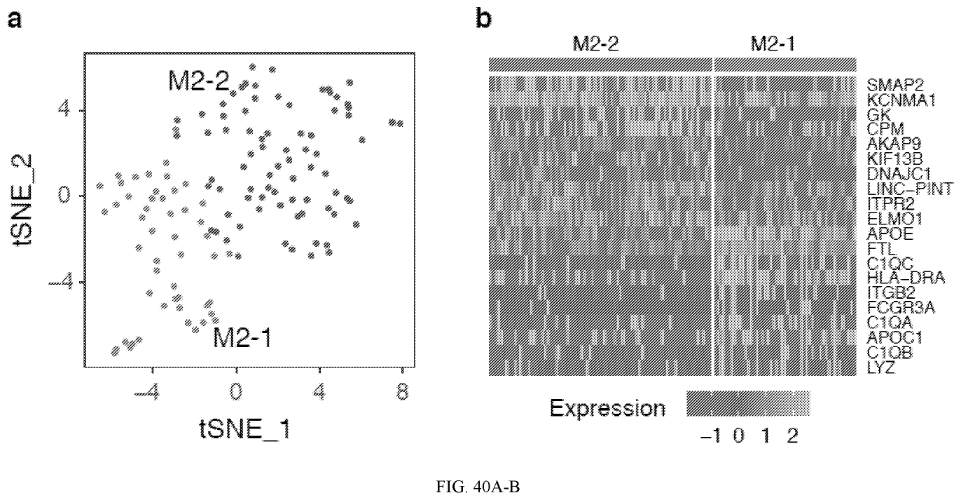

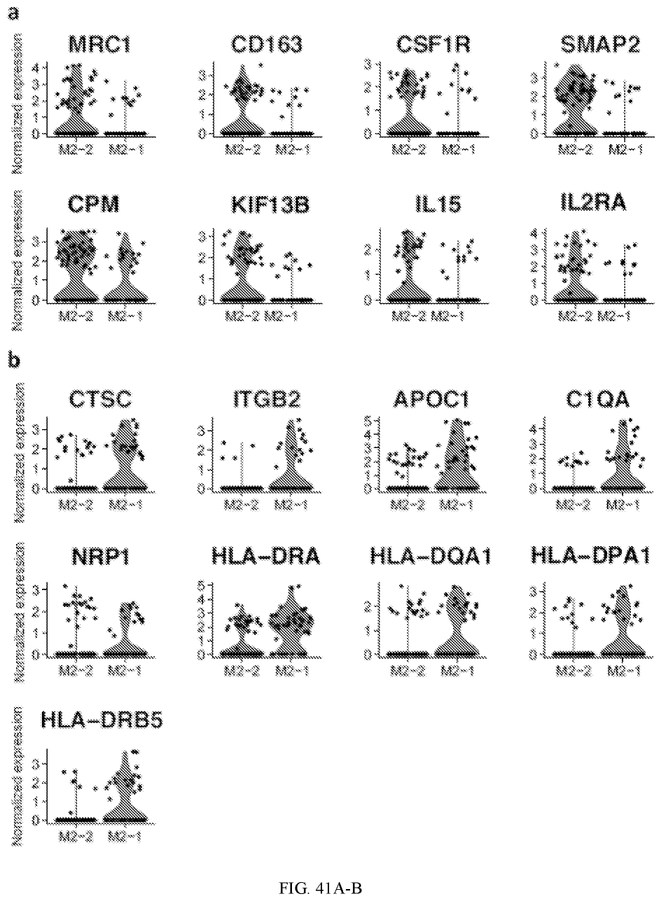

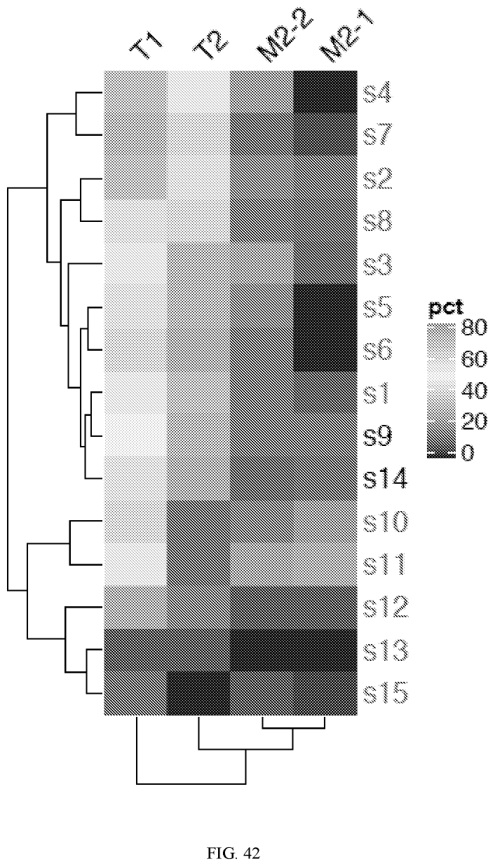

The inventors further investigated the spatial expression of the macrophage cells in the tumor mass, which revealed two distinct M2 clusters: M2-1 and M2-2 (FIG. 40). The M2-2 macrophages showed upregulation of genes including MRC1, CD163, CSF1R, SMAP2, KIF13B, CPM and interleukins IL15, IL2RA (FIG. 41A), while the M2-1 macrophages showed higher expression of CTSC, ITGB2, APOC1, C1QA, NRP1 and MHC class II genes (HLA-DRA, HLA-DQA1, HLA-DPA1, HLA-DRB5) (FIG. 41B). Notably, the M2-2 macrophage corresponded to the same M2-2 cell detected in the normal breast tissue as evidenced by shared markers (e.g. MRC1, CD163). The spatial data further showed that the two macrophages cell states were spatially correlated with the distribution of the different clones. In the A2 area, which contained higher frequencies of the T1 clones, the M2-2 expression state was significantly higher (p=0.01, t-test) than the M2-1 state. In contrast, there was no significant difference between the two macrophages expression states in the A1 area (p=0.45), suggesting that the M2-2 macrophages are associated with the T1 clones. Hierarchical clustering of T1, T2, M2-1 and M2-2 also showed that T2 was colocalized with M2-2 in a spatial context (FIG. 42). These data suggest that the two tumor clones may have had different immune interactions in the tumor microenvironment.

### B. Discussion

Here, the inventors report the development of SNuBar, which, in some embodiments is a spatial barcoding method to label nuclei from macro-dissected tissues prior to performing high-throughput snRNA-seq. Using cell line mixture experiments, the inventors show that SNuBar can efficiently deliver spatial barcodes into single nuclei (>99%) and can multiplexing many samples together for a single snRNA-seq run. Notably, the inventors show that spatial barcodes can be used to distinguish and remove cell doublets from the final single cell datasets. The inventors applied SNuBar to study to study spatial regions from a normal breast tissue sample and an invasive breast tumor sample, which provided new insights into the relationship between spatial topography and the impact of cell type co-localization on expression programs.

In the matched normal breast tissue, the single cell data revealed 9 major cell types that had different expression programs based on their spatial localization to three larger topographic areas (fatty, epithelial or fibroblast-rich). One of the most interesting cell types were the fibroblasts, which displayed three distinct expression programs (F1-F3) across the three topographic areas, that corresponded to different biological functions: lipofibroblasts, activated fibroblasts and epithelial-associated fibroblasts. Similarly, the epithelial cell types, endothelial cell types and macrophages had distinct expression programs that corresponded to the three topographic areas in the breast tissue. This data suggests that cell type expression programs are dictated both by their macro-spatial topographic areas and micro co-localization to local cell type neighborhoods.

In the ER-positive breast tumor, SNuBar revealed the spatial expression programs of tumor cells and 4 different cell types in the microenvironment. In contrast to the normal breast tissue, the microenvironment cell types were uniformly distributed across the 15 spatial regions of the tissue. However, the two tumor cell subpopulations occupied different spatial areas in the tumor mass and one clone (c1) had several increased cancer hallmark signatures (EMT, ROS, oxphos, hypoxia, Myc, TP53 signaling), which suggest it may represent a more malignant clone in the tumor.

SNuBar uses commercially available enzymes (Tn5 transposome, Illumina), has high potential for scalability and does not depend on specific membrane surface for barcoding. Another advantage is that SNuBar can directly barcode single nuclei in frozen tissues (prior to dissociation), since the spatial barcodes enter the intact nuclei directly in the tissue, rather than the plasma membrane, which is often ruptured during freeze-thawing (57).

While SNuBar is limited to measuring nuclear RNA in single cells, this approach has become preferred in the field of single cell genomics for many tissue types (16, 17, 58, 59). Single nuclei RNA-seq can capture larger cell types, complex cell morphologies, provides a truer representation of cell type frequencies in tissues, and allows the analysis of frozen archival tissue samples. To increase spatial resolution of the current implementation of SNuBar, it may be possible to directly apply the oligonucleotide barcodes to micro-regions of tissue sections (prior to dissociation) for snRNA-seq analysis. This application will be an important in future development of the technology, and could potentially increase the spatial resolution to tens or hundreds of cells.

In closing, the inventors show that SNuBar provides a unique approach for spatial barcoding and can provide new insights into the topographic co-localization of cell types and expression states at single cell genomic resolution. Notably, SNuBar is not limited to snRNA sequencing and can potentially be extended to single nucleus DNA sequencing or epigenomic profiling methods (e.g. scATAC-seq) using different adapter sequences. The inventors expected that SNuBar will have broad applications in fields as diverse as cancer research, developmental biology, neuroscience, and immunology, where the integration of single cell genomic information and tissue architecture are key to understanding human diseases.

### C. Methods

### 1. Patient Samples

The frozen tumor and matched normal breast tissues were obtained from the University of Texas M.D. Anderson Cancer Center. The matched normal sample was collected from a DCIS breast cancer patient. The frozen breast tumor sample was classified as ER positive (99%), PR negative (<1%) and Her2 negative with moderate Ki-67 proliferation score and T1a grade 2. This study was approved by the Institutional Review Board (IRB) at the University of Texas M.D. Anderson Cancer Center. Both patients were consented by an informed consent process that was reviewed by the IRB.

### 2. Cell Line Culturing

Cell lines were obtained from the MD Anderson Cell Line Core Facility and tested for mycoplasm contamination and cell line identity by RFLP analysis. SKN-2 was cultured at 37° C. with 5% CO₂ in Dulbecco's Modified Eagle's Medium-high glucose (DMEM, Sigma, D5976) with extra 100 IU Penicillin, 100 μg/mL Streptomycin (Corning™ Penicillin-Streptomycin Solution, Corning™ 30002CI), 2 mM L-Glutamine (Corning™ L-glutamine Solution, Corning™ 25005CI), 1×MEM Nonessential Amino Acids (Corning™ 25-025-CI), and 20% fetal bovine serum (ATLAS, Fetal plus, FP-0500-A). SK-BR-3, and MDA-MB-436 cells were cultured at 37° C. with 5% CO₂ in DMEM (Sigma, D5976) containing 100 IU Penicillin, 100 μg/mL Streptomycin (Corning™ 30002CI), 2 mM L-Glutamine (Corning™ 25005CI) and 10% fetal bovine serum (Sigma, F0926). MDA-MB-231 were cultured at 37° C. with 5% CO₂ in HyClone RPMI 1640 medium without L-glutamine (GE Healthcare, SH30096.01) containing 100 IU Penicillin, 100 μg/mL Streptomycin (Corning™ 30002 CI), 2 mM L-Glutamine (Corning™ 25005CI) and 5% fetal bovine serum (Sigma, F0926).

### 3. Hybridization of the Spatial Barcode Adapters to the Transposome

To assemble the spatial barcoded transposome, the inventors added 1 μl of 1 μM HPLC purified barcode oligonucleotide adapters

(5-′GACGCTGCCGACGACCTTGGCACCCGAGAATTCCA [symbol] 8(A)₃₀-
3′,

the [symbol] sequence represents the 18 bp spatial/sample barcode described in further detail on FIG. 18) to 1 μl TDE1. The reagents are mixed and incubated on ice for 2 h, followed by the addition of 3 μl 1×Tn5 storage buffer (50 mM Tris-HCl, PH 7.5, 100 mM NaCl, 0.1 mM EDTA, 0.1% Triton X-100, 1 mM DTT, and 12.5% glycerol). The mixture is placed on ice for direct use or stored at −20° C. The TDE1 and TD buffer were purchased from Illumina Nextera DNA Library Prep Kit (FC-121-1030), or were purchased separately from Illumina (Catalog #: TDE1: 15027865, TD buffer: 15027866).

### 4. Preparation of Nuclear Suspension from Cell Lines

Cells were washed once in 10 cm Petri dishes with Dulbecco's Phosphate Buffered Saline (Sigma, D8537). To generate nuclei, 5 ml of cold DAPI/NST cell lysis buffer (116.8 mM NaCl, 8 mM Tris base (PH 7.8), 0.8 mM CaCl₂, 38 mM MgCl₂, 400 mg/L BSA, 0.16% Nonidet P-40 substitute (vol/vol, USBiological, N3500), 10 mg/L DAPI) (60) with 0.1 U/μl RNase Inhibitor (NEB, M0314L, 40 U/μl) was added into the plates. Cells were dislodged with cell scrappers, and then transfer into 15 ml tubes. Nuclei suspensions were then passed through 35-40 μm filters (Corning™ Falcon™ Test Tube with Cell Strainer Snap Cap, 352235 or Flowmi® Cell Strainers, BAH136800040-50EA). Cells were centrifuged at 500 g at 4° C. for 5 min, and resuspended with Wash Buffer (1×PBS, 0.04% BSA, 0.2 U/μl RNase Inhibitor), followed by one additional round of washing.

### 5. Preparation of Nuclear Suspension from Fresh and Frozen Tissues

Frozen or fresh tissue was macrodissected into multiple pieces, rinsed in PBS and transferred into 12 well culture plates where the original spatial location of each piece was annotated. The macrodissections were recorded by video camera to ensure that no spatial regions were misplaced. Each dissected piece was minced with no. 11 scalpels in 1 ml of cold DAPI/NST lysis buffer with 0.1 U/μl RNase Inhibitor on ice, and passed through a 36 μm nylon-mesh filter (SEFAR NITEX, 03-36/28, LOT #0474301-00). Nuclei were washed and resuspend in a total of two times.

### 6. Transposome Barcoding of Macrodissected Regions

Approximately 30K-40K nuclei from each cell line or macrodissected tissue piece were incubated with the assembled transposome with the spatial barcode in the following buffer (25 μl 2×TD buffer, 1 μl RNase Inhibitor, 1 μl assembled barcoded Tn5 transposome, 24 μl Wash Buffer with cells). Reactions were incubated at 37° C. for 15-18 min while mixing at 550-850 rpm with 15 s pause and 15 s mixing. The cells were then washed gently with 500 μl Resuspension Buffer (1×PBS, BSA (1%), 0.2 U/μl RNase Inhibitor) or DAPI/NST buffer, followed by incubation on ice for 10-15 min. Nuclei were centrifuged at 500 g for 5 min at 4° C. and the nuclei pellet was resuspended in Resuspension Buffer. Nuclei from different cell lines or tissue pieces were pooled together, filtered and counted using the Countess™ II Automated Cell Counter (Life technologies, AMQAX1000). Nuclei were loaded into 10× Genomics system for single cell RNA 3′ sequencing using the V2 chemistry according to manufacturer's instructions.

### 7. Single Nuclei RNA-Seq Library Preparation

Sequencing libraries were prepared followed by the 10× Genomics single cell RNA 3′ V2 protocol until cDNA amplification step. Then, the inventors spiked 1 μl of a 2.5 μM barcode primer (5′CCTTGGCACCCGAGAATTCCA-3′) into the cDNA amplification reaction mix. cDNA PCR amplification cycles were increased by 1-3 additional cycles over the recommended number, since nuclei have less transcripts compared to whole cells. The amplified cDNA was purified with 0.6×Ampure XP beads. At this ratio, cDNA is bound to the beads and the amplified barcodes remain in the supernatant. Bead-bound cDNA was purified and then used to prepare sequencing libraries according to manufacturer's recommendations. The supernatant containing the barcodes was then purified with additional 1.2×Ampure XP beads (final 1.8×). The sequencing library for the purified barcodes was prepared with the following PCR reaction: 25 μl of 2× KAPA HiFi HotStart ReadyMix, 22 μl purified barcodes and H₂0, 1.5 μl TruSeq RPIX primer (5′-CAAGCAGAAGACGGCATACGAGATNNNNNNGTGACTGGAGTTCCTTGGCACCCG AGAATTCCA-3′) and 1.5 μl TruSeq P5 Adaptor (5′-AATGATACGGCGACCACCGAGATCTACACTCTTTCCCTACACGACGCTCTTCCGA TCT-3′). The PCR was run at 98° C. for 30 s, 4-8 cycles of (98° C. 15 s, 60° C. 30 s, 72° C. 30 s), 72° C. 1 min, and 4° C. hold. The PCR products were further purified with 1.5×Ampure XP beads. cDNA and barcode libraries were then mixed at a ratio of 8:2 and sequenced on the Illumina NextSeq 500 instrument using the following read lengths: Read1: 26 bp, Read2: 58 bp, Index read (17): 8 bp.

### 8. Data Pre-Processing

The 10× Genomics CellRanger (v2.2.0) mkfastq was used to demultiplex libraries by sample indices and convert the barcode and expression data to FASTQ files. The FASTQ files of expression libraries were further processed using the 10X CellRanger count pipeline. Reads were aligned to the human GRCh38 premrna reference (v1.2.0). The gene matrix output by CellRanger was normalized and analyzed with the Seurat R package (v2.3.4) (61). Single nuclei with low numbers of genes (N<200) were filtered from the final dataset. FASTQ files of the spatial barcode library were converted into a sample barcode matrix using CITE-seq-Count (63), using the following arguments: -cbf 1 -cbl 16 -umif 17 -umil 26 -hd 2, and using the cells called by CellRanger as white list.

### 9. Cell Line Data Analysis

For the cell line mixture experiment, the inventors filtered the nuclei with gene counts (N>12,000), and nuclei with a mitochondrial gene percentage higher than 0.02. Sample barcodes were demultiplexed using the Seurat built-in ‘HTOdemux’ function using the sample barcode matrix generated by CITE-seq-Count, with a cutoff above the positive quantile of 0.99. Detection of multiplets and negative cells were removed from the final datasets, and singlet data was further subjected to log normalization with scale factor (N=10,000), and further scaled by UMI count and mitochondrial percentages. The scaled data was further subjected to PCA followed by non-linear dimensional reduction (t-SNE). Wilcoxon rank sum test were performed to identify feature genes of each cluster.

### 10. Tissue Data Analysis

For fresh and frozen human breast tissues, the inventors used the deMULTIplex R package⁵⁶ instead of Seurat HTOdemux function to demultiplex the spatial/sample barcodes, since HTOdemux cannot handle a large number of sample barcodes. Detected multiplets with multiple barcodes and negative cells with no assigned barcodes were removed from the final dataset, and singlet data was further imported into the Seurat R package. Single nuclei with high gene counts (N>9,000) and a high mitochondrial gene percentage (M>4%) were further filtered. For the frozen tissue sample, the cells with ribosomal proteins over 10% were also was filtered from the final datasets. The filtered singlet data was further used to perform log normalization with a scale factor (S=10,000), and further scaled by UMI counts and mitochondrial percentages. Scaled data were used for PCA and t-SNE for high-dimensional analysis. Wilcoxon rank sum test or DEseq2 (63) methods were performed to identify differentially expressed genes.

### 11. Copy Number Inference from Single Cell RNA Data

To infer copy number aberration (CNA) from single nuclei RNA-seq data, the inventors used our lab previously published method (16) that calculated CNA from log transformed gene matrix using a “moving average” approach. In brief, expression was quantified as log(count+1), and all genes with average expression across all cells<0.3 were removed. Relative expression of each cell was calculated by removing the average expression of normal cells, and was further corrected to 2 or −2 if the values were larger or lower than 2. Copy number value of each gene was defined as the sliding average value with a window size of 50 and centered at each gene.

### 12. Gene Signature and Pathway Analysis

To perform gene signature and pathway enrichment analysis, the inventors first used DESeq2 (63) (v1.22.2) to perform DE analysis of the two different tumor subpopulations, using the following arguments: test=“LRT”, sfType=“poscounts”, reduced=˜1, useT=T, minReplicatesForReplace=Inf, minmu=1 e-6, fitType=‘local’, and further shrunken with lfcShrink functions. The log₂ fold change ranked gene list was further used to run GSEA with the function ‘fgsea’ from the Bioconductor R package FGSEA (v1.8.0) (64) using the cancer hallmark pathways (h.all.v6.2.symbols.gmt) (65, 66) with default parameters. Pathways and signatures with adjusted p-value>0.05 were selected as significantly enriched pathways.

SUPPLEMENTARY TABLE 1
D. - Spatial barcode adapter sequences.

| Spatial | | |
|---|---|---|
| Barcode | | |
| oligos of | | |
| SNuBar | Sequences | SEQ ID |
| Name | | NO: |
| SNuBar_RN | CGAGCCCACGAGACCCTTGGCACCCGAGAATTCCAAGTATGC | |
| A-I7-1bc | TCCTTCCGTCCAAAAAAAAAAAAAAAAAAAAAAAAAAAAAA | |
| SNuBar_RN | CGAGCCCACGAGACCCTTGGCACCCGAGAATTCCAGCGACGC | |
| A-I7-2bc | AGATAAACCCTAAAAAAAAAAAAAAAAAAAAAAAAAAAAAA | |
| SNuBar_RN | CGAGCCCACGAGACCCTTGGCACCCGAGAATTCCACCATCTG | |
| A-I7-3bc | AGGTGTCAGCTAAAAAAAAAAAAAAAAAAAAAAAAAAAAAA | |
| SNuBar_RN | CGAGCCCACGAGACCCTTGGCACCCGAGAATTCCACGTTGTA | |
| A-I7-4bc | CTCAGATCTGTAAAAAAAAAAAAAAAAAAAAAAAAAAAAAA | |
| SNuBar_RN | CGAGCCCACGAGACCCTTGGCACCCGAGAATTCCAGACCTTG | |
| A-I7-5bc | CGTTATTAACTAAAAAAAAAAAAAAAAAAAAAAAAAAAAAA | |
| SNuBar_RN | CGAGCCCACGAGACCCTTGGCACCCGAGAATTCCAAGTTGTG | |
| A-I7-6bc | TAAGCGGCCCAAAAAAAAAAAAAAAAAAAAAAAAAAAAAA | |
| SNuBar_RN | CGAGCCCACGAGACCCTTGGCACCCGAGAATTCCAACTTGCG | |
| A-I7-7bc | TCCCTGCGAGTAAAAAAAAAAAAAAAAAAAAAAAAAAAAAA | |
| SNuBar_RN | CGAGCCCACGAGACCCTTGGCACCCGAGAATTCCAACGGAGA | |
| A-I7-8bc | GTACTAAATCTAAAAAAAAAAAAAAAAAAAAAAAAAAAAAA | |
| SNuBar_RN | CGAGCCCACGAGACCCTTGGCACCCGAGAATTCCAACATTGC | |
| A-I7-9bc | GACCCTTTATCAAAAAAAAAAAAAAAAAAAAAAAAAAAAAA | |
| SNuBar_RN | CGAGCCCACGAGACCCTTGGCACCCGAGAATTCCAGCCTTCG | |
| A-I7-10bc | ATGTACGATTTAAAAAAAAAAAAAAAAAAAAAAAAAAAAAA | |
| SNuBar_RN | CGAGCCCACGAGACCCTTGGCACCCGAGAATTCCAGCTCGAG | |
| A-I7-11bc | GCAACGTACCGAAAAAAAAAAAAAAAAAAAAAAAAAAAAAA | |
| SNuBar_RN | CGAGCCCACGAGACCCTTGGCACCCGAGAATTCCACCGTCAT | |
| A-I7-12bc | TGTGTTAGGACAAAAAAAAAAAAAAAAAAAAAAAAAAAAAA | |
| SNuBar_RN | CGAGCCCACGAGACCCTTGGCACCCGAGAATTCCACGTTTCG | |
| A-I7-13bc | GGCTCGAATCTAAAAAAAAAAAAAAAAAAAAAAAAAAAAAA | |
| SNuBar_RN | CGAGCCCACGAGACCCTTGGCACCCGAGAATTCCACACTAGA | |
| A-I7-14bc | ATAACGGCCGCAAAAAAAAAAAAAAAAAAAAAAAAAAAAAA | |
| SNuBar_RN | CGAGCCCACGAGACCCTTGGCACCCGAGAATTCCACTATGCT | |
| A-I7-15bc | CACGTTACGCCAAAAAAAAAAAAAAAAAAAAAAAAAAAAAA | |
| SNuBar_RN | CGAGCCCACGAGACCCTTGGCACCCGAGAATTCCATACATCT | |
| A-I7-16bc | CCCGGTGCCTGAAAAAAAAAAAAAAAAAAAAAAAAAAAAAA | |
| SNuBar_RN | CGAGCCCACGAGACCCTTGGCACCCGAGAATTCCAGATGAAC | |
| A-I7-I7bc | CTAGTCCCGTGAAAAAAAAAAAAAAAAAAAAAAAAAAAAAA | |
| SNuBar_RN | CGAGCCCACGAGACCCTTGGCACCCGAGAATTCCATATAAGG | |
| A-I7-18bc | TGGTCCGCCAGAAAAAAAAAAAAAAAAAAAAAAAAAAAAAA | |
| SNuBar_RN | CGAGCCCACGAGACCCTTGGCACCCGAGAATTCCACAGCTGA | |
| A-I7-19bc | GCTTATTATAGAAAAAAAAAAAAAAAAAAAAAAAAAAAAAA | |
| SNuBar_RN | CGAGCCCACGAGACCCTTGGCACCCGAGAATTCCATAACAAT | |
| A-I7-20bc | CCTCTTAAGTAAAAAAAAAAAAAAAAAAAAAAAAAAAAAA | |
| SNuBar_RN | CGAGCCCACGAGACCCTTGGCACCCGAGAATTCCAGACCGAG | |
| A-I7-21bc | GCGACGCCAATAAAAAAAAAAAAAAAAAAAAAAAAAAAAAA | |
| SNuBar_RN | CGAGCCCACGAGACCCTTGGCACCCGAGAATTCCACTAGGAC | |
| A-I7-22bc | GTCAAGATGACAAAAAAAAAAAAAAAAAAAAAAAAAAAAAA | |
| SNuBar_RN | CGAGCCCACGAGACCCTTGGCACCCGAGAATTCCATGTTCCG | |
| A-I7-23bc | ATGGGAGAAGCAAAAAAAAAAAAAAAAAAAAAAAAAAAAA | |
| | A | |
| SNuBar_RN | CGAGCCCACGAGACCCTTGGCACCCGAGAATTCCAGGTTAGC | |
| A-I7-24bc | CAAGGAGTATTAAAAAAAAAAAAAAAAAAAAAAAAAAAAAA | |
| SNuBar_RN | CGAGCCCACGAGACCCTTGGCACCCGAGAATTCCATTGCCGA | |
| A-I7-25bc | TGCGCGTAACCAAAAAAAAAAAAAAAAAAAAAAAAAAAAAA | |
| SNuBar_RN | CGAGCCCACGAGACCCTTGGCACCCGAGAATTCCACTCACGC | |
| A-I7-26bc | CTAGACCACTGAAAAAAAAAAAAAAAAAAAAAAAAAAAAAA | |
| SNuBar_RN | CGAGCCCACGAGACCCTTGGCACCCGAGAATTCCATTTAGCTC | |
| A-I7-27bc | CGCTCAACGCAAAAAAAAAAAAAAAAAAAAAAAAAAAAAA | |
| SNuBar_RN | CGAGCCCACGAGACCCTTGGCACCCGAGAATTCCACTACATG | |
| A-I7-28bc | TCGACGTTGGTAAAAAAAAAAAAAAAAAAAAAAAAAAAAAA | |
| SNuBar_RN | CGAGCCCACGAGACCCTTGGCACCCGAGAATTCCACGGAGGT | |
| A-I7-29bc | ATGCTATATTTAAAAAAAAAAAAAAAAAAAAAAAAAAAAAA | |
| SNuBar_RN | CGAGCCCACGAGACCCTTGGCACCCGAGAATTCCACGTTCTAT | |
| A-I7-30bc | AACCACTCGTAAAAAAAAAAAAAAAAAAAAAAAAAAAAAA | |
| SNuBar_RN | CGAGCCCACGAGACCCTTGGCACCCGAGAATTCCACACGATT | |
| A-I7-31bc | AGGGTTCGTCGAAAAAAAAAAAAAAAAAAAAAAAAAAAAAA | |
| SNuBar_RN | CGAGCCCACGAGACCCTTGGCACCCGAGAATTCCATGGAGAA | |
| A-I7-32bc | CTCTCGGTAGCAAAAAAAAAAAAAAAAAAAAAAAAAAAAAA | |
| SNuBar_RN | CGAGCCCACGAGACCCTTGGCACCCGAGAATTCCATTCAACC | |
| A-I7-33bc | ACTGTGACAGTAAAAAAAAAAAAAAAAAAAAAAAAAAAAAA | |
| SNuBar_RN | CGAGCCCACGAGACCCTTGGCACCCGAGAATTCCATACCAGT | |
| A-I7-34bc | TCTAGATGTTAAAAAAAAAAAAAAAAAAAAAAAAAAAAAA | |
| SNuBar_RN | CGAGCCCACGAGACCCTTGGCACCCGAGAATTCCATCATACG | |
| A-I7-35bc | GGCGTAATGCCAAAAAAAAAAAAAAAAAAAAAAAAAAAAAA | |
| SNuBar_RN | CGAGCCCACGAGACCCTTGGCACCCGAGAATTCCACTTCCAA | |
| A-I7-36bc | CTCTCGCATAGAAAAAAAAAAAAAAAAAAAAAAAAAAAAAA | |

Barcode adapters 1, and 16-18 were used in the four cell line mixture experiments, while barcode adapters 1-36 were used in the normal breast tissue experiment, and barcode adapters 1-15 were used in the frozen breast tumor experiment.

### E. References for Example 4

The following references and the publications referred to throughout the specification, to the extent that they provide exemplary procedural or other details supplementary to those set forth herein, are specifically incorporated herein by reference.
1. Wang, M. et al. Role of tumor microenvironment in tumorigenesis. J Cancer 8, 761-773 (2017).
2. Javed, A. & Lteif, A. Development of the Human Breast. Seminars in Plastic Surgery 27, 005-012 (2013).
3. Macias, H. & Hinck, L. Mammary gland development. Wiley Interdisciplinary Reviews: Developmental Biology 1, 533-557 (2012).
4. Nguyen, Q. H. et al. Profiling human breast epithelial cells using single cell RNA sequencing identifies cell diversity. Nature Communications 9, 2028 (2018).
5. Chung, W. et al. Single-cell RNA-seq enables comprehensive tumour and immune cell profiling in primary breast cancer. Nature Communications 8, 15081 (2017).
6. Yin, J. et al. Comprehensive analysis of immune evasion in breast cancer by single-cell RNA-seq. bioRxiv 368605 (2018). doi:10.1101/368605
7. Murrow, L. M. et al. Mapping the complex paracrine response to hormones in the human breast at single-cell resolution. bioRxiv 430611 (2018). doi:10.1101/430611
8. Kobayashi, H. et al. Cancer-associated fibroblasts in gastrointestinal cancer. Nature Reviews Gastroenterology & Hepatology 1 (2019). doi:10.1038/s41575-019-0115-0
9. Hendry, S. et al. Assessing tumor infiltrating lymphocytes in solid tumors: a practical review for pathologists and proposal for a standardized method from the International Immuno-Oncology Biomarkers Working Group. Adv Anat Pathol 24, 235-251 (2017).
10. Noy, R. & Pollard, J. W. Tumor-associated macrophages: from mechanisms to therapy. Immunity 41, 49-61 (2014).
11. Dudley, A. C. Tumor Endothelial Cells. Cold Spring Harb Perspect Med 2, (2012).
12. Gierahn, T. M. et al. Seq-Well: portable, low-cost RNA sequencing of single cells at high throughput. Nat. Methods 14, 395-398 (2017).
13. Macosko, E. Z. et al. Highly Parallel Genome-wide Expression Profiling of Individual Cells Using Nanoliter Droplets. Cell 161, 1202-1214 (2015).
14. Han, X. et al. Mapping the Mouse Cell Atlas by Microwell-Seq. Cell 172, 1091-1107.e17 (2018).
15. Klein, A. M. et al. Droplet Barcoding for Single-Cell Transcriptomics Applied to Embryonic Stem Cells. Cell 161, 1187-1201 (2015).
16. Gao, R. et al. Nanogrid single-nucleus RNA sequencing reveals phenotypic diversity in breast cancer. Nature Communications 8, 228 (2017).
17. Habib, N. et al. Massively parallel single-nucleus RNA-seq with DroNc-seq. Nat. Methods 14, 955-958 (2017).
18. Stahl, P. L. et al. Visualization and analysis of gene expression in tissue sections by spatial transcriptomics. Science 353, 78-82 (2016).
19. Vickovic, S. et al. High-density spatial transcriptomics arrays for in situ tissue profiling. bioRxiv 563338 (2019). doi:10.1101/563338
20. Rodrigues, S. G. et al. Slide-seq: A scalable technology for measuring genome-wide expression at high spatial resolution. Science 363, 1463-1467 (2019).
21. Lee, J. H. et al. Fluorescent in situ sequencing (FISSEQ) of RNA for gene expression profiling in intact cells and tissues. Nature Protocols 10, 442-458 (2015).
22. Raj, A., van den Bogaard, P., Rifkin, S. A., van Oudenaarden, A. & Tyagi, S. Imaging individual mRNA molecules using multiple singly labeled probes. Nature Methods 5, 877-879 (2008).
23. Shah, S., Lubeck, E., Zhou, W. & Cal, L. seqFISH Accurately Detects Transcripts in
Single Cells and Reveals Robust Spatial Organization in the Hippocampus. Neuron 94, 752-758.el (2017).
24. Moffitt, J. R. et al. Molecular, spatial, and functional single-cell profiling of the hypothalamic preoptic region. Science 362, eaau5324 (2018).
25. Eng, C.-H. L. et al. Transcriptome-scale super-resolved imaging in tissues by RNA seqFISH+. Nature 568, 235 (2019).
26. Haghverdi, L., Lun, A. T. L., Morgan, M. D. & Marioni, J. C. Batch effects in single-cell RNA-sequencing data are corrected by matching mutual nearest neighbors. Nature Biotechnology 36, 421-427 (2018).
27. Stegle, O., Teichmann, S. A. & Marioni, J. C. Computational and analytical challenges in single-cell transcriptomics. Nature Reviews Genetics 16, 133-145 (2015).
28. Lun, A. T. L., McCarthy, D. J. & Marioni, J. C. A step-by-step workflow for low-level analysis of single-cell RNA-seq data with Bioconductor. F1000Res 5, 2122 (2016).
29. Wolock, S. L., Lopez, R. & Klein, A. M. Scrublet: Computational Identification of Cell Doublets in Single-Cell Transcriptomic Data. Cell Systems 8, 281-291.e9 (2019).
30. Moritani, S. et al. Immunohistochemical expression of myoepithelial markers in adenomyoepithelioma of the breast: a unique paradoxical staining pattern of high-molecular weight cytokeratins. Virchows Arch. 466, 191-198 (2015).
31. Stingl, J., Eaves, C. J., Zandieh, I. & Emerman, J. T. Characterization of bipotent mammary epithelial progenitor cells in normal adult human breast tissue. Breast Cancer Res. Treat. 67, 93-109 (2001).
32. Uhlén, M. et al. Proteomics. Tissue-based map of the human proteome. Science 347, 1260419 (2015).
33. Tirosh, I. et al. Dissecting the multicellular ecosystem of metastatic melanoma by single-cell RNA-seq. Science 352, 189-196 (2016).
34. Inoue, H., Ichinose, M., Miura, M., Katsumata, U. & Takishima, T. Sensory receptors and reflex pathways of nonadrenergic inhibitory nervous system in feline airways. Am. Rev. Respir. Dis. 139, 1175-1178 (1989).
35. Ceredig, R. & Rolink, T. A positive look at double-negative thymocytes. Nat. Rev. Immunol. 2, 888-897 (2002).
36. Chung, S., Sawyer, J. K., Gebre, A. K., Maeda, N. & Parks, J. S. Adipose tissue ATP binding cassette transporter A1 contributes to high-density lipoprotein biogenesis in vivo. Circulation 124, 1663-1672 (2011).
37. Schmitz, G. & Langmann, T. Structure, function and regulation of the ABC1 gene product. Curr. Opin. Lipidol. 12, 129-140 (2001).
38. Phillips, M. C. Molecular mechanisms of cellular cholesterol efflux. J. Biol. Chem. 289, 24020-24029 (2014).
39. Rundhaug, J. E. Matrix metalloproteinases and angiogenesis. J. Cell. Mol. Med. 9, 267-285 (2005).
40. Krock, B. L., Skuli, N. & Simon, M. C. Hypoxia-induced angiogenesis: good and evil. Genes Cancer 2, 1117-1133 (2011).
41. Fantin, A. et al. NRP1 acts cell autonomously in endothelium to promote tip cell function during sprouting angiogenesis. Blood 121, 2352-2362 (2013).
42. Coffelt, S. B. et al. Angiopoietin-2 regulates gene expression in TIE2-expressing monocytes and augments their inherent proangiogenic functions. Cancer Res. 70, 5270-5280 (2010).
43. Naldini, A. et al. Cutting edge: IL-1beta mediates the proangiogenic activity of osteopontin-activated human monocytes. J. Immunol. 177, 4267-4270 (2006).
44. Medina, R. J. et al. Myeloid angiogenic cells act as alternative M2 macrophages and modulate angiogenesis through interleukin-8. Mol. Med. 17, 1045-1055 (2011).
45. Kzhyshkowska, J. et al. Role of tumor associated macrophages in tumor angiogenesis and lymphangiogenesis. Front. Physiol. 5, (2014).
46. Murdoch, C., Muthana, M., Coffelt, S. B. & Lewis, C. E. The role of myeloid cells in the promotion of tumour angiogenesis. Nat. Rev. Cancer 8, 618-631 (2008).
47. Elliott, L. A., Doherty, G. A., Sheahan, K. & Ryan, E. J. Human Tumor-Infiltrating Myeloid Cells: Phenotypic and Functional Diversity. Front Immunol 8, 86 (2017).
48. Collin, M. & Bigley, V. Human dendritic cell subsets: an update. Immunology 154, 3-20 (2018).
49. Gudjonsson, T., Adriance, M. C., Sternlicht, M. D., Petersen, 0. W. & Bissell, M. J. Myoepithelial cells: their origin and function in breast morphogenesis and neoplasia. J Mammary Gland Biol Neoplasia 10, 261-272 (2005).
50. Betterman, K. L. et al. Remodeling of the lymphatic vasculature during mouse mammary gland morphogenesis is mediated via epithelial-derived lymphangiogenic stimuli. Am. J. Pathol. 181, 2225-2238 (2012).
51. Costa, A. et al. Fibroblast Heterogeneity and Immunosuppressive Environment in Human Breast Cancer. Cancer Cell 33, 463-479.e10 (2018).
52. Kaur, H. et al. Next-generation sequencing: a powerful tool for the discovery of molecular markers in breast ductal carcinoma in situ. Expert Rev. Mol. Diagn. 13, 151-165 (2013).
53. Bastien, R. R. L. et al. PAM50 breast cancer subtyping by RT-qPCR and concordance with standard clinical molecular markers. BMC Med Genomics 5, 44 (2012).
54. Liberzon, A. et al. The Molecular Signatures Database Hallmark Gene Set Collection. cells 1, 417-425 (2015).
55. Stoeckius, M. et al. Cell ‘hashing’ with barcoded antibodies enables multiplexing and doublet detection for single cell genomics. bioRxiv (2017). doi:10.1101/237693
56. McGinnis, C. S. et al. MULTI-seq: sample multiplexing for single-cell RNA sequencing using lipid-tagged indices. Nature Methods 16, 619 (2019).
57. Wolfe, J. & Bryant, G. Freezing, drying, and/or vitrification of membrane-solute-water systems. Cryobiology 39, 103-129 (1999).
58. Wu, H., Kirita, Y., Donnelly, E. L. & Humphreys, B. D. Advantages of Single-Nucleus over Single-Cell RNA Sequencing of Adult Kidney: Rare Cell Types and Novel Cell States Revealed in Fibrosis. J. Am. Soc. Nephrol. 30, 23-32 (2019).
59. Lake, B. B. et al. Neuronal subtypes and diversity revealed by single-nucleus RNA sequencing of the human brain. Science 352, 1586-1590 (2016).
60. Leung, M. L. et al. Highly multiplexed targeted DNA sequencing from single nuclei. Nature Protocols 11, 214-235 (2016).
61. Butler, A., Hoffman, P., Smibert, P., Papalexi, E. & Satija, R. Integrating single-cell transcriptomic data across different conditions, technologies, and species. Nature Biotechnology 36, 411-420 (2018).
62. Patrick Roelli, bbimber, Bill Flynn, santiagorevale & Gege Gui. Hoohm/CITE-seq-Count. 1.4.2. (Zenodo, 2019). doi:10.5281/zenodo.2590196
63. Love, M. I., Huber, W. & Anders, S. Moderated estimation of fold change and dispersion for RNA-seq data with DESeq2. Genome Biology 15, 550 (2014).
64. Sergushichev, A. A. An algorithm for fast preranked gene set enrichment analysis using cumulative statistic calculation. bioRxiv 060012 (2016). doi:10.1101/060012
65. Subramanian, A. et al. Gene set enrichment analysis: A knowledge-based approach for interpreting genome-wide expression profiles. PNAS 102, 15545-15550 (2005).
66. Mootha, V. K. et al. PGC-1α-responsive genes involved in oxidative phosphorylation are coordinately downregulated in human diabetes. Nature Genetics 34, 267-273 (2003).

### Example 5: In Situ Spatial Barcoding in Tissue

### A. Gasket-Based SNuBar

To show that SNUBAR can also applied to barcode single nuclei in tissue sections, the inventors tested the transposome barcoding system on 4 different tissues types (Mouse lung, Mouse tissue, Human breast cancer sample, and normal human breast tissue) using 3.5 mm×3.5 mm/well gaskets to separate different spatial tissue regions of the same section. Tissues were first cryo-sectioned into 25 μm thickness sections and mounted on top of glass slides, then lysed with lysis buffer and wash twice with PBS/BSA buffer. The gasket was assembled on top of the slides. Then the inventors added 14 ul of wash buffer, 15 ul 2×TD buffer and 1 ul barcoded transposome and incubated for 20 min at 37° C. The transposome was inactivated with the NST buffer, and the tissues were scrapped from the slides and collected as barcoded nuclear suspensions, then passed through 40 um filters, and centrifuged at 800 g for 5 min at 4° C. Filtered nuclei were used to prepare high throughput single cell RNA sequencing libraries on the 10× Genomics 3′ RNA platform.

### B. Microarray-based SNuBar

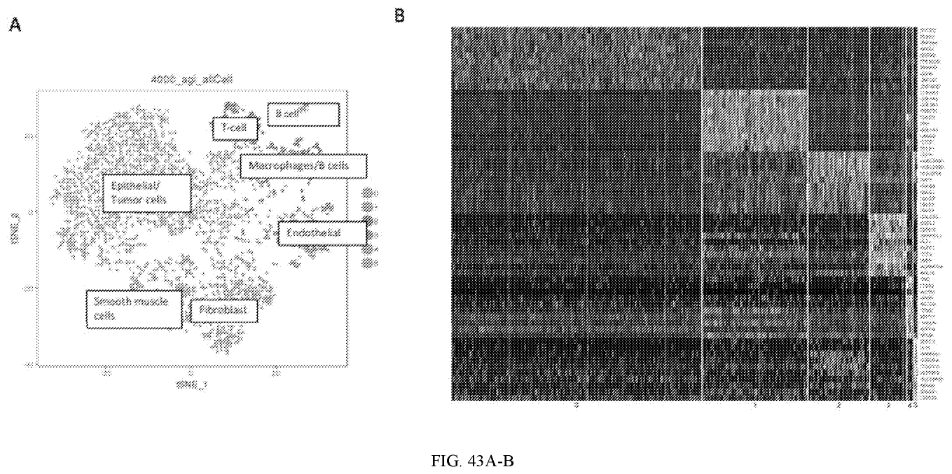

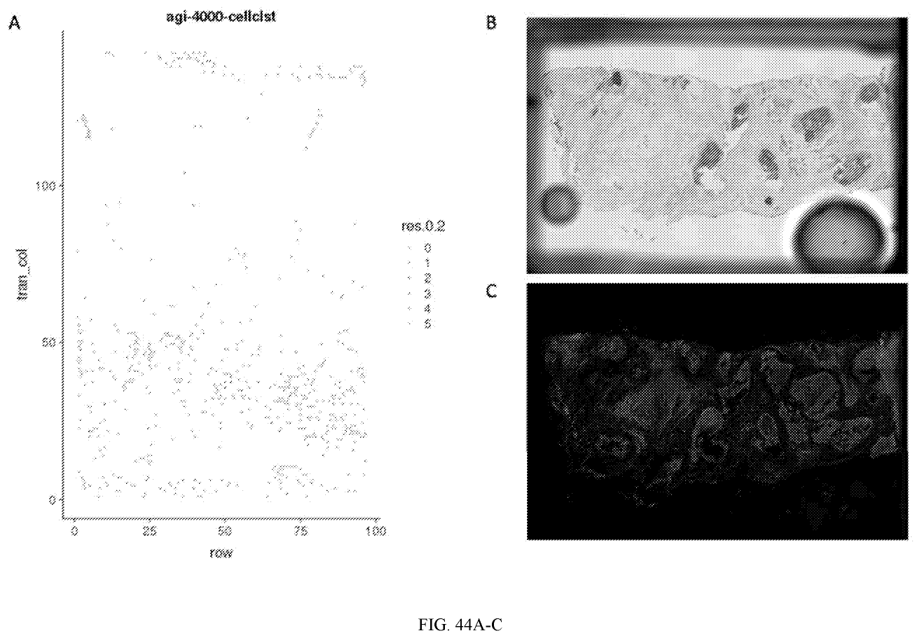

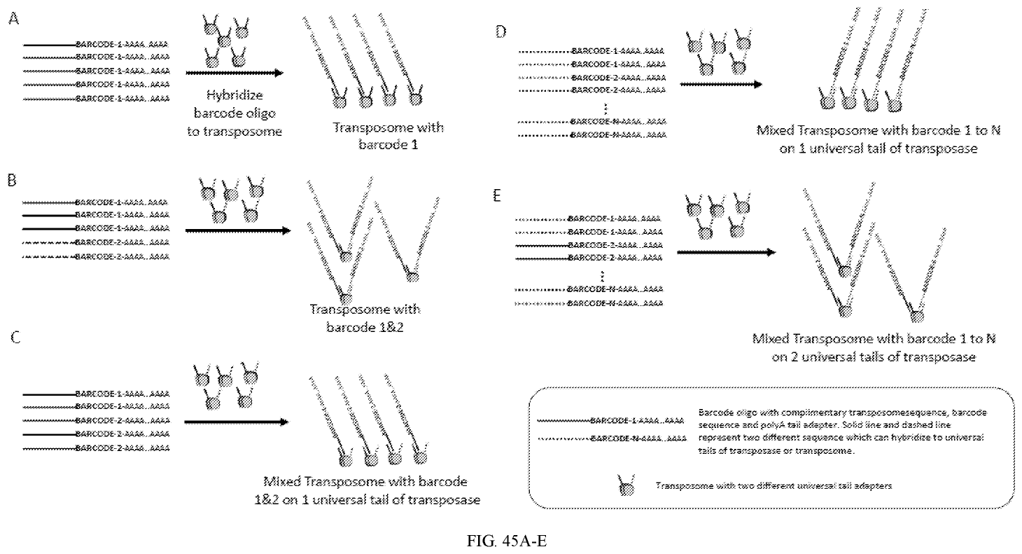

To person barcoding of single nuclei in situ with high spatial resolution, the inventors designed a customiz 8×15 k high density DNA microarray (Agilent) with spatial barcodes printed in the spots, where the diameter of each feature is 65 um and could cover about 5-20 single cells, the microarray was then hybridized with a bridge oligo and transposome. Human tissue sample from patients with ductal carcinoma in situ (DCIS) were cut into 20 um thickness and mounted on glass slides, then lysed with 100 ul (DAPI/NST+0.2 U/ul RNase Inhibitor) buffer for 15 min on ice. Lysis buffer was removed and washed with wash buffer (PBS, 0.04% BSA, 0.2 U/ul RNase Inhibitor, DAPI) three times and imaged on the EVOSII (DAPI stain and bright field). The inventors then removed the wash buffer and added 10 ul the master mix to each array (T4 DNA ligase buffer: 1 ul, BamHI (100 U/ul): 1.5 ul, RNase Inhibitor, Murine (40 U/ul), Final (1 U/ul): 0.25 ul, H20: 7.5 ul). Then, covered the assembled barcoded DNA microarray and seal the slides, followed by incubation at 37° C. for 30 min. Next we scrapped the tissue into tubes and passed the it through 40 um filters, followed by QC analysis of the cells using EVOS and Countness II, followed by centrifugation at 500 g for 5 min at 4° C. The inventors then pipetted the supernatant out (left 50 ul) and washed it with 900 ul PBS+BSA(1%)+0.2 U/ul RNase Inhibitor buffer twice, and resuspend the cells with ˜10-20 ul PBS/1% BSA buffer. Next, we counted the cells with Countness II (˜5×10⁵/ml), and picked up 15 ul to perform 3′ RNA-seq (10× Genomics) and sequenced 1 lane on the Nextseq500 system (Illumina Inc.). In total, the inventors sequenced ˜4000 single cells with 88,078 reads per cell, and 1,296 gene per cell. We identified 6 different major cell types including epithelial cells, fibroblast, immune cells (T cell, macrophage, B cell), endothelial and smooth muscle cells (FIG. 43A-B). Because we could resolve the spatial barcodes for each single cell, we were able to map all of the single cells to their X-Y tissue coordinates according to their spatial barcodes (FIG. 44A). The majority of the cells mapped to the bottom part of the microarray, which corresponds to the region where we placed the tissue section on the microarray (FIG. 44B-C). and shows that regions with ducts have more cells, as was expected. These data suggest that the custom microarray delivery method can efficiently barcode single cells in situ using the SNUBAR approach.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references and the publications referred to throughout the specification, to the extent that they provide exemplary procedural or other details supplementary to those set forth herein, are specifically incorporated herein by reference.
1. Hwang, B., J. H. Lee, and D. Bang, Single-cell RNA sequencing technologies and bioinformatics pipelines. Experimental & Molecular Medicine, 2018. 50(8): p. 96.
2. Macosko, Evan Z., et al., Highly Parallel Genome-wide Expression Profiling of Individual Cells Using Nanoliter Droplets. Cell, 2015. 161(5): p. 1202-1214.
3. Klein, Allon M., et al., Droplet Barcoding for Single-Cell Transcriptomics Applied to Embryonic Stem Cells. Cell, 2015. 161(5): p. 1187-1201.
4. Gierahn, T. M., et al., Seq-Well: portable, low-cost RNA sequencing of single cells at high throughput. Nature Methods, 2017. 14: p. 395.
5. Han, X., et al., Mapping the Mouse Cell Atlas by Microwell-Seq. Cell, 2018. 172(5): p. 1091-1107.e17.
6. Gao, R., et al., Nanogrid single-nucleus RNA sequencing reveals phenotypic diversity in breast cancer. Nature Communications, 2017. 8(1): p. 228.
7. Zheng, G. X. Y., et al., Massively parallel digital transcriptional profiling of single cells. Nature Communications, 2017. 8: p. 14049.
8. Ramskold, D., et al., Full-length mRNA-Seq from single-cell levels of RNA and individual circulating tumor cells. Nature Biotechnology, 2012. 30: p. 777.
9. Picelli, S., et al., Full-length RNA-seq from single cells using Smart-seq2. Nature Protocols, 2014. 9: p. 171.
10. Hashimshony, T., et al., CEL-Seq: Single-Cell RNA-Seq by Multiplexed Linear Amplification. Cell Reports, 2012. 2(3): p. 666-673.
11. Hashimshony, T., et al., CEL-Seq2: sensitive highly-multiplexed single-cell RNA-Seq.

Genome Biology, 2016. 17(1): p. 77.
12. Vitak, S. A., et al., Sequencing thousands of single-cell genomes with combinatorial indexing. Nature Methods, 2017. 14: p. 302.
13. Zahn, H., et al., Scalable whole-genome single-cell library preparation without preamplification. Nature Methods, 2017. 14: p. 167.
14. Cusanovich, D. A., et al., Multiplex single-cell profiling of chromatin accessibility by combinatorial cellular indexing. Science, 2015. 348(6237): p. 910.
15. Mezger, A., et al., High-throughput chromatin accessibility profiling at single-cell resolution. bioRxiv, 2018.

1. A method for barcoding eukaryotic cell nuclei comprising: transferring a plurality of oligonucleotides into the nuclei of a plurality of cells and performing single-cell analysis to identify the sequence of the barcode; wherein each oligonucleotide comprises a barcode region and a target region.

2. The method of claim 1, wherein the oligonucleotide is transferred into the nuclei of cells in a transposome complex.

3. The method of claim 2, wherein the oligonucleotide further comprises a transposome adaptor region.

4. The method of any one of claims 1-3, wherein the barcode corresponds to a cellular characteristic, wherein the characteristic comprises a location of the cell in a tissue, a cell type, a clonal population of cells, a patient sample, or a treatment condition.

5. The method of claim 4, wherein the clonal population of cells comprises a clonal population of cancerous cells.

6. The method of claim 4, wherein the cells are within a tissue, and the cellular characteristic comprises the location of the cell within a tissue.

7. The method of claim 6, wherein at least two cells at different locations in a tissue are each barcoded with a different barcode corresponding to the respective tissue locations of each of the cells.

8. The method of claim 4, wherein the cellular characteristic is a cell type, and wherein a first barcode corresponds to cells from a first cell type and a second barcode corresponds to cells from a second cell type.

9. The method of claim 4, wherein the cellular characteristic is a patient sample, and wherein a first barcode corresponds to cells from a first patient sample and a second barcode corresponds to cells from a second patient sample.

10. The method of claim 4, wherein the cellular characteristic is the location of the cell within a tissue, and wherein a first barcode corresponds to a first location and a second barcode corresponds to a second location.

11. The method of claim 10, wherein the total area of barcoded cells within the tissue is greater than 1 mm².

12. The method of claim 4, wherein the cellular characteristic is a treatment condition, and wherein a first barcode corresponds to a first treatment condition and a second barcode corresponds to a second treatment condition.

13. The method of any one of claims 1-12, wherein the method further comprises combining the barcoded nuclei in a suspension and wherein the nuclear envelope of the barcoded nuclei is intact in the suspension.

14. The method of any one of claims 1-13, wherein the method further comprises performing single-cell analysis of nucleic acids from the cellular nuclei.

15. The method of claim 14, wherein the single-cell analysis comprises sequencing nucleic acids to determine the sequence of the barcode(s).

16. The method of claim 14 or 15, wherein the single-cell analysis comprises sequencing cellular nucleic acids to determine the transcription or genomic profile of the single cell.

17. The method of claim 16, wherein the transcription or genomic profile comprises the profile of at least 1000 genes of the single cell.

18. The method of any one of claims 15-17, wherein at least 2000 different barcodes are sequenced.

19. The method of any one of claims 1-18, wherein each cell contains exactly one or two exogenously added barcodes.

20. The method of claim 19, wherein each cell contains two exogenously added barcodes and wherein the combination of the sequence of the two barcodes correspond to a cellular characteristic of each cell.

21. The method of any one of claims 2-19, wherein each transposome complex comprises one or two oligonucleotides.

22. The method of claim 21, wherein the transposome complex comprises at least two oligonucleotides.

23. The method of claim 22, wherein the transposome complex comprises at least a first oligonucleotide comprising a first barcode and a second oligonucleotide comprising a second barcode and wherein the first and second barcode are different.

24. The method of any one of claims 14-20, wherein the single-cell analysis comprises determining the proteomic profile of the single cell.

25. The method of any one of claims 14-24, wherein the single-cell analysis comprises sequencing the nucleic acids.

26. The method of any one of claims 14-25, wherein the nucleic acids comprise RNA.

27. The method of any one of claims 14-26, wherein the single-analysis involves single-cell RNA sequencing to determine, quantitate, or identify one or more of RNA splicing, RNA-protein interaction, RNA modification, RNA structure or lincRNA, microRNA, mRNA, tRNA and circRNA analysis.

28. The method of claim 26 or 27, wherein the analysis comprises one or more of drop-seq, InDrop, seq-well, fluidigm, BD biosciences, illumina bio-rad microdroplets, sci-seq microwell-seq, nanogrid-seq, 10× genomics RNA sequencing platform, SMART-seq, SMART-seq2, CEL-seq, CEL-seq2.

29. The method of claim 14 or 25, wherein the nucleic acids comprise DNA.

30. The methods of claim 29, wherein the single-cell analysis comprises one or more of single cell DNA copy number profiling, single cell mutation detection, single cell structural variant detection, detection of DNA and protein interactions, DNA chromatin profiling, detection of DNA-DNA interactions, and detection of DNA epigenetic modifications.

31. The method of claim 29, wherein the single-cell analysis comprises one or more of 10× genomics CNV sequencing platform, mission bio, fluidigm, sci-seq, direct-tagmentation, sciATAC-seq, nano-well scATAC-seq, MDA, DOP-PCR, MALBAC, and LIANTI.

32. The method of any one of claims 1-31, wherein the nuclei is derived from or within a eukaryotic cell that is greater than 50 microns.

33. The method of any one of claims 1-32, wherein the nuclei is derived from or within a eukaryotic cell that comprises an irregular morphology.

34. The method of any one of claims 1-33, wherein the nuclei is derived from or within a eukaryotic cell that has been previously frozen.

35. The method of any one of claims 1-34, wherein the barcode sequence is non-contiguous with endogenous DNA or RNA sequences.

36. The method of any one of claims 14-35, wherein the method further comprises isolating nucleic acids from the cells.

37. The method of any one of claims 2-36, wherein the transposome adaptor region comprises a transposase recognition sequence.

38. The method of any one of claims 2-37, wherein the transposome adaptor region comprises a complementary sequence capable of base-pairing with a transposome nucleic acid component.

39. The method of any one of claims 1-38, wherein the plurality of oligos comprises at least one oligo comprising a transposase recognition sequence and at least one oligo comprising a complementary sequence capable of base-pairing with a transposome nucleic acid component.

40. The method of any one of claims 1-39, wherein the method further comprises fragmentation of nucleic acids endogenous to the cell.

41. The method of claim 40, wherein the fragmentation is performed prior to transferring the plurality of oligonucleotides into the plurality of cells.

42. The method of any one of claims 1-41, wherein the target region comprises one or more primer binding sites.

43. The method of any one of claims 1-42, wherein the target region comprises a poly adenine region comprising at least 4 consecutive adenine nucleic acids.

44. The method of any one of claims 1-43, wherein the target region comprises a universal primer binding region and a random primer binding region.

45. The method of any one of claims 1-44, wherein transferring the oligonucleotides into the cell comprises micropipetting oligonucleotides into or on top of each nucleus; printing oligonucleotides into or on top of each nucleus; releasing oligonucleotides from a substrate with cells deposited on top of the oligonucleotides and substrate; and acoustic liquid transfer of oligonucleotides to each nucleus.

46. The method of claim 45, wherein the oligonucleotide further comprises a cleavage site.

47. The method of claim 45 or 46, wherein releasing oligonucleotides comprises restriction enzyme cleavage, nickase cleavage, UV photocleavage, or chemical cleavage of the oligonucleotide.

48. The method of any one of claims 45-47, wherein the substrate comprises a microarray.

49. The method of any one of claims 1-45, wherein the oligonucleotides are transferred to cell nuclei, and wherein the cells are in an endogenous location within a tissue section.

50. The method of any one of claims 25-49, wherein the sequence comprising the barcode does not comprise sequences from the cellular nucleic acids.

51. The method of any one of claims 1-50, wherein the transposome comprises Tn5, Sleeping Beauty, PiggyBac, Tn7 or MuA.

52. A method for barcoding eukaryotic cell nuclei comprising:
i) transferring oligonucleotides into the nuclei of cells; wherein the oligonucleotides comprise a barcode region and a target region;
ii) combining the barcoded nuclei in a suspension and wherein the nuclear envelope of the barcoded nuclei is intact in the suspension; and
iii) performing single-cell analysis of the suspension to identify the sequence of the barcode and the transcriptomic, proteomic, and/or genomic profile of the cell;
wherein the barcode sequence is non-contiguous with endogenous DNA or RNA sequences and wherein the barcode corresponds to the endogenous location of a cell within a tissue section.
